(19) United States
(12) Patent Application Publication
(10) Pub. No.: US 2007/0167499 A1
(43) Pub. Date: Jul. 19, 2007

(54) Biaryl linked hydroxamates: preparation and pharmaceutical applications

(75) Inventors: Walter Stunkel, Singapore (SG); Haishan Wang, Singapore (SG); Zheng Yin, Singapore (SG)

Correspondence Address: CONNOLLY BOVE LODGE & HUTZ LLP, P.O. BOX 2207, WILMINGTON, DE, 19899-2207, US

(73) Assignee: A*BIO Pte Ltd., Singapore (SG)

(21) Appl. No.: 10/577,219

(22) PCT Filed: Oct. 26, 2004
(86) PCT No.: PCT/SG04/00354

§ 371 Date: Feb. 21, 2007

Related U.S. Application Data
(60) Provisional application No. 60/514,012, filed on Oct. 27, 2003.
(60) Provisional application No. 60/532,615, filed on Dec. 29, 2003.

(51) Int. Cl. A61K 31/433; A61K 31/4245; A61K 31/4196; C07D 417/02; C07D 413/02; C07D 403/02

(52) U.S. Cl. 514/362; 514/416; 514/364; 514/383; 548/138; 548/148; 548/266.2

(57) ABSTRACT

The present invention relates to hydroxamate compounds which are inhibitors of histone deacetylase. More particularly, the present invention relates to biaryl containing compounds and methods for their preparation. These compounds may be useful as medicaments for the treatment of proliferative disorders as well as other diseases involving, relating to or associated with enzymes having histone deacetylase activities. Formula (I), where Z is a single bond or C₁-C₄ hydrocarbon, A is an aromatic ring, B is an aromatic ring.

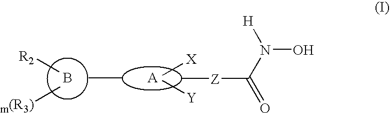

### FIELD OF THE INVENTION

The present invention relates to hydroxamate compounds that are inhibitors of histone deacetylase. More particularly, the present invention relates to biaryl containing compounds and methods for their preparation. These compounds may be useful as medicaments for the treatment of proliferative disorders as well as other diseases involving, relating to or associated with enzymes having histone deacetylase activities.

### BACKGROUND OF THE INVENTION

Local chromatin architecture is generally recognized as an important factor in the regulation of gene expression. The architecture of chromatin, a protein-DNA complex, is strongly influenced by post-translational modifications of the histones which are the protein components. Reversible acetylation of histones is a key component in the regulation of gene expression by altering the accessibility of transcription factors to DNA. In general, increased levels of histone acetylation are associated with increased transcriptional activity, whereas decreased levels of acetylation are associated with repression of gene expression [Wade P. A. Hum. Mol. Genet. 10, 693-698 (2001), De Ruijter A.J.M. et al, Biochem. J., 370, 737-749 (2003)]. In normal cells, histone deacetylases (HDACs) and histone acetyltransferase together control the level of acetylation of histones to maintain a balance. Inhibition of HDACs results in the accumulation of acetylated histones, which results in a variety of cell type dependent cellular responses, such as apoptosis, necrosis, differentiation, cell survival, inhibition of proliferation and cytostasis.

Inhibitors of HDAC have been studied for their therapeutic effects on cancer cells. For example, suberoylanilide hydroxamic acid (SAHA) is a potent inducer of differentiation and/or apoptosis in murine erythroleukemia, bladder, and myeloma cell lines [Richon V. M. et al, Proc. Natl. Acad. Sci. USA, 93: 5705-5708 (1996), Richon V. M. et al, Proc. Natl. Acad. Sci. USA, 95: 3003-3007 (1998)]. SAHA has been shown to suppress the growth of prostate cancer cells in vitro and In vivo [Butler L. M. et al, Cancer Res. 60, 5165-5170 (2000)]. Other inhibitors of HDAC that have been widely studied for their anti-cancer activities are trichostatin A (TSA) and trapoxin B [Yoshida M. et al, J. Biol. Chem., 265, 17174 (1990), Kijima M. et al, J. Biol. Chem., 268, 22429 (1993)]. Trichostatin A is a reversible inhibitor of mammalian HDAC. Trapoxin B is a cyclic tetrapeptide, which is an irreversible inhibitor of mammalian HDAC. However, due to the in vivo instability of these compounds they are less desirable as anti-cancer drugs. Recently, other small molecule HDAC inhibitors have become available for clinical evaluation [U.S. Pat. No. 6,552,065]. Additional HDAC inhibiting compounds have been reported in the literature [Bouchain G. et al, J. Med. Chem., 46, 820-830 (2003)] and patents [WO 03/066579A2, WO 01/38322 A1]. The in vivo activity of such inhibitors can be directly monitored by their ability to increase the amount of acetylated histones in the biological sample. HDAC inhibitors have been reported to interfere with neurodegenerative processes, for instance, HDAC inhibitors arrest polyglutamine-dependent neurodegeneration [Nature, 413(6857): 73943, 18 Oct. 2001]. In addition, HDAC inhibitors have also been known to inhibit production of cytokines such as TNF, IFN, IL-1 which are known to be implicated in inflammatory diseases and/or immune system disorders. [J. Biol. Chem. 1990; 265(18): 10230-10237; Science, 1998; 281: 1001-1005; Dinarello C. A. and Moldawer L. L. Proinflammatory and anti-inflammatory cytokines in rheumatoid arthritis. A primer for clinicians. 2^{nd} Edition, Amergen Inc., 2000].

Nevertheless, there is still a need to provide further HDAC inhibitors that would be expected to have useful, improved pharmaceutical properties in the treatment of diseases such as cancer, neurodegenerative diseases and inflammatory and/or immune system disorders.

### SUMMARY OF THE INVENTION

In one aspect the present invention provides compounds of the Formula (I): 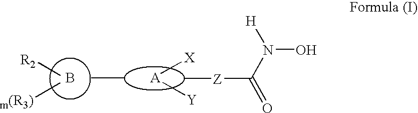
wherein
Z is a single bond or a C₁-C₄ hydrocarbon chain containing no more than 1 double or triple bond, optionally substituted with one or more substituents independently selected from the group consisting of C₁-C₄ alkyl;
A is an aromatic ring selected from the group consisting of optionally substtuted arylene and optionally substituted heteroarylene, wherein A is not benzimidazole and when Z is a single bond then A is not selected from the group consisting of phenylene and six-membered heteroarylene containing 3 or less than 3 nitrogens;
B is an aromatic ring selected from the group consisting of optionally substituted aryl, optionally substituted arylene, optionally substituted heteroaryl and optionally substituted heteroarylene and wherein A and B can not both be phenylene and wherein when Z is a single bond then B is not a bicyclic aryl or bicyclic heteroaryl;
wherein A and B are connected via a carbon-carbon bond;
R₂ is selected from the group consisting of halogen, alkyl, alkenyl, alkynyl, haloalkyl, haloalkenyl, heteroalkyl, cycloalkyl, cycloalkenyl, heterocycloalkyl, heterocycloalkenyl, aryl, heteroaryl, cycloalkylalkyl, heterocycloalkylalkyl, arylalkyl, heteroarylalkyl, arylalkenyl, cycloalkylheteroalkyl, heterocycloalkylheteroalkyl, heteroarylheteroalkyl, arylheteroalkyl, hydroxy, hydroxyalkyl, alkoxy, alkoxyalkyl, alkoxyaryl, alkenyloxy, alkynyloxy, cycloalkylkoxy, heterocycloalkyloxy, aryloxy, heteroaryloxy, arylalkyloxy, amino, alkylamino, aminoalkyl, acylamino, arylamino, phenoxy, benzyloxy, COOH, COOR₄, SH, CONHR₄, NHR₄, —(CH₂)ₙNHCOR₄, NHCOR₄, NHCOOR₄ NHCONHR₄, C(═NOH)R₄, NHSOR₄ NHSO₂R₄, —(CH₂)ₙ—NR₆R₇, alkoxycarbonyl, alkylaminocarbonyl, sulfonyl, alkylsulfonyl, alkylsulfinyl, arylsulfonyl, arylsulfinyl, aminosulfonyl, aminosulfinyl, SR₄ and acyl each of which may optionally be substituted, provided that R₂ does not contain the moiety NHCONHCO or NHCONHSO₂;
R₃ is selected from the group consisting of H, halogen, alkyl, alkenyl, alkynyl, haloalkyl, haloalkenyl, heteroalkyl, cycloalkyl, cycloalkenyl, heterocycloalkyl, heterocycloalkenyl, aryl, heteroaryl, cycloalkylalkyl, heterocycloalkylalkyl, arylalkyl, heteroarylalkyl, arylalkenyl, cycloalkylheteroalkyl, heterocycloalkylheteroalkyl, heteroarylheteroalkyl, arylheteroalkyl, hydroxy, hydroxyalkyl, alkoxy, alkoxyalkyl, alkoxyaryl, alkenyloxy, alkynyloxy, cycloalkylkoxy, heterocycloalkyloxy, aryloxy, heteroaryloxy, arylalkyloxy, amino, alkylamino, aminoalkyl, acylamino, arylamino, phenoxy, benzyloxy, COOH, COOR₄, SH, CONHR₄, NHR₄, —(CH₂)ₙNHCOR₄, NHCOR₄, NHCOOR₄ NHCONHR₄, C(═NOH)R₄, NHSOR₄ NHSO₂R₄, —(CH₂)ₙ—NR₆R₇, alkoxycarbonyl, alkylaminocarbonyl, sulfonyl, alkylsulfonyl, alkylsulfinyl, arylsulfonyl, arylsulfinyl, aminosulfonyl, aminosulfinyl, SR₄ and acyl; each of which may optionally be substituted provided that R₃ does not contain the moiety NHCONHCO or NHCONHSO₂;
or R₂ and R₃ together with portion of ring B may form a non-aromatic ring fused to B;
X and Y are the same or different and are independently selected from the group consisting of H, halogen, —CN, —NO₂, —CF₃, —OCF₃, alkyl, alkenyl, alkynyl, haloalkyl, haloalkenyl, haloalkynyl, heteroalkyl, cycloalkyl, cycloalkenyl, heterocycloalkyl, heterocycloalkenyl, heteroaryl, hydroxy, hydroxyalkyl, alkoxy, alkoxyalkyl, alkoxyaryl, alkoxyheteroaryl, alkenyloxy, alkynyloxy, cycloalkyloxy, cycloalkenyloxy, heterocycloalkyloxy, heterocycloalkenyloxy, aryloxy, heteroaryloxy, arylalkyl, heteroarylalkyl, arylalkyloxy, amino, alkylamino, acylamino, aminoalkyl, arylamino, sulfonylamino, sulfinylamino, sulfonyl, alkylsulfonyl, arylsulfonyl, aminosulfonyl, aminoalkyl, alkoxyalky, —COOH, —C(O)OR₄, —COR₄, —SH, —SR₄, —OR₄, acyl and —NR₈R₉ each of which may be optionally substituted;
each R₄ is independently selected from the group consisting of H, alkyl, alkenyl, alkynyl, haloalkyl, heteroalkyl, cycloalkyl, heterocycloalkyl, aryl, heteroaryl, cycloalkylalkyl, heterocycloalkylalkyl, arylalkyl, heteroarylalkyl and acyl each of which may be optionally substituted;
each R₆ and R₇ is independently selected from the group consisting of H, alkyl, alkenyl, alkynyl, haloalkyl, heteroalkyl, cycloalkyl, heterocycloalkyl, aryl, heteroaryl, cycloalkylalkyl, heterocycloalkylalkyl, arylalkyl, heteroarylalkyl and acyl each of which may be optionally substituted;
each R₈ and R₉ is independently selected from the group consisting of H, alkyl, alkenyl, alkynyl, haloalkyl, heteroalkyl, cycloalkyl, heterocycloalkyl, aryl, heteroaryl, cycloalkylalkyl, heterocycloalkylalkyl, arylalkyl, heteroarylalkyl and acyl each of which may be optionally substituted;
n is an integer from 0 to 6,
m is an integer from 0 to 4;
   or a pharmaceutically acceptable salt or prodrug thereof.

A useful group of compounds within the scope of Formula (I) are those compounds of Formula (Ia) 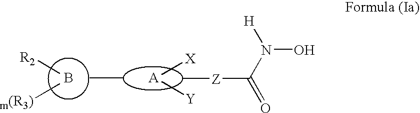
wherein
Z is a single bond or a C₁-C₄ hydrocarbon chain which may contain 0 to 1 double or triple bonds, unsubstituted or substituted with one or more substituents independently selected from the group consisting of C₁-C₄ alkyl;
A is an aromatic ring selected from the group consisting of optionally substituted arylene and optionally substituted heteroarylene, wherein A is not benzimidazole and when Z is a single bond then A is not selected from the group consisting of phenylene and six-membered heteroarylene containing 3 or less than 3 nitrogens;
B is an aromatic ring selected from the group consisting of optionally substituted aryl, optionally substituted arylene, optionally substituted heteroaryl and optionally substituted heteroarylene and wherein A and B can not both be phenylene and wherein
when Z is a single bond then B is not a bicyclic aryl or bicyclic heteroaryl;
wherein A and B are connected via a carbon-carbon bond;
R₂ is selected from C₁-C₁₀ alkyl, alkenyl, heteroalkyl, haloalkyl, alkynyl, aryl, cycloalkyl, heterocycloalkyl, heteroaryl, C₄-C₉ heterocycloalkylalkyl, cycloalkylalkyl (e.g., cyclopropylmethyl), arylalkyl (e.g. benzyl), heteroarylalkyl (e.g. pyridylmethyl), hydroxyl, hydroxyalkyl, alkoxy, amino, alkylamino, aminoalkyl, acylamino, phenoxy, alkoxyalkyl, benzyloxy, alkylsulfonyl, arylsulfonyl, aminosulfonyl, —C(O)OR₄, —C(O)OH, —SH, —CONHR₄, —NHCONHR₄, C(═NOH)R₄, —C(O)C(O)OR₄, C(O)CONHR₄, CON(R₅)OR₄, COCON(R₄)OR₄, NHCOR₄, and acyl; each of the above is unsubstituted or optionally substituted with one or more substituents independently selected from the group consisting of: halogen; ═O; ═S; —CN; and —NO₂; and alkyl, alkenyl, heteroalkyl, haloalkyl, alkynyl, aryl, cycloalkyl, heterocycloalkyl, heteroaryl, hydroxyl, hydroxyalkyl, alkoxy, alkylamino, aminoalkyl, acylamino, phenoxy, alkoxyalkyl, benzyloxy, alkylsulfonyl, arylsulfonyl, aminosulfonyl, —C(O)OR₅, —C(O)OH, —SH, —C(O)C(O)OR₅, C(O)CONHR₅, CON(R₅)OR₅, COCON(R₅)OR₅, NHCOR₅, and acyl; wherein R₂ does not contain the moiety NHCONHCO or NHCONHSO₂;
R₃ is selected from H, C₁-C₁₀ alkyl, alkenyl, heteroalkyl, haloalkyl, alkynyl, aryl, cycloalkyl, heterocycloalkyl, heteroaryl, C₄-C₉ heterocycloalkylalkyl, cycloalkylalkyl (e.g., cyclopropylmethyl), arylalkyl (e.g. benzyl), heteroarylalkyl (e.g. pyridylmethyl), hydroxyl, hydroxyalkyl, alkoxy, amino, alkylamino, aminoalkyl, acylamino, phenoxy, alkoxyalkyl, benzyloxy, alkylsulfonyl, arylsulfonyl, aminosulfonyl, —C(O)OR₄, —C(O)OH, —SH, —CONHR₄, —NHCONHR₄, C(═NOH)R₄, —C(O)C(O)OR₄, C(O)CONHR₄, CON(R₅)OR₄, COCON(R₄)OR₄, NHCOR₄, and acyl; each of the above is unsubstituted or optionally substituted with one or more substituents independently selected from the group consisting of: halogen; ═O; ═S; —CN; and —NO₂; and alkyl, alkenyl, heteroalkyl, haloalkyl, alkynyl, aryl, cycloalkyl, heterocycloalkyl, heteroaryl, hydroxyl, hydroxyalkyl, alkoxy, alkylamino, aminoalkyl, acylamino, phenoxy, alkoxyalkyl, benzyloxy, alkylsulfonyl, arylsulfonyl, aminosulfonyl, —C(O)OR₅, —C(O)OH, —SH, —C(O)C(O)OR₅, C(O)CONHR₅, CON(R₅)OR₅, COCON(R₅)OR₅, NHCOR₅, and acyl; wherein R₃ does not contain the moiety NHCONHCO or NHCONHSO₂;
   or R₂ and R₃ together with portion of ring B may form a non-aromatic ring fused to B.

X and Y are the same or different and independently selected from the group consisting of: H, halo, C₁-C₄ alkyl, such as CH₃ and CF₃, NO₂, OR₄, SR₄, C(O)R₅, CN, and NR₈ R₉;
R₄ is selected from H, C₁-C₄ alkyl, heteroalkyl, aryl, heteroaryl, acyl;
R is selected from H, C₁-C₄ alkyl;
R₈ and R₉ are the same or different and independently selected from the group consisting of H, C₁-C₆ alkyl, C₄-C₉ cycloalkyl, C₄-C₉ heterocycloalkyl, aryl, heteroaryl, arylalkyl, and heteroarylalkyl;
m is an integer from 0 to 4;
   or a pharmaceutically acceptable salt or prodrug thereof.

In further embodiments there are disclosed hydroxamate compounds of Formula (Ib): 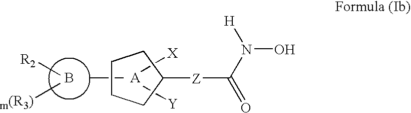
wherein
Z is a single bond or a C₁-C₄ hydrocarbon chain which may contain 0 to 1 double bond or triple bond, unsubstituted or substituted with one or more substituents independently selected from the group consisting of C₁-C₄ alkyl;
A is an optionally substituted five-membered heteroarylene;
B is an aromatic ring which is selected from the group consisting of optionally substituted aryl, optionally substituted arylene or optionally substituted heteroaryl or optionally substituted heteroarylene; wherein when Z is a single bond then B is not a bicyclic aryl or bicyclic heteroaryl;
wherein A and B are connected via a carbon-carbon bond;
R₂ is selected from the group consisting of halogen, alkyl, alkenyl, alkynyl, haloalkyl, haloalkenyl, heteroalkyl, cycloalkyl, cycloalkenyl, heterocycloalkyl, heterocycloalkenyl, aryl, heteroaryl, cycloalkylalkyl, heterocycloalkylalkyl, arylalkyl, heteroarylalkyl, arylalkenyl, cycloalkylheteroalkyl, heterocycloalkylheteroalkyl, heteroarylheteroalkyl, arylheteroalkyl, hydroxy, hydroxyalkyl, alkoxy, alkoxyalkyl, alkoxyaryl, alkenyloxy, alkynyloxy, cycloalkylkoxy, heterocycloalkyloxy, aryloxy, heteroaryloxy, arylalkyloxy, amino, alkylamino, aminoalkyl, acylamino, arylamino, phenoxy, benzyloxy, COOH, COOR₄, SH, CONHR₄, NHR₄, —(CH₂)ₙNHCOR₄, NHCOR₄, NHCOOR₄ NHCONHR₄, C(═NOH)R₄, NHSOR₄ NHSO₂R₄, —(CH₂)ₙ—NR₆R₇, alkoxycarbonyl, alkylaminocarbonyl, sulfonyl, alkylsulfonyl, alkylsulfinyl, arylsulfonyl, arylsulfinyl, aminosulfonyl, aminosulfinyl, SR₄ and acyl each of which may optionally be substituted, wherein R₂ does not contain the moiety NHCONHCO or NHCONHSO₂;
R₃ is selected from the group consisting of H, halogen, alkyl, alkenyl, alkynyl, haloalkyl, haloalkenyl, heteroalkyl, cycloalkyl, cycloalkenyl, heterocycloalkyl, heterocycloalkenyl, aryl, heteroaryl, cycloalkylalkyl, heterocycloalkylalkyl, arylalkyl, heteroarylalkyl, arylalkenyl, cycloalkylheteroalkyl, heterocycloalkylheteroalkyl, heteroarylheteroalkyl, arylheteroalkyl, hydroxy, hydroxyalkyl, alkoxy, alkoxyalkyl, alkoxyaryl, alkenyloxy, alkynyloxy, cycloalkylkoxy, heterocycloalkyloxy, aryloxy, heteroaryloxy, arylalkyloxy, amino, alkylamino, aminoalkyl, acylamino, arylamino, phenoxy, benzyloxy, COOH, COOR₄, SH, CONHR₄, NHR₄, —(CH₂)ₙNHCOR₄, NHCOR₄, NHCOOR₄ NHCONHR₄, C(═NOH)R₄, NHSOR₄ NHSO₂R₄, —(CH₂)ₙNR₆R₇, alkoxycarbonyl, alkylaminocarbonyl, sulfonyl, alkylsulfonyl, alkylsulfinyl, arylsulfonyl, arylsulfinyl, aminosulfonyl, aminosulfinyl, SR₄ and acyl; each of which may optionally be substituted wherein R₃ does not contain the moiety NHCONHCO or NHCONHSO₂;
X and Y are the same or different and are independently selected from the group consisting of H, halo, C₁-C₄ alkyl, such as CH₃ and CF₃, NO₂, OR₄, SR₄, C(O)Rs, CN, and NR₈ R₉.
R₄ is selected from H, C₁-C₄ alkyl, heteroalkyl, aryl, heteroaryl, acyl;
R₅ is selected from H, C₁-C₄ alkyl;
each R₆ and R₇ is independently selected from the group consisting of H, alkyl, alkenyl, alkynyl, haloalkyl, heteroalkyl, cycloalkyl, heterocycloalkyl, aryl, heteroaryl, cycloalkylalkyl, heterocycloalkylalkyl, arylalkyl, heteroarylalkyl and acyl each of which may be optionally substituted;
R₈ and R₉ are the same or different and are independently selected from the group consisting of H, C₁-C₆ alkyl, C₄-C₉ cycloalkyl, C₄-C₉ heterocycloalkyl, aryl, heteroaryl, arylalkyl, heteroarylalkyl;
n is an integer from 0 to 6;
m is an integer from 0 to 4;
   or a pharmaceutically acceptable salt or prodrug thereof.

In a particularly preferred embodiment of the compounds of Formula (Ib) the B moiety is attached to the 3rd or 4^{th} position relative to Z of ring A.

In yet a further embodiment of the compounds of Formula (I) there are disclosed compounds of the Formula (Ic): 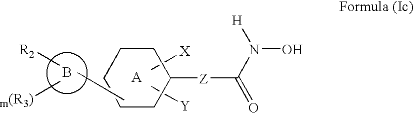
wherein
Z is a single bond or a C₁-C₄ hydrocarbon chain which may contain 0 to 1 double bond or triple bond, unsubstituted or substituted with one or more substituents independently selected from the group consisting of C₁-C₄ alkyl;
A is a six-membered aromatic ring which is selected from the group consisting of optionally substituted arylene or optionally substituted heteroarylene and when Z is a single bond then A is not selected from the group consisting of phenylene and six-membered heteroarylene containing 3 or less than 3 nitrogens;
B is an aromatic ring and is attached to the 3rd or 4^{th} position relative to Z of ring A selected from the group consisting of optionally substituted aryl, optionally substituted arylene, optionally substituted heteroaryl and optionally substituted heteroarylene and wherein A and B can not both be phenylene;
wherein A and B are connected via a carbon-carbon bond;
R₂ is selected from the group consisting of halogen, alkyl, alkenyl, alkynyl, haloalkyl, haloalkenyl, heteroalkyl, cycloalkyl, cycloalkenyl, heterocycloalkyl, heterocycloalkenyl, aryl, heteroaryl, cycloalkylalkyl, heterocycloalkylalkyl, arylalkyl, heteroarylalkyl, arylalkenyl, cycloalkylheteroalkyl, heterocycloalkylheteroalkyl, heteroarylheteroalkyl, arylheteroalkyl, hydroxy, hydroxyalkyl, alkoxy, alkoxyalkyl, alkoxyaryl, alkenyloxy, alkynyloxy, cycloalkylkoxy, heterocycloalkyloxy, aryloxy, heteroaryloxy, arylalkyloxy, amino, alkylamino, aminoalkyl, acylamino, arylamino, phenoxy, benzyloxy, COOH, COOR₄, SH, CONHR₄, NHR₄, —(CH₂)ₙNHCOR₄, NHCOR₄, NHCOOR₄ NHCONHR₄, C(═NOH)R₄, NHSOR₄ NHSO₂R₄, —(CH₂)ₙ—NR₆R₇, alkoxycarbonyl, alkylaminocarbonyl, sulfonyl, alkylsulfonyl, alkylsulfinyl, arylsulfonyl, arylsulfinyl, aminosulfonyl, aminosulfinyl, SR₄ and acyl each of which may optionally be substituted, wherein R₂ does not contain the moiety NHCONHCO or NHCONHSO₂;
R₃ is selected from the group consisting of H, halogen, alkyl, alkenyl, alkynyl, haloalkyl, haloalkenyl, heteroalkyl, cycloalkyl, cycloalkenyl, heterocycloalkyl, heterocycloalkenyl, aryl, heteroaryl, cycloalkylalkyl, heterocycloalkylalkyl, arylalkyl, heteroarylalkyl, arylalkenyl, cycloalkylheteroalkyl, heterocycloalkylheteroalkyl, heteroarylheteroalkyl, arylheteroalkyl, hydroxy, hydroxyalkyl, alkoxy, alkoxyalkyl, alkoxyaryl, alkenyloxy, alkynyloxy, cycloalkylkoxy, heterocycloalkyloxy, aryloxy, heteroaryloxy, arylalkyloxy, amino, alkylamino, aminoalkyl, acylamino, arylamino, phenoxy, benzyloxy, COOH, COOR₄, SH, CONHR₄, NHR₄, —(CH₂)ₙNHCOR₄, NHCOR₄, NHCOOR₄ NHCONHR₄, C(═NOH)R₄, NHSOR₄ NHSO₂R₄, —(CH₂)ₙNR₆R₇ alkoxycarbonyl, alkylaminocarbonyl, sulfonyl, alkylsulfonyl, alkylsulfinyl, arylsulfonyl, arylsulfinyl, aminosulfonyl, aminosulfinyl, SR₄ and acyl; each of which may optionally be substituted wherein R₃ does not contain the moiety NHCONHCO or NHCONHSO₂;
X and Y are the same or different and independently selected from H, halo, C₁-C₄ alkyl, such as CH₃ and CF₃, NO₂, OR₄, SR₄, C(O)R₅, CN, and NR₈ R₉;
R₄ is selected from H, C₁-C₄ alkyl, heteroalkyl, aryl, heteroaryl, acyl;
R₅ is selected from H, C₁-C₄ alkyl;
each R₆ and R₇ is independently selected from the group consisting of H, alkyl, alkenyl, alkynyl, haloalkyl, heteroalkyl, cycloalkyl, heterocycloalkyl, aryl, heteroaryl, cycloalkylalkyl, heterocycloalkylalkyl, arylalkyl, heteroarylalkyl and acyl each of which may be optionally substituted;
R₈ and R₉ are the same or different and independently selected from H, C₁-C₆ alkyl, C₄-C₉ cycloalkyl, C₄-C₉ heterocycloalkyl, aryl, heteroaryl, arylalkyl, heteroarylalkyl;
n is an integer from 0 to 6;
m is an integer from 0 to 4;
   or a pharmaceutically acceptable salt or prodrug thereof.

In a particularly preferred embodiment of the compounds of Formula (Ic) Z is CH₂ or CH═CH, A is a phenylene or six membered heteroarylene.

Another preferred compound is that of Formula (Id): 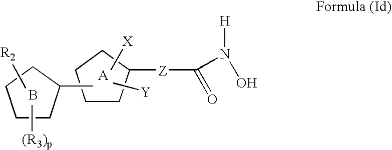
wherein 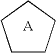
is selected from the group consisting of 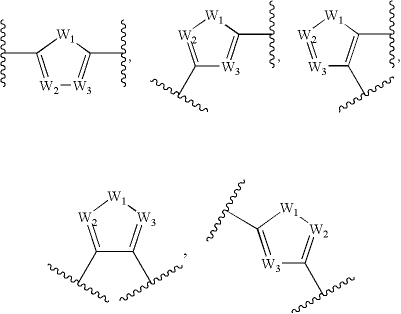
wherein W₁ is selected from the group consisting of O, S and NH;
W₂ and W₃ are independently selected from the group consisting of N, CX and CY;
p is an integer from 0 to 3;
   wherein Z, X, Y, B, R₂ and R₃ are as described above for formula (I), or a pharmaceutically acceptable salt or prodrug thereof.

In a preferred embodiment B is selected from the group consisting of: 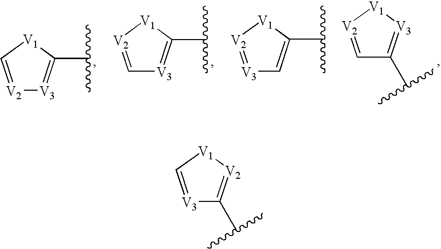
wherein V, is selected from the group consisting of O, S and NH;
V₂ and V₃ are selected from the group consisting of N, CR₂, and CR₃;
   wherein R₂ and R₃ are as described above.

In the embodiments discussed above A is preferably a group of formula: 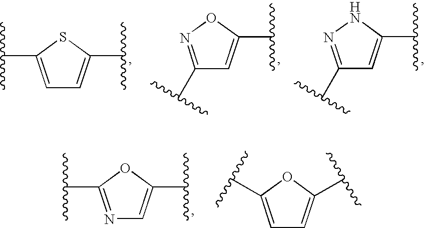
p is preferably 0 or 1, most preferably 0.

Another preferred compound is a compound of Formula (Ie): 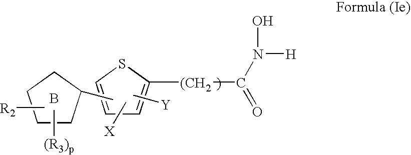
wherein B is a 5-membered heteroarylene, p is an integer from 0 to 3, and X, Y, R₂ and R₃ are as described for Formula (I). R₂ is preferably selected from the group consisting of:
—NH₂,
—(CH₂)ₙNHCOR₄,
—NHSO₂R₄,
—NR₄,
—(CH₂)ₙNR₆R₇,
-arylalkyl,
-heteroarylalkyl,
   each of which may be optionally substituted.

wherein n is an integer from 1 to 6, and R₄, R₆ and R₇ are as described for formula (I), or a pharmaceutically acceptable salt or prodrug thereof.

B is preferably a group of Formula: 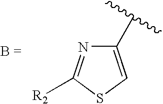
wherein R₂ is as described for formula (I).
p is preferably 0 or 1, most preferably 0.

Another preferred compound is a compound of Formula (If): 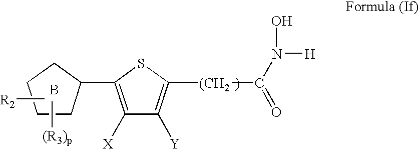
wherein B is a 5-membered heteroarylene, p is an integer from 0 to 3, and X, Y, R₂ and R₃ are as described for Formula (I). R₂ is preferably selected from the group consisting of:
—NH₂,
—(CH₂)ₙNHCOR₄,
—NHSO₂R₄,
—NR₄,
—(CH₂)ₙNR₆R₇.
-arylalkyl,
-heteroarylalkyl,
   each of which may be optionally substituted.

wherein n is an integer from 1 to 6, and R₄, R₆ and R₇ are as described for formula (I), or a pharmaceutically acceptable salt or prodrug thereof.

B is preferably a group of Formula: 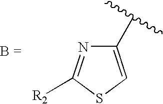
wherein R₂ is as described for formula (I).
p is preferably 0 or 1, most preferably 0.

In another preferred embodiment the invention provides compounds of Formula (Ig): 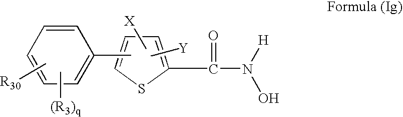
wherein q is an integer from 0 to 4, and X, Y, R₂ and R₃ are as described for Formula (I). R₃₀ is preferably selected from the group consisting of:
—NH₂,
—(CH₂)ₙNHCOR₄,
—NHSO₂R₄,
—(CH₂)ₙNR₆R₇.
-arylalkyl,
-heteroarylaikyl,
   each of which may be optionally substituted
   wherein n is an integer from 0 to 6 and R₄, R₆ and R₇ are as described for Formula (I), or a pharmaceutically acceptable salt or prodrug thereof.
   q is preferably 0 or 1, most preferably 0.

In another preferred embodiment the invention provides compounds of Formula (Ih): 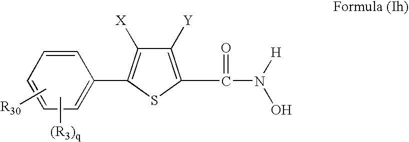
wherein q is an integer from 0 to 4, and X, Y, R₂ and R₃ are as described for Formula (I). R₃₀ is preferably selected from the group consisting of:
—NH₂,
—(CH₂)ₙNHCOR₄,
—NHSO₂R₄,
—NR₄,
—(CH₂)ₙNR₆R₇.
-arylalkyl,
-heteroarylalkyl,
   each of which may be optionally substituted
   wherein n is an integer from 0 to 6 and R₄, R₆ and R₇ are as described for Formula (I), or a pharmaceutically acceptable salt or prodrug thereof.
   q is preferably 0 or 1, most preferably 0.

In another preferred embodiment the invention provides a compound of Formula (Ii): 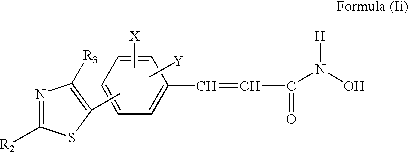
wherein X, Y, R₂ and R₃ are as described for Formula (I). R₂ is preferably selected from the group consisting of:
—NH₂,
—(CH₂)ₙNHCOR₄,
—NHSO₂R₄,
—NR₄,
—(CH2)ₙNR₆R₇.
-arylalkyl,
-heteroarylalkyl,
   each of which may be optionally substituted.
   where n is is an integer from 0 to 6 and R₄, R₆ and R₇ are as described in Formula (I), or a pharmaceutically acceptable salt or prodrug thereof.

In another embodiment the compounds are of Formula (I;): 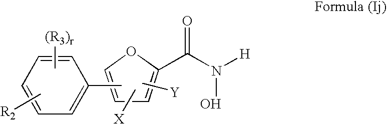
wherein r is an integer from 0 to 4, and X, Y, R₂ and R₃ are as described for Formula (I). R₂ is preferably selected from the group consisting of:
—NH₂,
—(CH₂)ₙNHCOR₄,
—NHSO₂R₄,
—NR₄
—(CH₂)ₙNR₆R₇.
-arylalkyl,
-heteroarylalkyl,
   each of which may be optionally substituted
   wherein n=is an integer from 0 to 6, R₄, R₆ and R₇ are the same as in Formula (I), or a pharmaceutically acceptable salt or prodrug thereof.
   r is preferably 0 or 1, most preferably 0.

In another embodiment the compounds are of Formula (Ik): 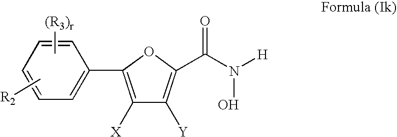
wherein r is an integer from 0 to 4, and X, Y, R₂ and R₃ are as described for Formula (I). R₂ is preferably selected from the group consisting of:
—NH₂,
—(CH₂)ₙNHCOR₄,
—NHSO₂R₄,
—NR₄,
—(CH₂)ₙNR₆R₇.
-arylalkyl,
-heteroarylalkyl,
   each of which may be optionally substituted
   wherein n=is an integer from 0 to 6, R₄, R₆ and R₇ are the same as in Formula (I), or a pharmaceutically acceptable salt or prodrug thereof.
   r is preferably 0 or 1, most preferably 0.

As with any group of structurally related compounds which possess a particular utility, certain groups are preferred for the compounds of the invention in their end use application.

The Z moiety is preferably a single bond, a group of formula CH₂ or a group of formula —CH═CH—. When Z is a group of formula —CH═CH— the moiety is preferably in the “E” configuration.

It is preferred that when Z is a single bond then A is not 2,5-thiophenylene.

In one embodiment of the invention it is preferred that R₂ and R₃ are selected from the group consisting of H, C₁-C₁₀ alkyl, alkenyl, heteroalkyl, haloalkyl, alkynyl, aryl, cycloalkyl, heterocycloalkyl, heteroaryl, C₄-C₉ heterocycloalkylalkyl, cycloalkylalkyl (e.g., cyclopropylmethyl), arylalkyl (e.g. benzyl), heteroarylalkyl (e.g. pyridylmethyl), hydroxyl, hydroxyalkyl, alkoxy, amino, alkylamino, aminoalkyl, acylamino, phenoxy, alkoxyalkyl, benzyloxy, alkylsulfonyl, arylsulfonyl, aminosulfonyl, —C(O)OR₄, —C(O)OH, —SH, —CONHR₄, —NHCONHR₄, C(═NOH)R₄, and acyl.

In another preferred embodiment it is preferred that R₃ is H and R₂ is selected from the group consisting of NH₂, —(CH₂)ₙNHCOR₄, NHSO₂R₄, (CH₂)ₙNR₄, (CH₂)ₙNR₆R₇, NR₆R₇ arylalkyl, heteroarylalkyl, arylheteroalkyl, heteroarylheteroalky;l, halogen, and alkoxy, each of which may be optionally substituted, wherein n is 0, 1 or 2, and R₄, R₆ and R₇ are as defined herein.

It is particularly preferred that R₂ is a group of formula —(CH₂)ₙ—NR₆R₇ wherein n is 0 and R₆ and R₇ are independently selected from the group consisting of H, cyclopropyl, 2-(4-Hydroxy-3,5-dimethoxy-phenyl)-ethyl, 3-Pyrrolidin-1-yl-propyl, 2-Morpholin-4-yl-ethyl, 3-Morpholin-4-yl-propyl, 2-Dimethylamino-ethyl. 4-[4-(2,3-Dimethyl-phenyl)-piperazin-1-ylmethyl, 3-Imidazol-1-yl-propyl, 3-phenyl-propyl, (2-Hydroxy-ethyl)-phenethyl, 2-Hydroxy-ethyl-2-(1H-indol-3-yl)-ethyl, (2-Morpholin-4-yl-ethyl)-phenethyl, 2-(2-methyl-1H-indol-3-yl)-ethyl, 2-(1H-indol-3-yl)-ethyl, pyridin-3-ylmethyl, 3-hydroxy-propyl, 2-pyridin-2-yl-ethyl, 2-pyridin-3-yl-ethyl, pyridin-3-ylmethyl, 2-pyridin-4-yl-ethyl, benzyl, 3-phenyl-propyl, 2-phenoxy-ethyl, morpholin-4-yl, pyridin-2-yl, phenethyl, 2-(4-bromo-phenyl)-ethyl, 2-(4-fluoro-phenyl)-ethyl, 3-imidazol-1-yl-propyl, 2-(1H-imidazol-4-yl)-ethyl, 1H-Benzoimidazol-2-ylmethyl, 2-piperidin-1-yl-ethyl, 2-pyrrolidin-1-yl-ethyl, 2-cyclohex-1-enyl-ethyl, 2-ethyl-hexyl, 2-thiophen-2-yl-ethyl, 3,3-diphenyl-propyl, 2-biphenyl4-yl-ethyl, 4-phenoxy-phenyl, 2-(3-phenoxy-phenyl)-ethyl, 2-(2,3-dimethoxy-phenyl, 2-(2,4-dichloro-phenyl)-ethyl, cyclohexylmethyl, hexyl, isobutyl, 3-isopropoxy-propyl, 2-phenoxy-ethyl, 2-isopropoxy-ethyl, 3-methoxy-benzyl, 4-[1,2,3]thiadiazol4-yl-benzyl, 2,4-dichloro-benzyl, 2-(2-methoxy-phenyl)-ethyl, 2-(3-fluoro-phenyl)-ethyl, 2-(2-fluoro-phenyl)-ethyl, 2,2-diphenyl-ethyl, 2-(4-methoxy-phenyl)-ethyl, 2-(3-chloro-phenyl)-ethyl, 4-phenyl-butyl, 3-phenyl-propyl, 3,3-diphenyl-propyl, 3-(4-methyl-piperazin-1-yl, 3-morpholin4-yl-propyl, 3-(2-oxo-pyrrolidin-1-yl)-propyl, 3-pyrrolidin-1-yl-propyl, tetrahydro-furan-2-ylmethyl, 2-diethylamino-ethyl, 2-dimethylamino-ethyl.

If R₂ or R₃ are substituted particularly preferred substituents are selected from the group consisting of halogen, ═O, ═S, —CN, —NO₂, alkyl, alkenyl, heteroalkyl, haloalkyl, alkynyl, aryl, cycloalkyl, heterocycloalkyl, heteroaryl, hydroxy, hydroxyalkyl, alkoxy, alkylamino, aminoalkyl, acylamino, phenoxy, alkoxyalkyl, benzyloxy, alkylsulfonyl, arylsulfonyl, aminosulfonyl, —C(O)OR₅, COOH, SH, —C(O)C(O)OR₅, C(O)CONHR₅, CON(R₅)OR₅, COCON(R₅)OR₅, NHCOR₅ and acyl; such that neither R₂ nor R₃ contains an acylurea unit (NHCONHCO) or sulfonylurea unit [NHCONHS(O)₂]
X and Y are preferably selected from the group consisting of H, halo, C₁-C₄ alkyl, such as CH₃ and CF₃, NO₂, OR₄, SR₄, C(O)R₅, CN, and NR₈ R₉, most preferably H.
R₄ is preferably selected from the group consisting of H, C₁-C₄ alkyl, cycloalkyl, heteroalkyl, aryl, heteroaryl, and acyl.
R₅ is preferably H, C₁-C₄ alkyl or cycloalkyl;
R₆ and R₇ are the same or different and are preferably selected from the group consisting of H, C₁-C₆ alkyl, C₄-C₉ cycloalkyl, C₄-C₉ heterocycloalkyl, aryl, heteroaryl, arylalkyl and heteroarylalkyl. R₈ and R₉ are the same or different and are preferably selected from the group consisting of H, C₁-C₆ alkyl, C₄-C₉ cycloalkyl, C₄-C₉ heterocycloalkyl, aryl, heteroaryl, arylalkyl and heteroarylalkyl.

In one preferred embodiment A is an optionally substituted 5-membered heteroarylene ring. In this embodiment it is preferred that A is selected from the group consisting of 2,5-furanylene; 2,4-furanylene; 2,3-furanylene; 3,4-furanylene; 2,5-thiophenylene; 2,4-thiophenylene, 2,3-thiophenylene; 3,4-thiophenylene; 1,2-pyrrolylene; 1,3-pyrrolylene; 1,4-pyrrolylene; 1,5-pyrrolylene; 2,3-pyrrolylene; 2,4-pyrrolylene; 2,5-pyrrolylene; 3,4-pyrrolylene; 2,5-oxazolylene; 2,4-oxazolylene; 4,5-oxazolylene, 2,5-thiazolylene; 2,4-thiazolylene; 4,5-thiazolylene 1,2-imidazolylene; 1,4-imidazolylene; 1,5-imidazolylene; 2,4-imidazolylene; 2,5- imidazolylene; 4,5-imidazolylene 1,3-pyrazolylene; 1,4-pyrazolylene; 1,5-pyrazolylene; 3,4-pyrazolylene; 3,5-pyrazolylene; 4,5-pyrazolylene; 3,4-isoxazolylene; 3,5-isoxazolylene; 4,5-isoxazolylene; 3,4-isothiazolylene; 3,5-isothiazolylene; 4,5-isothiazolylene; 4,5-(1,2,3-oxadiazoly)-ene; 3,5, -(1,2,4-oxadiazolyl)ene; 1,4-(1,2,3-triazolyl)ene; 1,5-(1,2,3-triazolyl)ene; 4,5-(1,2,3-triazolyl)ene; 1,3-(1,2,4-triazolyl)ene; 1,5-(1,2,4-tdazolyl)ene; 3,5-(1,2,4-triazolyl)ene; 3,5-(1,2,4-thiadiazolyl)ene; 2,5-(1,3,4-thiadiazolyl)ene, and 1,5-tetrazolylene.

It is particularly preferred that A is selected from the group consisting of 2,5-thiophenylene; 3,5-isoxazolylene; 3,5-pyrazolylene; 2,5-oxazolyiene; 3,5-pyrazolylene; 2,5-furanylene and 2,4-thiophenylene.

When A is a five-membered heteroarylene it is preferred that B is attached to the 3^{rd} or 4^{th} position relative to Z of Ring A.

In another preferred embodiment A is an optionally substituted phenylene or an optionally substituted 6-membered heteroarylene. It is preferred that when A is phenylene then B is not a 5-membered heteroaryl or 5-membered heteroaryiene.

In another preferred embodiment B is an optionally substituted 5-membered heteroarylene. In this embodiment it is preferred that B is selected from the group consisting of 2,5-furanylene; 2,4-furanylene; 2,3-furanylene; 3,4-furanylene; 2,5-thiophenylene; 2,4-thiophenylene, 2,3-thiophenylene; 3,4-thiophenylene; 1,2-pyrrolylene; 1,3-pyrrolylene; 1,4-pyrrolylene; 1,5-pyrrolylene; 2,3-pyrrolylene; 2,4-pyrrolylene; 2,5-pyrrolylene; 3,4-pyrrolylene; 2,5-oxazolylene; 2,4-oxazolylene; 4,5-oxazolylene, 2,5-thiazolylene; 2,4-thiazolylene; 4,5-thiazolylene 1,2-imidazolylene; 1,4-imidazolylene; 1,5-imidazolylene; 2,4-imidazolylene; 2,5-imidazolylene; 4,5-imidazolylene 1,3-pyrazolylene; 1,4-pyrazolylene; 1,5-pyrazolylene; 3,4-pyrazolylene; 3,5-pyrazolylene; 4,5-pyrazolylene; 3,4-isoxazolylene; 3,5-isoxazolylene; 4,5-isoxazolylene; 3,4-isothiazolylene; 3,5-isothiazolylene; 4,5-isothiazolylene; 4,5-(1,2,3-oxadiazoly)-ene; 3,5, -(1,2,4-oxadiazolyl)ene; 1,4-(1,2,3-triazolyl)ene; 1,5-(1,2,3-triazolyl)ene; 4,5-(1,2,3-triazolyl)ene; 1,3-(1,2,4-triazolyl)ene; 1,5-(1,2,4-triazolyl)ene; 3,5-(1,2,4-triazolyl)ene; 3,5-(1,2,4-thiadiazolyl)ene; 2,5-(1,3,4-thiadiazolyl)ene, and 1,5-tetrazolylene.

It is particularly preferred that B is an optionally substituted 5-membered heteroarylene selected from the group consisting of 2,4-thiazolylene; 4,2-thiazolylene; 1,3-phenylene;

2,5-thiophenylene and 1,4-phenylene.

It is particularly preferred that both A and B are 5-membered heteroarylene rings.

It is preferred that B is not a bicyclic heteroaryl or bicyclic heteroarylene having 9 ring atoms. It is also preferred that B is not a monocyclic, bicyclic or polycyclic heteroarylene substituted by a cycloheteroalky moiety. With reference to the compounds above when any moieties are said to be optionally substituted it is preferred that If they are substituted with one or more substituents then the substituents are independently selected from the group consisting of halogen, ═O, ═S, —CN, —NO₂, —CF₃, —OCF₃, alkyl, alkenyl, alkynyl, haloalkyl, haloalkenyl, haloalkynyl, heteroalkyl, cycloalkyl, cycloalkenyl, heterocycloalkyl, heterocycloalkenyl, aryl, heteroaryl, hydroxy, hydroxyalkyl, alkoxy, alkoxyalkyl, alkoxyaryl, alkoxyheteroaryl, alkenyloxy, alkynyloxy, cycloalkyloxy, cycloalkenyloxy, heterocycloalkyloxy, heterocycloalkenyloxy, aryloxy, heteroaryloxy, arylalkyl, heteroarylalkyl, cycloalkylalkyl, heterocycloalkylalkyl, arylalkyloxy, amino, alkylamino, acylamino, aminoalkyl, arylamino, sulfonylamino, sulfinylamino, sulfonyl, aikylsulfonyl, arylsulfonyl, aminosulfonyl, aminoalkyl, alkoxyalky, CH₂heterocycloalkylCOOR₁₀ heterocycloalkylCOOR₁₀, —COOH, —COR₅, —C(O)OR₅, CONHR₅, —C(O)C(O)OR₅, C(O)CONHR₅, CON(R₅)OR₅, COCON(R₅)OR₅, NHCOR₅, CH₂NCOOR₁₀, NHCOOR₅, NHCONHR₅, C(═NOH)R₅, —SH, —SR₅, —OR₅ and acyl;
wherein R₁₀ is selected from H, alkyl, acyl and aryl.
n is preferably 0, 1 or 2, more preferably 0 or 1.
m is preferably 0, 1 or 2, more preferably 0 or 1, most preferably 1.

In another embodiment it is preferred that when A is a thiazolylene, benzothiazolylene, oxazolylene or benzoxazolylene, B is not a phenyl or substituted phenyl which is attached to position 2 of the ring.

In another embodiment it is preferred that when A is 2,5-oxazolene and Z is single bond, R₂=R₃=H, then B is not a phenyl, 4-Cl-phenyl, 4-CH₃O-phenyl or 4-NO₂-phenyl.

In addition to compounds of as described above, certain embodiments disclosed are also directed to pharmaceutically acceptable salts, pharmaceutically acceptable prodrugs, and pharmaceutically active metabolites of such compounds, and pharmaceutically acceptable salts of such metabolites. Such compounds, salts, prodrugs and metabolites are at times collectively referred to herein as “HDAC inhibiting agents” or “HDAC inhibitors”. In certain embodiments the compounds disclosed are used to modify deacetylase activity, in some cases histone deacetylase activity and in some cases HDAC 8, or HDAC 1 activity.

Certain embodiments disclosed also relate to pharmaceutical compositions each comprising a therapeutically effective amount of a HDAC inhibiting agent of the embodiments described and optionally comprising a pharmaceutically acceptable carrier or diluent for treating cellular proliferative ailments. The term “effective amount” as used herein indicates an amount of compound necessary to administer to a host to achieve a therapeutic result, e.g., inhibition of proliferation of malignant cancer cells, benign tumor cells or other proliferative cells.

The invention also relates to pharmaceutical compositions including a compound of the invention with a pharmaceutically acceptable carrier, diluent or excipient.

In yet a further aspect the present invention provides a method of treatment of a disorder caused by, associated with or accompanied by disruptions of cell proliferation and/or angiogenesis including administration of a therapeutically effective amount of a compound of Formula (I).

The method preferably includes administration of a compound of Formula (Ia), more preferably a compound of Formula (Ib), even more preferably a compound of Formula (Ic) or a compound of Formula (Id), most preferably a compound of Formula (Ie) to (Ik) as described herein.

The disorder is preferably selected from the group consisting of but not limited to cancer (e.g. breast cancer, colon cancer, prostate cancer, pancreatic cancer, leukemias, lymphomas), inflammatory diseases/immune system disorders, angiofibroma, cardiovascular diseases (e.g. restenosis, arteriosclerosis), fibrotic diseases (e.g. liver fibrosis), diabetes, autoimmune diseases, chronic and acute neurodegenerative disease like disruptions of nerval tissue, Huntington's disease and infectious diseases like fungal, bacterial and viral infections. In another embodiment the disorder is a proliferative disorder. The proliferative disorder is preferably cancer. The cancer can include solid tumors or hematologic malignancies.

The invention also provides agents for the treatment of a disorder caused by, associated with or accompanied by disruptions of cell proliferation and/or angiogenesis including a compound of Formula (I) as disclosed herein. The agent is preferably an ant-cancer agent.

The agent preferably contains a compound of Formula (Ia), more preferably a compound of Formula (Ib), even more preferably a compound of Formula (Ic) or a compound of Formula (Id), most preferably a compound of Formula (Ie) to (Ik) as described herein.

The invention also relates to the use of compounds of Formula (I) in the preparation of a medicament for the treatment of a disorder caused by, associated with or accompanied by disruptions of cell proliferation and/or angiogenesis. The disorder is preferably a proliferative disorder, most preferably a cancer.

The compounds of the present invention surprisingly show low toxicity, together with a potent anti-proliferative activity.

In yet a further embodiment the invention provides a method of treatment of a disorder that can be treated by the inhibition of histone deacetylase including administration of a therapeutically effective amount of a compound of Formula (I).

In yet a further embodiment the invention provides a method of treatment of a disorder, disease or condition that are mediated by deacetylase activity such as histone deacetylase including administration of a therapeutically effective amount of a compound of Formula (I).

The method preferably includes administration of a compound of Formula (Ia), more preferably a compound of Formula (Ib) even more preferably a compound of Formula (Ic) or a compound of Formula (Id), most preferably a compound of Formula (Ie) to (Ik) as described herein.

The disorder is preferably selected from the group consisting of but not limited to Proliferative disorders (e.g. cancer); Neurodegenerative diseases including Huntington's Disease, Polyglutamine diseases, Parkinson's Disease, Alzheimer's Disease, Seizures, Striatonigral degeneration, Progressive supranuclear palsy, Torsion dystonia, Spasmodic torticollis and dyskinesis, Familial tremor, Gilles de la Tourette syndrome, Diffuse Lewy body disease, Progressive supranuclear palsy, Pick's disease, Intracerebral haemorrhage Primary lateral sclerosis, Spinal muscular atrophy, Amyotrophic lateral sclerosis, Hypertrophic interstitial polyneuropathy, Retinitis pigmentosa, Hereditary optic atrophy, Hereditary spastic paraplegia, Progressive ataxia and Shy-Drager syndrome; Metabolic diseases including Type 2 diabetes; Degenerative Diseases of the Eye including Glaucoma, Age-related macular degeneration, Rubeotic glaucoma, Intersitital keratitis, Diabetic retinopathy; Inflammatory diseases and/or Immune system disorders including Rheumatoid Arthritis (RA), Osteoarthritis, Juvenile chronic arthritis, Graft versus Host disease, Psoriasis, Asthma, Spondyloarthropathy, Crohn's Disease, inflammatory bowel disease, Colitis Ulcerosa, Alcoholic hepatitis, Diabetes , Sjoegrens's syndrome, Multiple Sclerosis, Ankylosing spondylitis, Membranous glomerulopathy, Discogenic pain, Systemic Lupus Erythematosus; Disease involving angiogenesis including cancer, psoriasis, rheumatoid arthritis; Psychological disorders including bipolar disease, schizophrenia, depression and dementia; Cardiovascular Diseases including Heart failure, restenosis and arteriosclerosis; Fibrotic diseases including liver fibrosis, cystic fibrosis and angiofibroma; Infectious diseases including Fungal infections, such as Candida Albicans, Bacterial infections, Viral infections, such as Herpes Simplex, Protozoal infections, such as Malaria, Leishmania infection, Trypanosoma brucei infection, Toxoplasmosis and coccidiosis and Haematopoietic disorders including thalassemia, anemia and sickle cell anemia.

The invention also provides agents for the treatment of a disorder, disease or condition that can be treated by the inhibition of histone deacetylase including a compound of Formula (I) as disclosed herein. The agent is preferably an anti-cancer agent.

The invention also relates to the use of compounds of Formula (I) in the preparation of a medicament for the treatment of a disorder, disease or condition that can be treated by the inhibition of histone deacetylase.

The invention also provides a method for inhibiting cell proliferation including administration of an effective amount of a compound according to Formula (I).

In yet an even further aspect the invention provides a method of treatment of a neurodegenerative disorder In a patient including administration of a therapeutically effective amount of a compound of Formula (I). The method preferably includes administration of a compound of Formula (Ia), more preferably a compound of Formula (Ib) even more preferably a compound of Formula (Ic) or a compound of Formula (Id), most preferably a compound of (Ie) to (Ik) as described herein. The neurodegenerative disorder is preferably Huntington's Disease.

The invention also provides agents for the treatment of neurodegenerative disorder including a compound of Formula (I) as disclosed herein. The agent is preferably anti-Huntington's disease agent.

The invention also relates to the use of compounds of Formula (I) in the preparation of a medicament for the treatment of a neurodegenerative disorder. The neurodegenerative disorder is preferably Huntington's Disease.

In yet an even further aspect the invention provides a method of treatment of an inflammatory disease and/or immune system disorder in a patient including administration of a therapeutically effective amount of a compound of Formula (I). The method preferably includes administration of a compound of Formula (Ia), more preferably a compound of Formula (Ib) as described herein, even more preferably (Ic) or (Id), most preferably a compound of Formula (Ie) to (Ik). In one embodiment the inflammatory disease and/or immune system disorder is rheumatoid arthritis. In another embodiment the inflammatory disease and/or immune system disorder is Systemic Lupus Erythematosus.

The invention also provides agents for the treatment of inflammatory disease and/or immune system disorder including a compound of Formula (I) as disclosed herein.

The invention also relates to the use of compounds of Formula (I) in the preparation of a medicament for the treatment of inflammatory disease and/or immune system disorder. In one embodiment the inflammatory disease and/or immune system disorder is rheumatoid arthritis. In another embodiment the inflammatory disease and/or immune system disorder is Systemic Lupus Erythematosus.

In another embodiment the present invention provides the use of a compound of Formula (I) to modify deacetylase activity, preferably histone deacetylase activity, even more preferably HDACI or HDAC8.

The invention also provides the use of a compound of Formula (I) to treat cancer. In another embodiment, the cancer is selected from a group including but not limited to breast cancer, lung cancer, ovarian cancer, prostate cancer, head and neck cancer, renal cancer, gastric cancer, colon cancer, pancreatic cancer and brain cancer.

In a further aspect the invention provides a method of treatment of a hematological malignancy including administration of a compound of Formula (Ia), more preferably a compound of Formula (Ib) as described herein, even more preferably (Ic) or (Id), most preferably a compound of Formula (Ie) to (Ik).

The invention also provides use of a compound of Formula (I) in the preparation of a medicament for the treatment of a hematologic malignancy. The hematologic malignancy is preferably selected from the group consisting of B-cell lymphoma, T-cell lymphoma and leukemia.

In a further aspect the invenbon provides a method of treatment of a solid tumor including administration of an effective amount of a compound of Formula (I). The method preferably includes administration of a compound of Formula (Ia), more preferably a compound of Formula (Ib) as described herein, even more preferably (Ic) or (Id), most preferably a compound of Formula (Ie) to (Ik).

The invention also provides the use of compounds of Formula (I) in the preparation of a medicament to treat solid tumors. The solid tumor is preferably selected from the group consisting of breast cancer, lung cancer, ovarian cancer, prostate cancer, head and neck cancer, renal cancer, gastic cancer, colon cancer, pancreatic cancer and brain cancer.

A method of induction of apoptosis of tumor cells including contacting the tumor cell with an effective amount of a compound of Formula (I). The method preferably includes administration of a compound of Formula (Ia), more preferably a compound of Formula (Ib) as described herein, even more preferably (Ic) or (Id), most preferably a compound of Formula (Ie) to (Ik).

The invention also provides the use of a compound of Formula (I) in the preparation of a medicament for the induction of cell death such as apoptosis of tumor cells.

### DETAILED DESCRIPTION OF THE INVENTION

There are disclosed hydroxamate compounds, for example biaryl compounds containing hydroxamic acid in one of the substituents, that may be inhibitors of deacetylases, including but not limited to inhibitors of histone deacetylases. The hydroxamate compounds may be suitable for prevention or treatment of a disorder caused by, associated with or accompanied by disruptions of cell proliferation and/or angiogenesis when used either alone or together with a pharmaceutically acceptable carrier, diluent or excipient. An example of such a disorder is cancer.

As used herein the term ‘cancer’ is a general term intended to encompass the vast number of conditions that are characterised by uncontrolled abnormal growth of cells.

It is anticipated that the compounds of the invention will be useful in treating various cancers including but not limited to bone cancers including Ewing's sarcoma, osteosarcoma, chondrosarcoma and the like, brain and CNS tumors including acoustic neuroma, neuroblastomas, glioma and other brain tumors, spinal cord tumors, breast cancers, colorectal cancers, colon cancers, advanced colorectal adenocarcinomas, endocrine cancers including adenocortical carcinoma, pancreatic cancer, pituitary cancer, thyroid cancer, parathyroid cancer, thymus cancer, multiple endocrine neoplasma, gastrointestinal cancers including stomach cancer, esophageal cancer, small intestine cancer, Liver cancer, extra hepatic bile duct cancer, gastrointestinal carcinoid tumor, gall bladder cancer, genitourinary cancers including testicular cancer, penile cancer, prostate cancer, gynaecological cancers including cervical cancer, ovarian cancer, vaginal cancer, uterus/endometrium cancer, vulva cancer, gestational trophoblastic cancer, fallopian tube cancer, uterine sarcoma, head and neck cancers including oral cavity cancer, lip cancer, salivary gland cancer, larynx cancer, hypopharynx cancer, orthopharynx cancer, nasal cancer, paranasal cancer, nasopharynx cancer, leukemias including childhood leukemia, acute lymphocytic leukemia, acute myeloid leukemia, chronic lymphocytic leukemia, chronic myeloid leukemia, hairy cell leukernia, acute promyelocytic leukemia, plasma cell leukemia, myelomas, haematological disorders including myelodysplastic syndromes, myeloproliferative disorders, aplastic anemia, Fanconi anemia, Waldenstroms Macroglobulinemia, lung cancers including small cell lung cancer, non-small cell lung cancer, lymphomas including Hodgkin's disease, non-Hodgkin's lymphoma, cutaneous T-cell lymphoma, peripheral T-cell lymphorna, AIDS related Lymphoma, B-cell lymphoma, Burkitt's lymphoma, eye cancers including retinoblastoma, intraocular melanoma, skin cancers including melanoma, non-melanorna skin cancer, merkel cell cancer, soft tissue sarcomas such as childhood soft tissue sarcoma, adult soft tissue sarcoma, Kaposi's sarcoma, urinary system cancers including kidney cancer, Wilms tumor, bladder cancer, urethral cancer, and transitional cell cancer.

Preferred cancers that may be treated by the compounds of the present invention include but are not limited to breast cancer, lung cancer, ovarian cancer, prostate cancer, head and neck cancer, renal cancer, gastric cancer, colon cancer, pancreatic cancer and brain cancer.

Preferred cancers that may be treated by compounds of the present invention include but are not limited to B-cell lymphoma (e.g. Burkitt's lymphoma), leukemias (e.g. Acute promyelocytic leukemia), cutaneous T-cell lymphoma (CTCL) and peripheral T-cell lymphoma.

Preferred cancers that may be treated by compounds of the present invention include but are not limited to solid tumors and hematologic malignancies.

The compounds may also be used in the treatment of a disorder involving, relating to, or associated with dysregulation of histone deacetylase (HDAC).

There are a number of disorders that have been implicated by or known to be mediated at least in part by HDAC activity, where HDAC activity is known to play a role in triggering disease onset, or whose symptoms are known or have been shown to be alleviated by HDAC inhibitors. Disorders of this type that would be expected to be amenable to treatment with the compounds of the invention include the following but not limited to:

Proliferative disorders (e.g. cancer); Neurodegenerative diseases including Huntington's Disease, Polyglutamine diseases, Parkinson's Disease, Alzheimer's Disease, Seizures, Striatonigral degeneration, Progressive supranuclear palsy, Torsion dystonia, Spasmodic torticollis and dyskinesis, Familial tremor, Gilles de la Tourette syndrome, Diffuse Lewy body disease, Progressive supranuclear palsy, Pick's disease, intracerebreal haemorrphage, Primary lateral sclerosis, Spinal muscular atrophy, Amyotrophic lateral sclerosis, Hypertrophic interstitial polyneuropathy, Retinitis pigmentosa, Hereditary optic atrophy, Hereditary spastic paraplegia, Progressive ataxia and Shy-Drager syndrome; Metabolic diseases including Type 2 diabetes; Degenerative Diseases of the Eye including Glaucoma, Age-related macular degeneration, Rubeotic glaucoma, Intersitital keratitis, Diabetic retinopathy; Inflammatory diseases and/or Immune system disorders including Rheumatoid Arthritis (RA), Osteoarthritis, Juvenile chronic arthritis, Graft versus Host disease, Psoriasis, Asthma, Spondyloarthropathy, Crohn's Disease, inflammatory bowel disease Colits Ulcerosa, Alcoholic hepatitis, Diabetes, Sjoegrens's syndrome, Multiple Sclerosis, Ankylosing spondylitis, Membranous glomerulopathy, Discogenic pain, Systemic Lupus Erythematosus; Disease involving angiogenesis including cancer, psoriasis, rheumatoid arthritis; Psychological disorders including bipolar disease, schizophrenia, mainia, depression and dementia; Cardiovascular Diseases including heart failure, restenosis and arteriosclerosis; Fibrotic diseases including liver fibrosis, cystic fibrosis and angiofibroma; Infectious diseases including Fungal infections, such as Candida Albicans, Bacterial Infections, Viral infections, such as Herpes Simplex, Protozoal infections, such as Malaria, Leishmania infection, Trypanosoma brucei infection, Toxoplasmosis and coccidiosis and Haematopoietic disorders including thalassemia, anemia and sickle cell anemia.

The hydroxamate compounds of the present invention have the following structure (I): 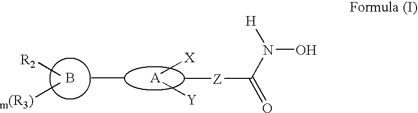
wherein
Z is a single bond or a C₁-C₄ hydrocarbon chain containing no more than 1 double or triple bond, optionally substituted with one or mnore substituents independently selected from the group consisting of C₁-C₄ alkyl;
A is an aromatic ring selected from the group consisting of optionally substituted arylene and optionally substituted heteroarylene, wherein A is not benzimidazole and when Z is a single bond then A is not selected frorn the group consisting of phenylene and six-membered heteroarylene containing 3 or less than 3 nitrogens;
B is an aromatic ring selected from the group consisting of optionally substituted aryl, optionally substituted arylene, optionally substituted heteroaryl and optionally substituted heteroarylene and wherein A and B can not both be phenylene and wherein when Z is a single bond then B is not a bicyclic aryl or bicyclic heteroaryl;
wherein A and B are connected via a carbon-carbon bond;
R₂ is selected from the group consisting of halogen, alkyl, alkenyl, alkynyl, haloalkyl, haloalkenyl, heteroalkyl, cycloalkyl, cycloalkenyl, heterocycloalkyl, heterocycloalkenyl, aryl, heteroaryl, cycloalkylalkyl, heterocycloalkylalkyl, arylalkyl, heteroarylalkyl, arylalkenyl, cycloalkylheteroalkyl, heterocycloalkylheteroalkyl, heteroarylheteroalkyl, arylheteroalkyl, hydroxy, hydroxyalkyl, alkoxy, alkoxyalkyl, alkoxyaryl, alkenyloxy, alkynyloxy, cycloalkylkoxy, heterocycloalkyloxy, aryloxy, heteroaryloxy, arylalkyloxy, amino, alkylamino, aminoalkyl, acylamino, arylamino, phenoxy, benzyloxy, COOH, COOR₄, SH, CONHR₄, NHR₄, —(CH₂)ₙNHCOR₄, NHCOR₄, NHCOOR₄ NHCONHR₄, C(═NOH)R₄, NHSOR₄ NHSO₂R₄, —(CH₂)ₙ—NR₆R₇, alkoxycarbonyl, alkylaminocarbonyl, sulfonyl, alkylsulfonyl, alkylsulfinyl, arylsulfonyl, arylsulfinyl, aminosulfonyl, aminosulfinyl, SR₄ and acyl each of which may optionally be substituted, provided that R₂ does not contain the moiety NHCONHCO or NHCONHSO₂;
R₃ is selected from the group consisting of H, halogen, alkyl, alkenyl, alkynyl, haloalkyl, haloalkenyl, heteroalkyl, cycloalkyl, cycloalkenyl, heterocycloalkyl, heterocycloalkenyl, aryl, heteroaryl, cycloalkylalkyl, heterocycloalkylalkyl, arylalkyl, heteroarylalkyl, arylalkenyl, cycloalkylheteroalkyl, heterocycloalkylheteroalkyl, heteroarylheteroalkyl, arylheteroalkyl, hydroxy, hydroxyalkyl, alkoxy, alkoxyalkyl, alkoxyaryl, alkenyloxy, alkynyloxy, cycloalkylkoxy, heterocycloalkyloxy, aryloxy, heteroaryloxy, arylalkyloxy, amino, alkylamino, aminoalkyl, acylamino, arylamino, phenoxy, benzyloxy, COOH, COOR₄, SH, CONHR₄, NHR₄, —(CH₂)ₙNHCOR₄, NHCOR₄, NHCOOR₄ NHCONHR₄, C(═NOH)R₄, NHSOR₄ NHSO₂R₄, —(CH₂)ₙ—NR₆R₇ alkoxycarbonyl, alkylaminocarbonyl, sulfonyl, alkylsulfonyl, alkylsulfinyl, arylsulfonyl, arylsulfinyl, aminosulfonyl, aminosulfinyl, SR₄ and acyl; each of which may optionally be substituted provided that R₃ does not contain the moiety NHCONHCO or NHCONHSO₂;
or R₂ and R₃ together with portion of ring B may form a non-aromatic ring fused to B;
X and Y are the same or different and are independently selected from the group consisting of H, halogen, —CN, —NO₂, —CF₃, —OCF₃, alkyl, alkenyl, alkynyl, haloalkyl, haloalkenyl, haloalkynyl, heteroalkyl, cycloalkyl, cycloalkenyl, heterocycloalkyl, heterocycloalkenyl, heteroaryl, hydroxy, hydroxyalkyl, alkoxy, alkoxyalkyl, alkoxyaryl, alkoxyheteroaryl, alkenyloxy, alkynyloxy, cycloalkyloxy, cycloalkenyloxy, heterocycloalkyloxy, heterocycloalkenyloxy, aryloxy, heteroaryloxy, arylalkyl, heteroarylalkyl, arylalkyloxy, amino, alkylamino, acylamino, aminoalkyl, arylamino, sulfonylamino, sulfinylamino, sulfonyl, alkylsulfonyl, arylsulfonyl, aminosulfonyl, aminoalkyl, alkoxyalky, —COOH, —C(O)OR₄, —COR₄, —SH, —SR₄, —OR₄, acyl and —NR₈R₉ each of which may be optionally substituted;
each R₄ is independently selected from the group consisting of H, alkyl, alkenyl, alkynyl, haloalkyl, heteroalkyl, cycloalkyl, heterocycloalkyl, aryl, heteroaryl, cycloalkylalkyl, heterocycloalkylalkyl, arylalkyl, heteroarylalkyl and acyl each of which may be optionally substituted;
each R₆ and R₇ is independently selected from the group consisting of H, alkyl, alkenyl, alkynyl, haloalkyl, heteroalkyl, cycloalkyl, heterocycloalkyl, aryl, heteroaryl, cycloalkylalkyl, heterocycloalkylalkyl, arylalkyl, heteroarylalkyl and acyl each of which may be optionally substituted;
each R₈ and R₉ is independently selected from the group consisting of H, alkyl, alkenyl, alkynyl, haloalkyl, heteroalkyl, cycloalkyl, heterocycloalkyl, aryl, heteroaryl, cycloalkylalkyl, heterocycloalkylalkyl, arylalkyl, heteroarylalkyl and acyl each of which may be optionally substituted;
n is an integer from 0 to 6,
m is an integer from 0 to 4;
   or a pharmaceutically acceptable salt or prodrug thereof.

A useful group of compounds within the scope of Formula (I) are those compounds of Formula (Ia) 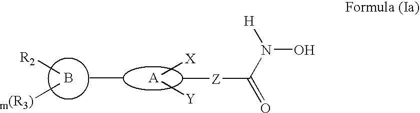
wherein
Z is a single bond or a C₁-C₄ hydrocarbon chain which may contain 0 to 1 double or triple bonds, unsubstituted or substituted with one or more substituents independently 9 selected from the group consisting of C₁-C₄ alkyl;
A is an aromatic ring selected from the group consisting of optionally substituted arylene and optionally substituted heteroarylene, wherein A is not benzimidazole and when Z is a single bond then A is not selected from the group consisting of phenylene and six-membered heteroarylene containing 3 or less than 3 nitrogens;
B is an aromatic ring selected from the group consisting of optionally substituted aryl, optionally substituted arylene, optionally substituted heteroaryl and optionally substituted heteroarylene and wherein A and B can not both be phenylene and wherein when Z is a single bond then B is not a bicyclic aryl or bicyclic heteroaryl;
wherein A and B are connected via a carbon-carbon bond;
R₂ is selected from C₁-C₁₀ alkyl, alkenyl, heteroalkyl, haloalkyl, alkynyl, aryl, cycloalkyl, heterocycloalkyl, heteroaryl, C₄-C₉ heterocycloalkylalkyl, cycloalkylalkyl (e.g., cyclopropylmethyl), arylalkyl (e.g. benzyl), heteroarylalkyl (e.g. pyridylmethyl), hydroxyl, hydroxyalkyl, alkoxy, amino, alkylamino, aminoalkyl, acylamino, phenoxy, alkoxyalkyl, benzyloxy, alkylsulfonyl, arylsulfonyl, aminosulfonyl, —C(O)OR₄, —C(O)OH, —SH, —CONHR₄, —NHCONHR₄, C(═NOH)R₄, —C(O)C(O)OR₄, C(O)CONHR₄, CON(R₅)OR₄, COCON(R₄)OR₄, NHCOR₄, and acyl; each of the above is unsubstituted or optionally substituted with one or more substituents independently selected from the group consisting of: halogen; ═O; ═S; —CN; and —NO₂; and alkyl, alkenyl, heteroalkyl, haloalkyl, alkynyl, aryl, cycloalkyl, heterocycloalkyl, heteroaryl, hydroxyl, hydroxyalkyl, alkoxy, alkylamino, aminoalkyl, acylamino, phenoxy, alkoxyalkyl, benzyloxy, alkylsulfonyl, arylsulfonyl, aminosulfonyl, —C(O)OR₅, —C(O)OH, —SH, —C(O)C(O)OR₅, C(O)CONHR₅, CON(R₅)OR₅, COCON(R₅)OR₅, NHCOR₅, and acyl; wherein R₂ does not contain the moiety NHCONHCO or NHCONHSO₂;
R₃ is selected from H, C₁-C₁₀ alkyl, alkenyl, heteroalkyl, haloalkyl, alkynyl, aryl, cycloalkyl, heterocycloalkyl, heteroaryl, C₄-C₉ heterocycloalkylalkyl, cycloalkylalkyl (e.g., cyclopropylmethyl), arylalkyl (e.g. benzyl), heteroarylalkyl (e.g. pyridylmethyl), hydroxyl, hydroxyalkyl, alkoxy, amino, alkylamino, aminoalkyl, acylamino, phenoxy, alkoxyalkyl, benzyloxy, alkylsulfonyl, arylsulfonyl, aminosulfonyl, —C(O)OR₄, —C(O)OH, —SH, —CONHR₄, —NHCONHR₄, C(═NOH)R₄, —C(O)C(O)OR₄, C(O)CONHR₄, CON(R₅)OR₄, COCON(R₄)OR₄, NHCOR₄, and acyl; each of the above is unsubstituted or optionally substituted with one or more substituents independently selected from the group consisting of: halogen; ═O; ═S; —CN; and —NO₂; and alkyl, alkenyl, heteroalkyl, haloalkyl, alkynyl, aryl, cycloalkyl, heterocycloalkyl, heteroaryl, hydroxyl, hydroxyalkyl, alkoxy, alkylamino, aminoalkyl, acylamino, phenoxy, alkoxyalkyl, benzyloxy, alkylsulfonyl, arylsulfonyl, aminosulfonyl, —C(O)OR₅, —C(O)OH, —SH, —C(O)C(O)OR₅, C(O)CONHR₅, CON(R₅)OR₅, COCON(RN)OR₅, NHCOR₅, and acyl; wherein R₃ does not contain the moiety NHCONHCO or NHCONHSO₂;
or R₂ and R₃ together with portion of ring B may form a non-aromatic ring fused to B;
X and Y are the same or different and independently selected from the group consisting of: H, halo, C₁-C₄ alkyl, such as CH₃ and CF₃, NO₂, OR₄, SR₄, C(O)R₅, CN, and NR₈ R₉;
R₄ is selected from H, C₁-C₄ alkyl, heteroalkyl, aryl, heteroaryl, acyl;
R₅ is selected from H, C₁-C₄ alkyl;
R₈ and R₉ are the same or different and independently selected from the group consisting of H, C₁-C₆ alkyl, C₄-C9 cycloalkyl, C₄-C₉ heterocycloalkyl, aryl, heteroaryl, arylalkyl, and heteroarylalkyl;
m is an integer from 0 to 4;
   or a pharmaceutically acceptable salt or prodrug thereof.

In further embodiments there are disclosed hydroxamate compounds of Formula (Ib): 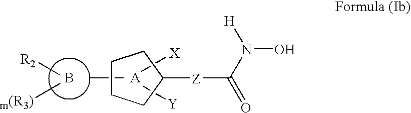
wherein
Z is a single bond or a C₁-C₄ hydrocarbon chain which may contain 0 to 1 double bond or triple bond, unsubstituted or substituted with one or more substituents independently selected from the group consisting of C₁-C₄ alkyl;
A is an optionally substituted five-membered heteroarylene;
B is an aromatic ring which is selected from the group consisting of optionally substituted aryl, optionally substituted arylene or optionally substituted heteroaryl or optionally substituted heteroarylene; wherein when Z is a single bond then B is not a bicyclic aryl or bicyclic heteroaryl;
wherein A and B are connected via a carbon-carbon bond;
R₂ is selected from the group consisting of halogen, alkyl, alkenyl, alkynyl, haloalkyl, haloalkenyl, heteroalkyl, cycloalkyl, cycloalkenyl, heterocycloalkyl, heterocycloalkenyl, aryl, heteroaryl, cycloalkylalkyl, heterocycloalkylalkyl, arylalkyl, heteroarylalkyl, arylalkenyl, cycloalkylheteroalkyl, heterocycloalkylheteroalkyl, heteroarylheteroalkyl, arylheteroalkyl, hydroxy, hydroxyalkyl, alkoxy, alkoxyalkyl, alkoxyaryl, alkenyloxy, alkynyloxy, cycloalkylkoxy, heterocycloalkyloxy, aryloxy, heteroaryloxy, arylalkyloxy, amino, alkylamino, aminoalkyl, acylamino, arylamino, phenoxy, benzyloxy, COOH, COOR₄, SH, CONHR₄, NHR₄, —(CH₂)ₙNHCOR₄, NHCOR₄, NHCOOR₄ NHCONHR₄, C(═NOH)R₄, NHSOR₄ NHSO₂R₄, —(CH₂)ₙ—NR₆R₇, alkoxycarbonyl, alkylaminocarbonyl, sulfonyl, alkylsulfonyl, alkylsulfinyl, arylsulfonyl, arylsulfinyl, aminosulfonyl, aminosulfinyl, SR₄ and acyl each of which rmnay optionally be substituted, wherein R₂ does not contain the moiety NHCONHCO or NHCONHSO₂;
R₃ is selected from the group consisting of H, halogen, alkyl, alkenyl, alkynyl, haloalkyl, haloalkenyl, heteroalkyl, cycloalkyl, cycloalkenyl, heterocycloalkyl, heterocycloalkenyl, aryl, heteroaryl, cycloalkylalkyl, heterocycloalkylalkyl, arylalkyl, heteroarylalkyl, arylalkenyl, cycloalkylheteroalkyl, heterocycloalkylheteroalkyl, heteroarylheteroalkyl, arylheteroalkyl, hydroxy, hydroxyalkyl, alkoxy, alkoxyalkyl, alkoxyaryl, alkenyloxy, alkynyloxy, cycloalkylkoxy, h eterocycloalkyloxy, aryloxy, heteroaryloxy, arylalkyloxy, amino, alkylamino, aminoalkyl, acylamino, arylamino, phenoxy, benzyloxy, COOH, COOR₄, SH, CONHR₄, NHR₄, —(CH₂)ₙNHCOR₄, NHCOR₄, NHCOOR₄ NHCONHR₄, C(═NOH)R₄, NHSOR₄ NHSO₂R₄, —(CH₂)ₙNR₆R₇ alkoxycarbonyl, alkylaminocarbonyl, sulfonyl, alkylsulfonyl, alkylsulfinyl, arylsulfonyl, arylsulfinyl, aminosulfonyl, aminosulfinyl, SR₄ and acyl; each of which rnay optionally be substituted wherein R₃ does not contain the moiety NHCONHCO or NHCONHSO₂;
X and Y are the same or different and are independently selected from the group consisting of H, halo, C₁-C₄ alkyl, such as CH₃ and CF₃, NO₂, OR₄, SR₄, C(O)R₅, CN, and NR₈ R₉,
R₄ is selected from H, C₁-C₄ alkyl, heteroalkyl, aryl, heteroaryl, acyl;
R₅ is selected from H, C₁-C₄ alkyl;
each R₆ and R₇ is independently selected from the group consisting of H, alkyl, alkenyl, alkynyl, haloalkyl, heteroalkyl, cycloalkyl, heterocycloalkyl, aryl, heteroaryl, cycloalkylalkyl, heterocycloalkylalkyl, arylalkyl, heteroarylalkyl and acyl each of which may be optionally substituted;
R₈ and R₉ are the same or different and are independently selected from the group consisting of H, C₁-C₆ alkyl, C₄-C₉ cycloalkyl, C₄-C₉ heterocycloalkyl, aryl, heteroaryl, arylalkyl, heteroarylalkyl;
n is an integer from 0 to 6;
m is an integer from 0 to 4;
   or a pharmaceutically acceptable salt or prodrug thereof.

In a particularly preferred embodiment of the compounds of Formula (Ib) the B moiety is attached to the 3rd or 4^{th} position relative to Z of ring A.

In yet a further embodiment of the compounds of Formula (I) there are disclosed compounds of the Formula (Ic): 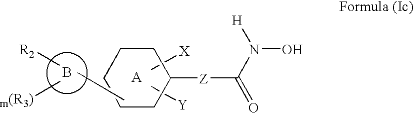
wherein
Z is a single bond or a C₁-C₄ hydrocarbon chain which may contain 0 to 1 double bond or triple bond, unsubsbtuted or substituted with one or more substituents independently selected from the group consisting of C₁-C₄ alkyl;

A is a six-membered aromatic ring which is selected frorm the group consisting of optionally substituted arylene or optionally substituted heteroarylene and when Z is a single bond then A is not selected from the group consisting of phenylene and six-membered heteroarylene containing 3 or less than 3 nitrogens;
B is an aromatic ring and is attached to the 3rd or 4^{th} position relative to Z of ring A selected from the group consisting of optionally substituted aryl, optionally substituted arylene, optionally substituted heteroaryl and optionally substituted heteroarylene and wherein A and B can not both be phenylene;
wherein A and B are connected via a carbon-carbon bond;
R₂ is selected from the group consisting of halogen, alkyl, alkenyl, alkynyl, haloalkyl, haloalkenyl, heteroalkyl, cycloalkyl, cycloalkenyl, heterocycloalkyl, heterocycloalkenyl, aryl, heteroaryl, cycloalkylalkyl, heterocycloalkylalkyl, arylalkyl, heteroarylalkyl, arylalkenyl, cycloalkylheteroalkyl, heterocycloalkylheteroalkyl, heteroarylheteroalkyl, arylheteroalkyl, hydroxy, hydroxyalkyl, alkoxy, alkoxyalkyl, alkoxyaryl, alkenyloxy, alkynyloxy, cycloalkylkoxy, heterocycloalkyloxy, aryloxy, heteroaryloxy, arylalkyloxy, amino, alkylamino, aminoalkyl, acylamino, arylamino, phenoxy, benzyloxy, COOH, COOR₄, SH, CONHR₄, NHR₄, —(CH₂)ₙNHCOR₄, NHCOR₄, NHCOOR₄ NHCONHR₄, C(═NOH)R₄, NHSOR₄ NHSO₂R₄, —(CH₂)ₙ—NR₆R₇, alkoxycarbonyl, alkylaminocarbonyl, sulfonyl, alkylsulfonyl, alkylsulfinyl, arylsulfonyl, arylsulfinyl, aminosulfonyl, aminosulfinyl, SR₄ and acyl each of which may optionally be substituted, wherein R₂ does not contain the moiety NHCONHCO or NHCONHSO₂;
R₃ is selected from the group consisting of H, halogen, alkyl, alkenyl, alkynyl, haloalkyl, haloalkenyl, heteroalkyl, cycloalkyl, cycloalkenyl, heterocycloalkyl, heterocycloalkenyl, aryl, heteroaryl, cycloalkylalkyl, heterocycloalkylalkyl, arylalkyl, heteroarylalkyl, arylalkenyl, cycloalkylheteroalkyl, heterocycloalkylheteroalkyl, heteroarylheteroalkyl, arylheteroalkyl, hydroxy, hydroxyalkyl, alkoxy, alkoxyalkyl, alkoxyaryl, al kenyloxy, alkynyloxy, cycloalkylkoxy, heterocycloalkyloxy, aryloxy, heteroaryloxy, arylalkyloxy, amino, alkylamino, aminoalkyl, acylamino, arylamino, phenoxy, benzyloxy, COOH, COOR₄, SH, CONHR₄, NHR₄, —(CH₂)ₙNHCOR₄, NHCOR₄, NHCOOR₄ NHCONHR₄, C(═NOH)R₄, NHSOR₄ NHSO₂R₄, —(CH₂)ₙNR₆R₇. alkoxycarbonyl, alkylaminocarbonyl, sulfonyl, alkylsulfonyl, alkylsulfinyl, arylsulfonyl, arylsulfinyl, aminosulfonyl, aminosulfinyl, SR₄ and acyl; each of which may optionally be substituted wherein R₃ does not contain the moiety NHCONHCO or NHCONHSO₂;
X and Y are the same or different and independently selected from H, halo, C₁-C₄ alkyl, such as CH₃ and CF₃, NO₂, OR₄, SR₄, C(O)R₅, CN, and NR₈ R₉;
R₄ is selected from H, C₁-C₄ alkyl, heteroalkyl, aryl, heteroaryl, acyl;
R₅ is selected from H, C₁-C₄ alkyl;
each R₆ and R₇ is independently selected from the group consisting of H, alkyl, alkenyl, alkynyl, haloalkyl, heteroalkyl, cycloalkyl, heterocycloalkyl, aryl, heteroaryl, cycloalkylalkyl, heterocycloalkylalkyl, arylalkyl, heteroarylalkyl and acyl each of which may be optionally substituted;
R₈ and R₉ are the same or different and independently selected from H, C₁-C₆ alkyl, C₄-C₉ cycloalkyl, C₄-C₉ heterocycloalkyl, aryl, heteroaryl, arylalkyl, heteroarylalkyl;
n is an integer from 0 to 6;

m is an integer from 0 to 4;

or a pharmaceutically acceptable salt or prodrug thereof.

In particular embodiments the compound is selected from compounds, and their pharmaceutically acceptable salts, selected from the group consisting of

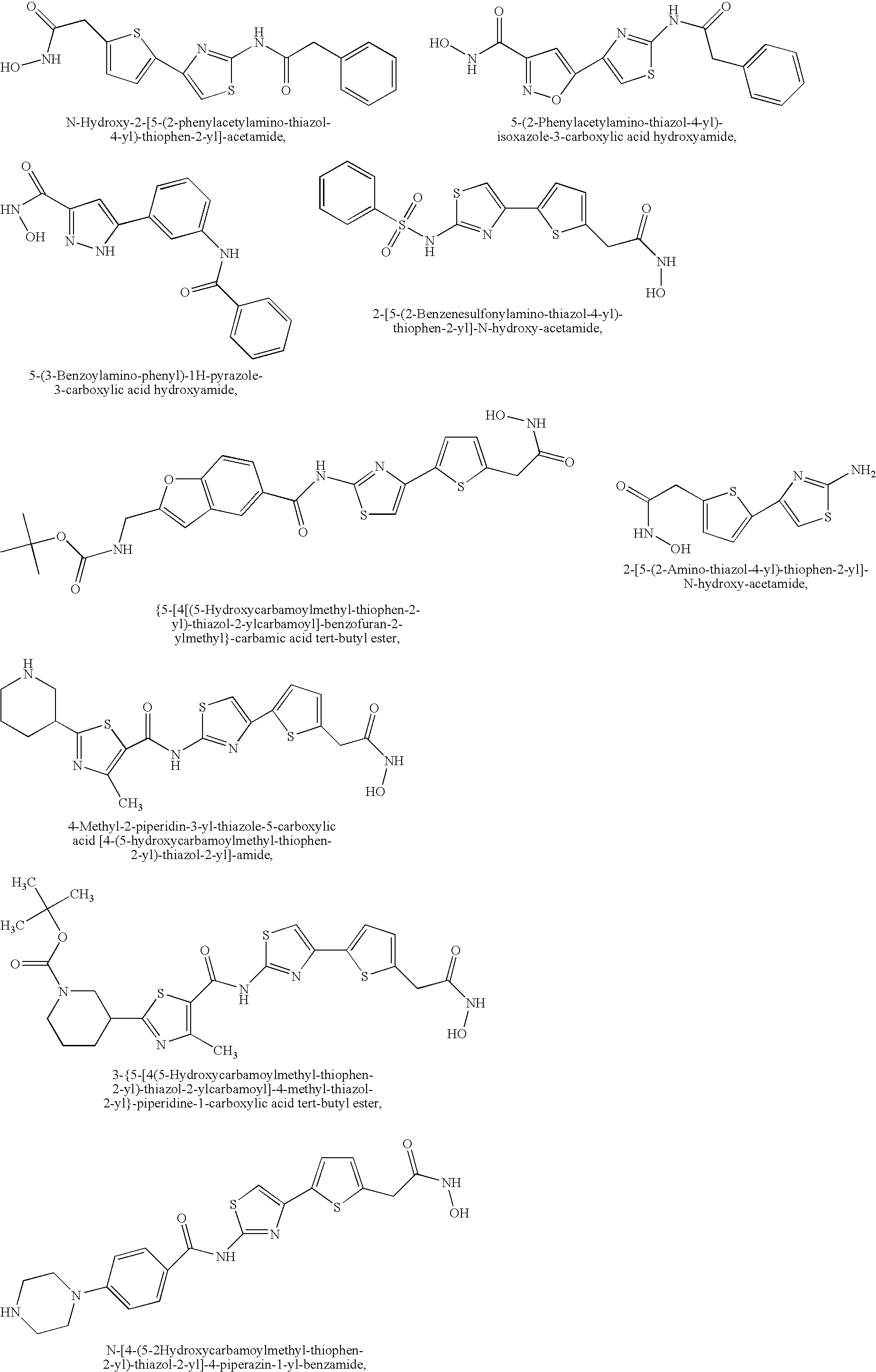

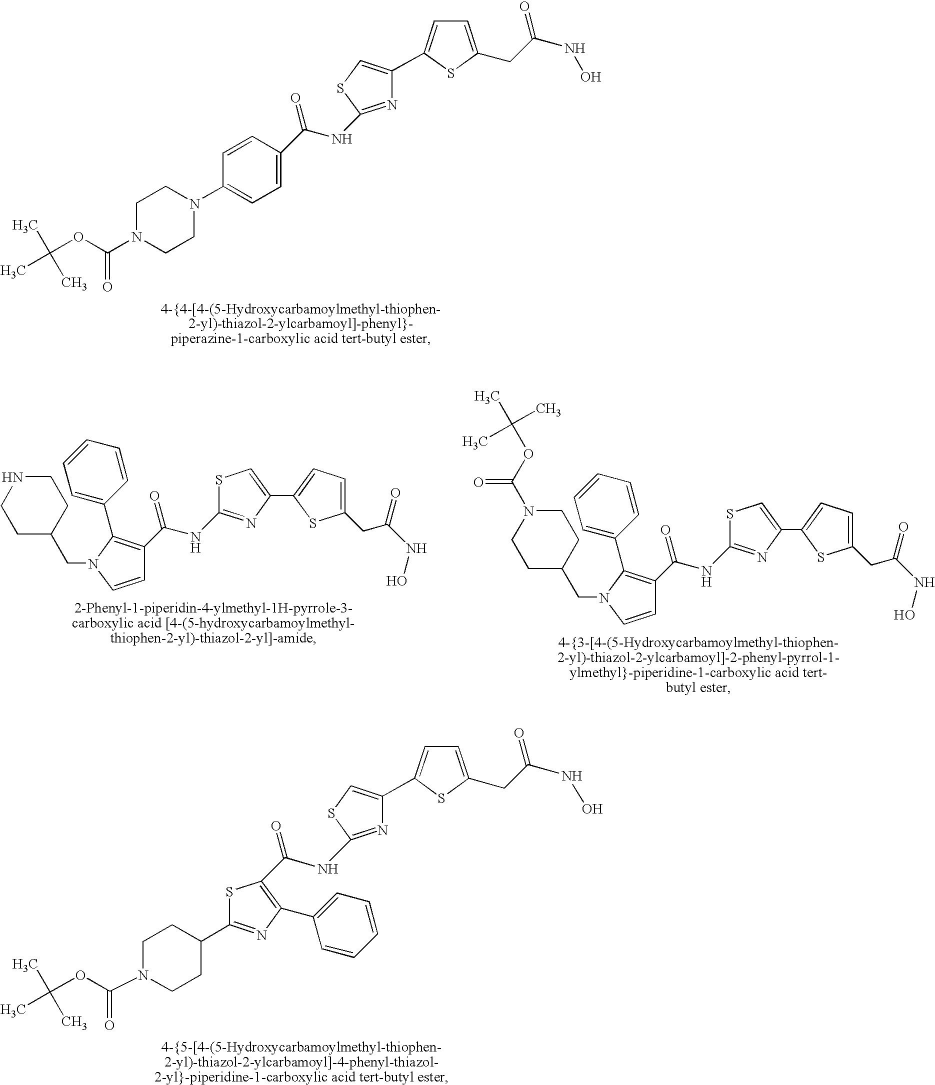

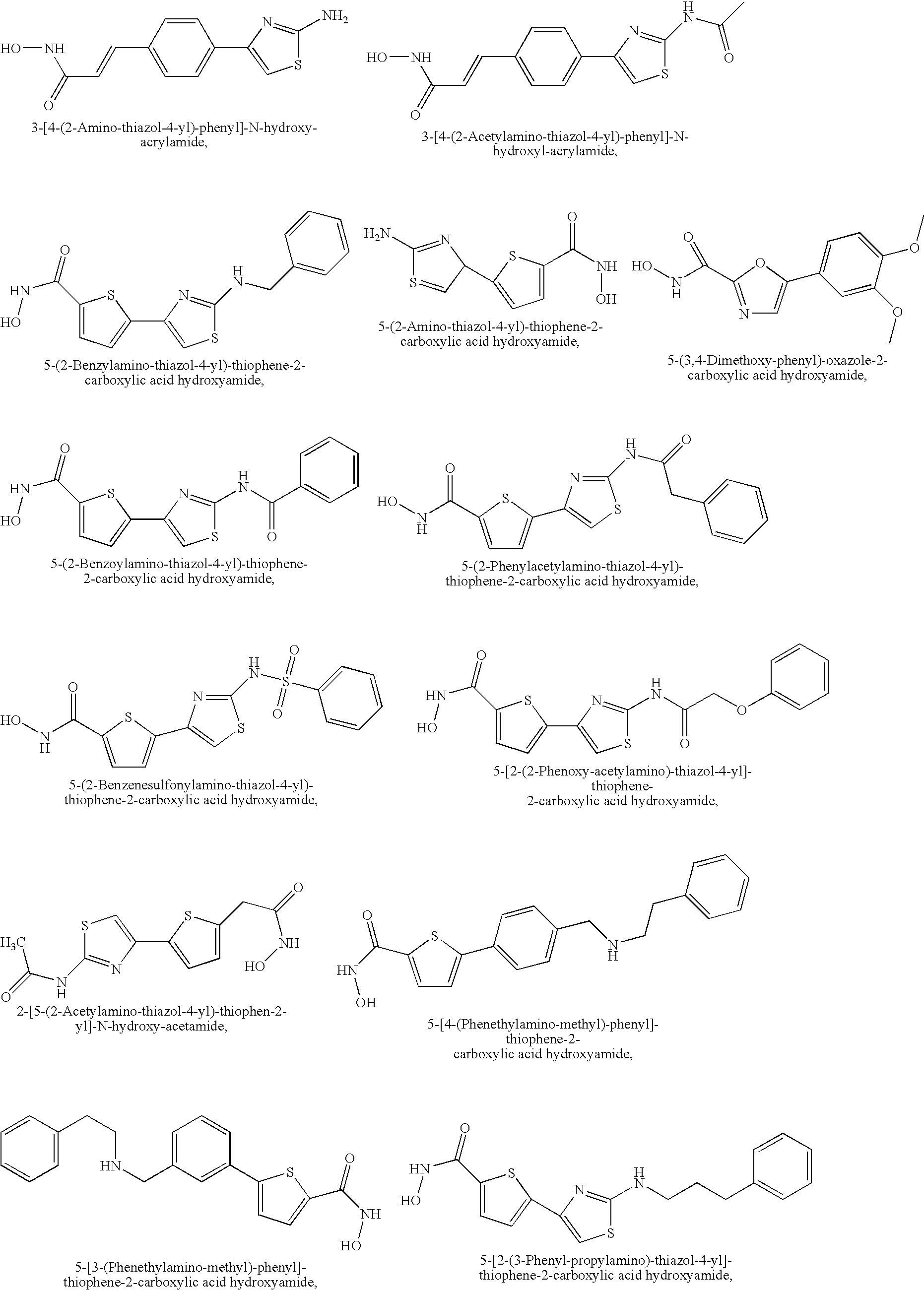

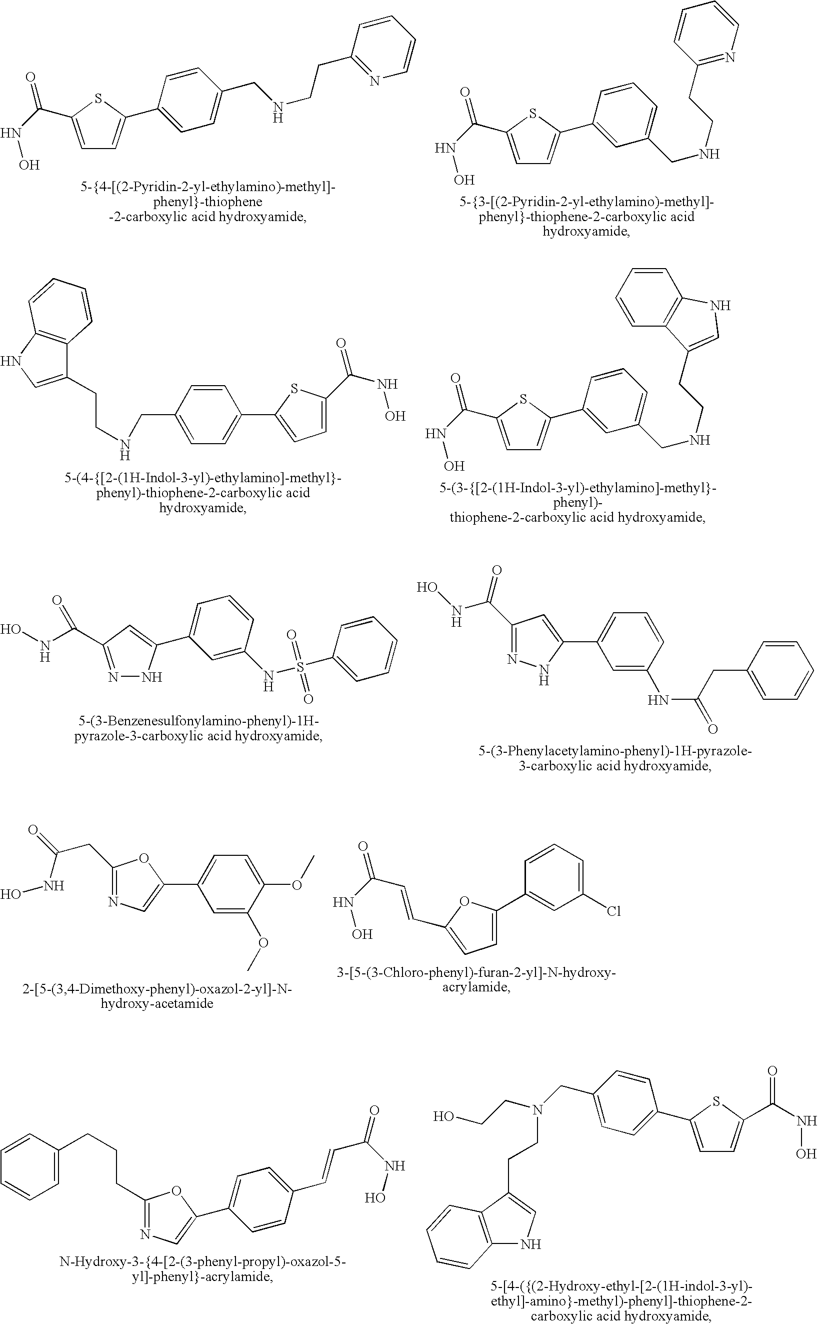

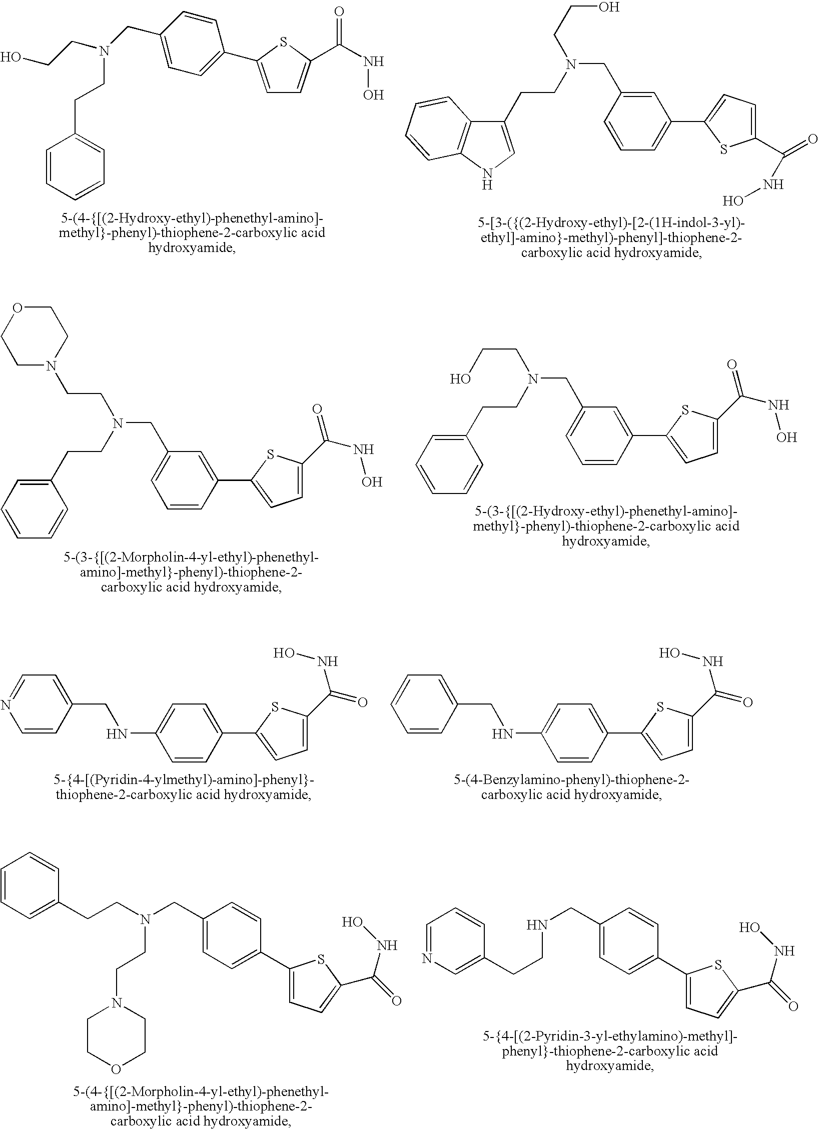

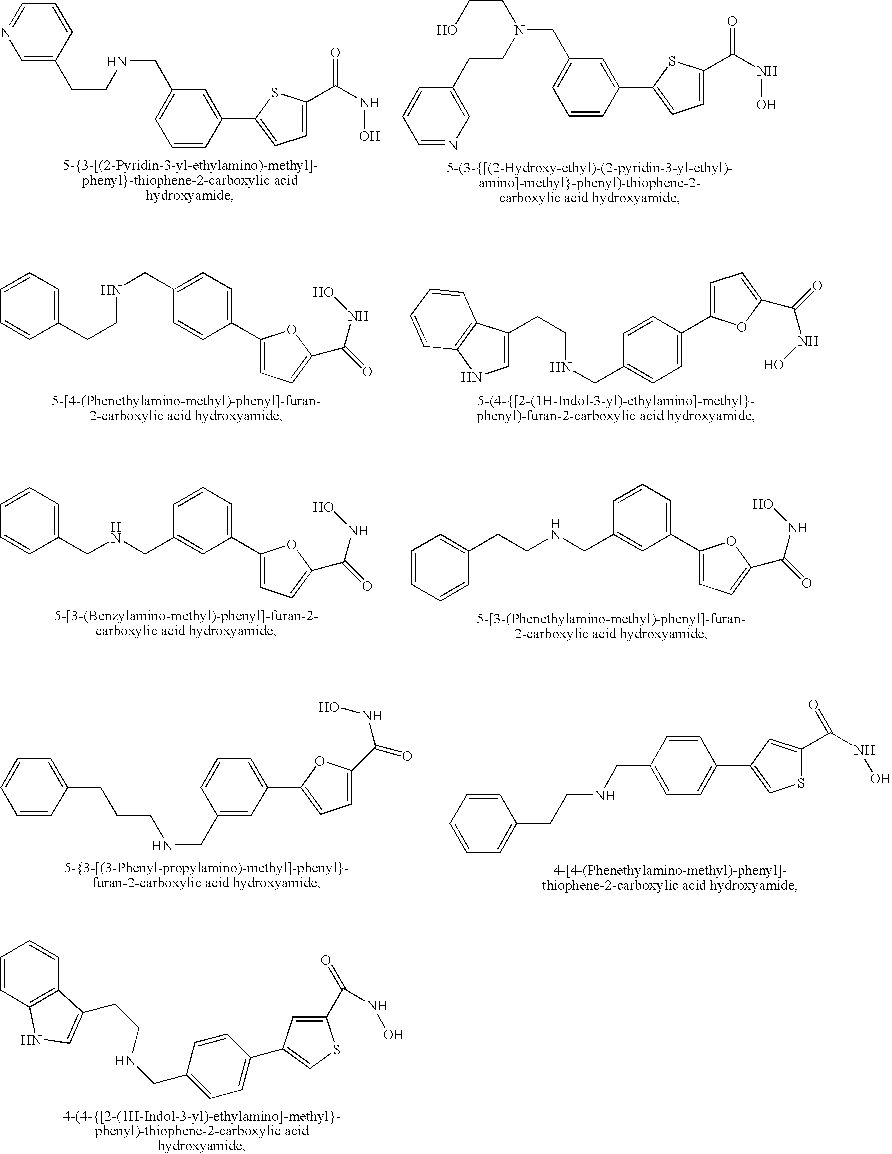

As used herein, the term unsubstituted means that there is no substituent or that the only substituents are hydrogen.

The term “optionally substituted” as used throughout the specification denotes that the group may or may not be further substituted or fused (so as to form a condensed polycyclic system), with one or more substituent groups. Preferably the substituent groups are one or more groups selected halogen, ═O, ═S, —CN, —NO₂, —CF₃, —OCF₃, alkyl, alkenyl, alkynyl, haloalkyl, haloalkenyl, haloalkynyl, heteroalkyl, cycloalkyl, cycloalkenyl, heterocycloalkyl, heterocycloalkenyl, aryl, heteroaryl, hydroxy, hydroxyalkyl, alkoxy, alkoxyalkyl, alkoxyaryl, alkoxyheteroaryl, alkenyloxy, alkynyloxy, cycloalkyloxy, cycloalkenyloxy, heterocycloalkyloxy, heterocycloalkenyloxy, aryloxy, heteroaryloxy, arylalkyl, heteroarylalkyl, cycloalkylalkyl, heterocycloalkylalkyl, arylalkyloxy, arnino, alkylamino, acylamino, aminoalkyl, arylamino, sulfonylamino, sulfinylamino, sulfonyl, alkylsulfonyl, arylsulfonyl, aminosulfonyl, aminoalkyl, alkoxyalky, CH₂heterocycloalkylCOOR₁₀ heterocycloalkylCOOR₁₀, —COOH, —COR₅, —C(O)OR₅, CONHR₅, —C(O)C(O)OR₅, C(O)CONHR₅, CON(R₅)OR₅, COCON(R₅)OR₅, NHCOR₅, CH₂NCOOR₁₀, NHCOOR₅, NHCONHR₅, C(═NOH)R₅, —SH, —SR₅, —OR₅ and acyl;
each R₅ is independently selected from the group consisting of H, alkyl, alkenyl, alkynyl, haloalkyl, heteroalkyl, cycloalkyl, heterocycloalkyl, aryl, heteroaryl, cycloalkylalkyl, heterocycloalkylalkyl, arylalkyl, heteroarylalkyl and acyl each of which may be optionally substituted;
R₁₀ is selected from H, alkyl, acyl and aryl.

“Halogen” represents chlorine, fluorine, bromine or iodine.

“Alkyl” as a group or part of a group refers to a straight or branched aliphatic hydrocarbon group, preferably a C₁-C₁₄ alkyl, more preferably C₁-C₁₀ alkyl, most preferably C₁-C₆ unless otherwise noted. Examples of suitable straight and branched C₁-C₆ alkyl substituents include methyl, ethyl, n-propyl, 2-propyl, n-butyl, sec-butyl, t-butyl, hexyl, and the like.

“Alkylamino” includes both monoalkylamino and dialkylamino, unless specified. “Monoalkylamino” means a —NH-Alkyl group, “Dialkylamino” means a —N(alkyl)₂ group, in which the alkyl is as defined as above. The alkyl group is preferably a C₁-C₆ alkyl group.

“Arylamino” includes both mono-arylamino and di-arylamino unless specified. Mono-arylamino means a group of formula aryl NH—, di-arylamino means a group of formula (aryl₂) N— where aryl is as defined herein.

“Acyl” means a group of formula G-C(═O)— or G-C(═S)— group in which the G is selected from aryl, heteroaryl, alkyl, cycloalkyl, heterocycloalkyl, arylalkyl and heteroarylalkyl as described herein. G could be further substituted. Examples of acyl include acetyl, benzoyl and phenylacetyl.

“Alkenyl” as group or part of a group denotes an aliphatic hydrocarbon group containing at least one carbon-carbon double bond and which may be straight or branched preferably having 2-14 carbon atoms, more preferably 2-12 carbon atoms, most preferably 2-6 carbon atoms, in the chain. The group may contain a plurality of double bonds in the normal chain and the orientation about each is independently E or Z. Exemplary alkenyl group include, but are not limited to, ethenyl and propenyl.

“Alkoxy” refers to an —O-alkyl group in which alkyl is defined herein. Preferably the alkoxy is a C₁-C₆alkoxy. Examples include, but are not limited to, methoxy and ethoxy.

“Alkenyloxy” refers to an —O-alkenyl group in which alkenyl is as defined herein. Preferred alkenyloxy groups are C₁-C₆ alkenyloxy groups.

“Alkynyloxy” refers to an —O-alkynyl group in which alkynyl is as defined herein. Preferred alkynyloxy groups are C₁-C₆ alkynyloxy groups.

“Alkoxycarbonyl” refers to an —C(O)—O-alkyl group in which alkyl is as defined herein. The alkyl group is preferably a C₁-C6 alkyl group. Examples include, but not limited to, methoxycarbonyl and ethoxycarbonyl.

“Akylsulfinyl” means a —S(O)-alkyl group in which alkyl is as defined above. The alkyl group is preferably a C₁-C₆ alkyl group. Exemplary alkylsulfinyl groups include, but not limited to, methylsulfinyl and ethylsulfinyl.

“Alkylsulfonyl” refers to a —S(O)₂-alkyl group in which alkyl is as defined above. The alkyl group is preferably a C₁-C₆ alkyl group. Examples include, but not limited to methylsulfonyl and ethylsulfonyl.

“Alkynyl as a group or part of a group means an aliphatic hydrocarbon group containing a carbon-carbon trip bond and which may be straight or branched preferably having from 2-14 carbon atoms, more preferably 2-12 carbon atoms in the chain, preferably 2-6 carbon atoms in the chain. Exemplary structures include, but not limited to, ethynyl and propynyl.

“Alkylaminocarbonyl” refers to an alkylamino-carbonyl group in which alkylamino is as defined above.

“Aryl” refers to a mono or fused aromatic carbocycle (ring structure having ring atoms that are all carbon) having from 5 to 12 atoms per ring. Examples of aryl groups include phenyl, naphthyl, and the like. The aryl group may be substituted by one or more substituent groups. When the aryl ring is divalent it has been referred to as “arylene” in this application.

“Arylalkenyl” means an aryl-alkenyl- group in which the aryl and alkenyl are as previously described. Exemplary arylalkenyl groups include phenylallyl.

“Arylalkyl” means an aryl-alkyl- group in which the aryl and alkyl moieties are as previously described. Preferred arylalkyl groups contains a C₁₋₅ alkyl moiety. Exemplary arylalkyl groups include benzyl, phenethyl and naphthelenemethyl.

“Cycloalkyl” refers to a saturated or partially saturated, monocyclic or fused or spiro polycyclic, carbocycle preferably containing from 3 to 9 carbons per ring, such as cyclopropyl, cyclobutyl, cyclopentyl, cyclohexyl and the like, unless otherwise specified.

The above discussion of alkyl and cycloalkyl substituents also applies to the alkyl portions of other substituents, such as without limitation, alkoxy, alkyl amines, alkyl ketones, arylalkyl, heteroarylalkyl, alkylsulfonyl and alkyl ester substituents and the like.

“Cycloalkylalkyl” means a cycloalkyl-alkyl- group in which the cycloalkyl and alkyl moieties are as previously described. Exemplary monocycloalkylalkyl groups include cyclopropylmethyl, cyclopentylmethyl, cyclohexylmethyl and cylcoheptylmethyl.

“Heterocycloalkyl” refers to an ring containing from at least one heteroatom selected from nitrogen, sulfur, oxygen, preferably from 1 to 3 heteroatoms. Each ring is preferably from 3 to 4 membered, more preferably 4 to 7 membered. Examples of suitable heterocycloalkyl substituents include pyrrolidyl, tetrahydrofuryl, tetrahydrothiofuranyl, piperidyl, piperazyl, tetrahydropyranyl, morphilino, 1,3-diazapane, 1,4-diazapane, 1,4-oxazepane, and 1,4-oxathiapane.

“Heterocycloalkenyl” refers to a heterocycloalkyl as described above but containing at least one double bond.

“Heterocycloalkylalkyl” refers to a heterocycloalkyl-alkyl group in which the heterocycloalkyl and alkyl moieties are as previously described. Exemplary heterocycloalkylalkyl groups include (2-tetrahydrofuryl)methyl, (2-tetrahydrothiofuranyl)methyl.

“Heteroalkyl” refers to a straight- or branched-chain alkyl group preferably having from 2 to 14 carbons, more preferably 2 to 10 atoms in the chain, one or more of which has been substituted by a heteroatom selected from S, O, and N. Exemplary heteroalkyls include alkyl ethers, secondary and tertiary alkyl amines, alkyl sulfides, and the like.

“Cycloalkenyl” means an optionally substituted non-aromatic monocyclic or multicyclic ring system containing at least one carbon-carbon double bond and preferably having from 5-10 carbon atoms per ring. Exemplary monocyclic cycloalkenyl rings include cyclopentenyl, cyclohexenyl or cycloheptenyl. The cycloalkenyl group may be substituted by one or more substituent groups.

“Heteroaryl” refers to a mono or fused aromatic heterocycle (ring structure preferably having a 5 to 10 member aromatic ring containing one or more heteroatoms selected from N, O and S). Typical heteroaryl substituents include furyl, thienyl, pyrrole, pyrazole, triazole, thiazole, oxazole, pyridine, pyrimidine, isoxazolyl, pyrazine, indole, benzimidazole, and the like. When the heteroaryl ring is divalent it has been referred to as “heteroaryiene” in this application.

“Heteroarylalkyl” means a heteroaryl-alkyl group in which the heteroaryl and alkyl moieties are as previously described. Preferred heteroarylalkyl groups contain a lower alkyl moiety. Exemplary heteroarylalkyl groups include pyridylmethyl.

“Lower alkyl” as a group means unless otherwise specified, an aliphatic hydrocarbon group which may be straight or branched having 1 to 6 carbon atoms in the chain, more preferably 1 to 4 carbons such as methyl, ethyl, propyl (n-propyl or isopropyl) or butyl (n-butyl, isobutyl or tertiary-butyl).

“Sulfonyl” means a G-SO₂ group in which the G is selected from aryl, heteroaryl, alkyl, cycloalkyl, heterocycloalkyl, arylalkyl and heteroarylalkyl as described herein. G could be further substituted. Examples of sulfonyl include methanesulfonyl, benzenesulfonyl, 4-methylbenzenesulfonyl, naphthalene-2-sulfonyl, and the like.

In Formula (I), as well as in Formulae la-le defining sub-sets of compounds within Formula (I), there is shown a biaryl system. In each of Formula I to 1 h, there is a requirement for attachment of an acidic moiety at one of the ring positions. This acidic moiety may be provided by, but is not limited to, groups containing a hydroxamic acid or salt derivatives of such acid which when hydrolyzed would provide the acidic moiety. In some embodiments the acidic moiety may be attached to the ring position through an alkylene group such as —CH₂— or —CH₂CH₂—, or an alkenyl group such as —CH═CH—.

It is understood that included in the family of compounds of Formula (I) are isomeric forms including diastereoisomers, enantiomers, tautomers, and geometrical isomers in “E” or “Z” configurational isomer or a mixture of E and Z isomers. It is also understood that some isomeric forms such as diastereomers, enantiomers, and geometrical isomers can be separated by physical and/or chemical methods and by those skilled in the art.

Some of the compounds of the disclosed embodiments may exist as single stereoisomers, racemates, and/or mixtures of enantiomers and/or diastereomers. All such single stereoisomers, racemates and mixtures thereof are intended to be within the scope of the subject matter described and claimed.

Additionally, Formula (I) is intended to cover, where applicable, solvated as well as unsolvated forms of the compounds. Thus, each formula includes compounds having the indicated structure, including the hydrated as well as the non-hydrated forms.

In addition to compounds of the Formula (I), the HDAC inhibiting agents of the various embodiments include pharmaceutically acceptable salts, prodrugs, and active metabolites of such compounds, and pharmaceutically acceptable salts of such metabolites.

The term “Pharmaceutically acceptable salts” refers to salts that retain the desired biological activity of the above-identified compounds, and include pharmaceutically acceptable acid addition salts and base addition salts. Suitable pharmaceutically acceptable acid addition salts of compounds of Formula (I) may be prepared from an inorganic acid or from an organic acid. Examples of such inorganic acids are hydrochloric, sulfuric, and phosphoric acid. Appropriate organic acids may be selected from aliphatic, cycloaliphatic, aromatic, heterocyclic carboxylic and sulfonic classes of organic acids, examples of which are formic, acetic, propionic, succinic, glycolic, gluconic, lactic, malic, tartaric, citric, fumaric, maleic, alkyl sulfonic, arylsulfonic. Suitable pharmaceutically acceptable base addition salts of compounds of Formula (I) include metallic salts made from lithium, sodium, potassium, magnesium, calcium, aluminium, and zinc, and organic salts made from organic bases such as choline, diethanolamine, morpholine. Other examples of organic salts are: ammonium salts, quaternary salts such as tetramethylammonium salt; amino acid addition salts such as salts with glycine and arginine. Additional information on pharmaceutically acceptable salts can be found in Remington's Pharmaceutical Sciences, 19th Edition, Mack Publishing Co., Easton, Pa. 1995. In the case of agents that are solids, it is understood by those skilled in the art that the inventive compounds, agents and salts may exist in different crystalline or polymorphic forms, all of which are intended to be within the scope of the present invention and specified formulae.

“Prodrug” means a compound which is convertible in vivo by metabolic means (e.g. by hydrolysis, reduction or oxidation) to a compound of Formula (I). For example an ester prodrug of a compound of Formula (I) containing a hydroxyl group may be convertible by hydrolysis in vivo to the parent molecule. Suitable esters of compounds of Formula (I) containing a hydroxyl group, are for example acetates, citrates, lactates, tartrates, malonates, oxalates, salicylates, propionates, succinates, fumarates, maleates, methylene-bis-β-hydroxynaphthoates, gestisates, isethionates, di-p-toluoyltartrates, methanesulphonates, ethanesulphonates, benzenesulphonates, p-toluenesulphonates, cyclohexylsulphamates and quinates. As another example an ester prodrug of a compound of Formula (I) containing a carboxy group may be convertible by hydrolysis in vivo to the parent molecule. (Examples of ester prodrugs are those described by F. J. Leinweber, Drug Metab. Res.,18:379, 1987).

Possible HDAC inhibiting agents include those having an IC50 value of 5 μM or less.

Administration of compounds within Formula (I) to humans can be by any of the accepted modes for enteral administration such as oral or rectal, or by parenteral administration such as subcutaneous, intramuscular, intravenous and intradermal routes. Injection can be bolus or via constant or intermittent infusion. The active compound is typically included in a pharmaceutically acceptable carrier or diluent and in an amount sufficient to deliver to the patient a therapeutically effective dose. In various embodiments the inhibitor compound may be selectively toxic or more toxic to rapidly proliferating cells, e.g. cancerous tumors, than to normal cells.

The term “therapeutically effective amount” or “effective amount” is an amount sufficient to effect beneficial or desired clinical results. An effective amount can be administered in one or more administrations. An effective amount is typically sufficient to palliate, ameliorate, stabilize, reverse, slow or delay the progression of the disease state. A therapeutically effective amount can be readily determined by a skilled practitioner by the use of conventional techniques and by observing results obtained in analogous circumstances. In determining the effective amount a number of factors are considered including the species of the patient, its size, age, general health, the specific disease involved, the degree or severity of the disease, the response of the individual patient, the particular compound administered, the mode of administration, the bioavailability of the compound, the dose regimen selected, the use of other medication and other relevant circumstances.

In using the compounds of the invention they can be administered in any form or mode which makes the compound bioavailable. One skilled in the art of preparing formulations can readily select the proper form and mode of administration depending upon the particular characteristics of the compound selected, the condition to be treated, the stage of the condition to be treated and other relevant circumstances. We refer the reader to Remingtons Pharmaceutical Sciences, 19^{th} edition, Mack Publishing Co. (1995) for further information.

The compounds of the present invention can be administered alone or in the form of a pharmaceutical composition in combination with a pharmaceutically acceptable carrier, diluent or excipient. The compounds of the invention, while effective themselves, are typically formulated and administered in the form of their pharmaceutically acceptable salts as these forms are typically more stable, more easily crystallised and have increased solubility.

The compounds are, however, typically used in the form of pharmaceutical compositions which are formulated depending on the desired mode of administration. As such in a further embodiment the present invention provides a pharmaceutical composition including a compound of Formula (I) and a pharmaceutically acceptable carrier, diluent or excipient. The compositions are prepared in manners well known in the art.

The invention in other embodiments provides a pharmaceutical pack or kit comprising one or more containers filled with one or more of the ingredients of the pharmaceutical compositions of the invention. In such a pack or kit can be found a container having a unit dosage of the agent (s). The kits can include a composition comprising an effective agent either as concentrates (including lyophilized compositions), which can be diluted further prior to use or they can be provided at the concentration of use, where the vials may include one or more dosages. Conveniently, in the kits, single dosages can be provided in sterile vials so that the physician can employ the vials directly, where the vials will have the desired amount and concentration of agent(s). Associated with such container(s) can be various written materials such as instructions for use, or a notice in the form prescribed by a governmental agency regulating the manufacture, use or sale of pharmaceuticals or biological products, which notice reflects approval by the agency of manufacture, use or sale for human administration.

The compounds of the invention may be used or administered in combination with one or more additional drug (s) that include chemotherapeutic drugs or HDAC inhibitor drugs and/or procedures (e.g. surgery, radiotherapy) for the treatment of the disorder/diseases mentioned. The components can be administered in the same formulation or in separate formulations. If administered in separate formulations the compounds of the invention may be administered sequentially or simultaneously with the other drug (s).

In addition to being able to be administered in combination with one or more additional drugs that include chemotherapeutic drugs or HDAC inhibitor drugs the compounds of the invention may be used in a combination therapy. When this is done the compounds are typically administered in combination with each other. Thus one or more of the compounds of the invention may be administered either simultaneously (as a combined preparation) or sequentially in order to achieve a desired effect. This is especially desirable where the therapeutic profile of each compound is different such that the combined effect of the two drugs provides an improved therapeutic result.

Pharmaceutical compositions of this invention for parenteral injection comprise pharmaceutically acceptable sterile aqueous or nonaqueous solutions, dispersions, suspensions or emulsions as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol, and the like), and suitable mixtures thereof, vegetable oils (such as olive oil), and injectable organic esters such as ethyl oleate. Proper fluidity can be maintained, for example, by the use of coating materials such as lecithin, by the maintenance of the required particle size in the case of dispersions, and by the use of surfactants.

These compositions may also contain adjuvants such as preservative, wetting agents, emulsifying agents, and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents such as sugars, sodium chloride, and the like. Prolonged absorption of the injectable pharmaceutical form may be brought about by the inclusion of agents that delay absorption such as aluminium monostearate and gelatin.

If desired, and for more effective distribution, the compounds can be incorporated into slow release or targeted delivery systems such as polymer matrices, liposomes, and microspheres.

The injectable formulations can be sterilized, for example, by filtration through a bacterial-retaining filter, or by incorporating sterilizing agents in the form of sterile solid compositions that can be dissolved or dispersed in sterile water or other sterile injectable medium just prior to use.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like.

The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes.

If desired, and for more effective distribution, the compounds can be incorporated into slow release or targeted delivery systems such as polymer matrices, liposomes, and microspheres.

The active compounds can also be In microencapsulated form, if appropriate, with one or more of the above-mentioned excipients.

Liquid dosage forms for oral administration include pharmaceutically acceptable emulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethyl formamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, and perfuming agents.

Suspensions, in addition to the active compounds, may contain suspending agents as, for example, ethoxylated isostearyl alcohols, polyoxyethylene sorbitol and sorbitan esters, microcrystalline cellulose, aluminium metahydroxide, bentonite, agar-agar, and tragacanth, and mixtures thereof.

Compositions for rectal or vaginal administration are preferably suppositories which can be prepared by mixing the compounds of this invention with suitable non-irritating excipients or carriers such as cocoa butter, polyethylene glycol or a suppository wax which are solid at room temperature but liquid at body temperature and therefore melt in the rectum or vaginal cavity and release the active compound.

Dosage forms for topical administration of a compound of this invention include powders, patches, sprays, ointments and inhalants. The active compound is mixed under sterile conditions with a pharmaceutically acceptable carrier and any needed preservatives, buffers, or propellants which may be required.

A preferred dosage will be a range from about 0.01 to 300 mg per kilogram of body weight per day. A more preferred dosage will be in the range from 0.1 to 100 mg per kilogram of body weight per day, more preferably from 0.2 to 80 mg per kilogram of body weight per day, even more preferably 0.2 to 50 mg per kilogram of body weight per day. A suitable dose can be administered in multiple sub-doses per day.

As discussed above, the compounds of the embodiments disclosed inhibit histone deacetylases. The enzymatic activity of a histone deacetylase can be measured using known methodologies [Yoshida M. et al, J. Biol. Chem., 265, 17174 (1990), J. Taunton et al, Science 1996 272: 408]. In certain embodiments, the histone deacetylase inhibitor interacts with and/or reduces the activity of more than one histone deacetylase in the cell, which can either be from the same class of histone deacetylase or different class of histone deacetylase. In some other embodiments, the histone deacetylase inhibitor interacts and/or reduces the activity of predominantly one histone deacetylase, for example HDAC-1, HDAC-3 or HDAC-8 which belongs to Class I HDAC enzymes [De Ruijter A.J.M. et al, Biochem. J., 370, 737-749 (2003)]. Certain preferred histone deacetylase inhibitors are those that interact with, and/or reduce the activity of a histone deacetylase which is involved in tumorigenesis, and these compounds may be useful for treating proliferative diseases. Examples of such cell proliferative diseases or conditions include cancer (include any metastases), psoriasis, and smooth muscle cell proliferative disorders such as restenosis. The inventive compounds may be particularly useful for treating tumors such as breast cancer, lung cancer, ovarian cancer, prostate cancer, head and/or neck cancer, or renal, gastric, colon cancer, pancreatic cancer and brain cancer as well as hematologic malignancies such as lymphomas and leukemias. In addition, the inventive compounds may be useful for treating a proliferative disease that is refractory to the treatment with other chemotherapeutics; and for treating hyperproliferative condition such as leukemias, psoriasis and restenosis. In other embodiments, compounds in this invention can be used to treat pre-cancer conditions including myeloid dysplasia, endometrial dysplasia and cervical dysplasia.

Additionally compounds of the various embodiments disclosed herein may be useful for treating neurodegenerative diseases, and inflammatory diseases and/or immune system disorders.

The disorder is preferably selected from the group consisting of cancer, inflammatory diseases and/or immune system disorders (e.g. rheumatoid arthritis, systemic lupus erythematosus), angioflbroma, cardiovascular diseases, fibrotic diseases, diabetes, autoimmune diseases, chronic and acute neurodegenerative disease like Huntington's disease, Parkinson's disease, disruptions of nerval tissue and infectious diseases like fungal, bacterial and viral infections. In another embodiment the disorder is a proliferative disorder.

The histone deacetylase inhibitors of the invention have significant antiproliferative effects and promote differentiation, cell cycle arrest in the G1 or G2 phase, and induce apoptosis.

### Synthesis of Deacetylase Inhibitors

The agents of the various embodiments may be prepared using the reaction routes and synthesis schemes as described below, employing the techniques available in the art using starting materials that are readily available. The preparation of particular embodiments is described in detail in the following examples, but the artisan will recognize that the chemical reactions described may be readily adapted to prepare a number of other agents of the various embodiments. For example, the synthesis of non-exemplified compounds may be successfully performed by modifications apparent to those skilled in the art, e.g., by appropriately protecting interfering groups, by changing to other suitable reagents known in the art, or by making routine modifications of reaction conditions. A list of suitable protecting groups in organic synthesis can be found in T. W. Greene and P. G. M. Wuts' Protective Groups in Organic Synthesis, 3^{rd} Edition, Wiley InterScience, 1999. Alternatively, other reactions disclosed herein or known in the art will be recognized as having applicability for preparing other compounds of the various embodiments.

Reagents useful for synthesizing compounds may be obtained or prepared according to techniques known in the art.

In the examples described below, unless otherwise indicated, all temperatures in the following description are in degrees Celsius and all parts and percentages are by weight, unless indicated otherwise.

Various starting materials and other reagents were purchased from commercial suppliers, such as Aldrich Chemical Company or Lancaster Synthesis Ltd., and used without further purification, unless otherwise indicated. Tetrahydrofuran (THF) and N,N-dimethylformamide (DMF) were purchased from Aldrich in SureSeal bottles and used as received. All solvents were purified by using standard methods in the art, unless otherwise indicated.

The reactions set forth below were performed under a positive pressure of nitrogen, argon or with a drying tube, at ambient temperature (unless otherwise stated), in anhydrous solvents, and the reaction flasks are fitted with rubber septa for the introduction of substrates and reagents via syringe. Glassware was oven-dried and/or heat-dried. Analytical thin-layer chromatography was performed on glass-backed silica gel 60 F254 plates (E Merck (0.25 mm)) and eluted with the appropriate solvent ratios (v/v). The reactions were assayed by TLC and terminated as judged by the consumption of starting material.

The TLC plates were visualized by UV absorption or with a p-anisaldehyde spray reagent or a phosphomolybdic acid reagent (Aldrich Chemical, 20 wt % in ethanol) which was activated with heat, or by staining in iodine chamber. Work-ups were typically done by doubling the reaction volume with the reaction solvent or extraction solvent and then washing with the indicated aqueous solutions using 25% by volume of the extraction volume (unless otherwise indicated). Product solutions were dried over anhydrous sodium sulfate prior to filtration, and evaporation of the solvents was under reduced pressure on a rotary evaporator and noted as solvents removed in vacuo. Flash column chromatography [Still et al, J. Org. Chem., 43, 2923 (1978)] was conducted using E Merck-grade flash silica gel (47-61 mm) and a silica gel:crude material ratio of about 20:1 to 50:1, unless otherwise stated. Hydrogenolysiss was done at the pressure indicated or at ambient pressure.

“Workup” means the reaction mixture or the residue of a reaction mixture obtained by removing the organic solvent, was extracted with a suitable organic solvent such as EtOAC or CH₂Cl₂ and the organic layer was washed with water, or a dilute base (aqueous sodium bicarbonate or carbonate) or acid (aqueous hydrochloric acid) when necessary, brine; and the organic layer was dried over anhydrous Na₂SO₄ or MgSO₄, filtered; and the filtrate was evaporated to dryness under reduced pressure to remove organic solvent. The residue will provide a product or will be used for further purification.

Reverse-phase preparative HPLC (RPHPLC) was operated by using a C₁₈ column (5 um, 21.2×150 mm) at flow rate of 20 mL/min and a linear gradient from 5 to 95% of CH₃CN+0.1% TFA over 18 min. High-throughput mass-dependent (reverse-phase HPLC) purification system (HTP) was operated by using a C₁₈ column (5 um, 19×50 mm) at flow rate of 30 mL/min and a linear gradient from 5 to 95% of CH₃CN+0.05% TFA over 9 min. The fractions containing the desire product were lyophilized, or evaporated to dryness under vacuum to provide the dry compound, or evaporated to remove the volatile organic solvent then extracted with organic solvents (ethyl acetate or dichloromethane are commonly used, if necessary, the pH of the aqueous solution could also be adjusted in order to get free base, acid or the neutral compound).

¹H NMR spectra were recorded on a Bruker instrument operating at 400 MHz, and ¹³C-NMR spectra were recorded operating at 100 MHz. NMR spectra are obtained as CDCl₃ solutions (reported in ppm), using chloroform as the reference standard (7.26 ppm and 77.0 ppm) or CD₃OD (3.3 and 4.8 ppm and 49.3 ppm) or CD₃SOCD₃ (2.50 and 39.5 ppm), or an internal tetramethylsilane standard (0.00 ppm) when appropriate. Other NMR solvents were used as needed. When peak multiplicities are reported, the following abbreviations are used: s=singlet, d=doublet, t=triplet, m=multiplet, br=broadened, dd=doublet of doublets, dt=doublet of triplets. Coupling constants, when given, are reported in Hertz.

Mass spectra were obtained using LC/MS either in ESI or APCI. All melting points are uncorrected.

All final products had greater than 90% purity (by HPLC at wavelengths of 220 nm and 254 nm).

The following examples are intended to illustrate the embodiments disclosed and are not to be construed as being limitations thereto. Additional compounds, other than those described below, may be prepared using the following described reaction scheme or appropriate variations or modifications thereof.

### Synthesis

Scheme I illustrates the procedure used for preparing compounds of Formula (I), wherein B is a thiazole ring. Compounds of Formula (I) can be prepared by analogous procedure, for example, by the choice of appropriate starting material. For example, in the case where A is thiophene and B is thiazole in Formula (I), such compound(s) can be synthesized by analogous method illustrated in Scheme I starting with [5-(2-Chloro-acetyl)-thiophen-2-yl]-acetic acid, thiourea, and appropriate acyl chloride component, anhydride component, sulfonyl chloride component or aldehyde component, and appropriate hydroxylamine or N-alkyl hydroxylamine (NHR₁OH where R₁ is defined as above).

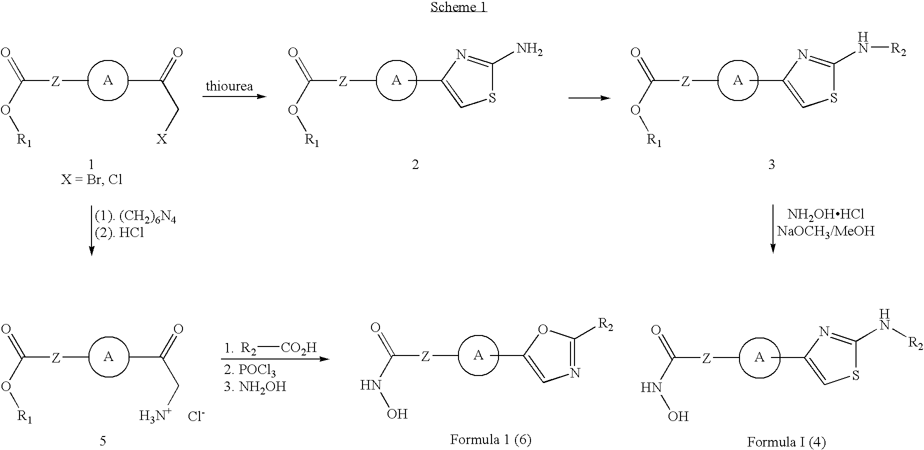

Specifically, the hydroxamate compounds Examples 1-12, 13-16 and 17 of the present invention can be synthesized by the synthetic route shown in Scheme 1. The synthesis of the hydroxamate compounds started with ester (1) that was either commercially available or obtained through treatment of appropriate carboxylic acid in methanol under acid catalysis (e.g., hydrogen chloride, hydrochloric acid, sulphuric acid). The coupling reaction of (1) with thiourea in appropriate solvent (e.g. methanol or ethanol) gave 2-amino-thiazole methyl ester (2). Treatment of (2) with various acyl chloride, anhydride, sulfonyl chloride or aldehyde under appropriate reaction conditions resulted substituted thiophene-thiazole methyl esters (3). The hydroxamate compounds were obtained by a known synthesis method (J. Med. Chem., 2002, 45, 753-757).

Compound 1 was also converted to amino ketone (5) by reacting it with hexamethylenetetramine and then hydrolysis. The amino ketone 5 was coupled with a carboxylic acid or acid chloride and the resultant amide was dehydrated with POCl₃ or the like to give an oxazole ring. The ester was further converted to hydroxamates (6).

The following preparation and examples are given to enable those skilled in the art to more clearly understand and to practice the present invention. They should not be considered as limiting the scope of the invention, but merely as being illustrative and representative thereof.

### EXAMPLE 1

### Preparation of N-Hydroxy-2-[5-(2-phenylacetylamino-thiazol-4-yl)-thiophen-2-yl]-acetamide

Step 1

Synthesis of [5-(2-Chloro-acetyl)-thiophene-2-yl]-acetic acid methyl ester

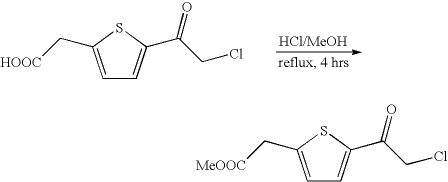

To a solution of 463 mg [5-(2-Chloro-acetyl)-thiophene-2-yl]-acetic acid in 4 mL MeOH was added 1 mL 37% HCl at room temperature. The reaction was heated to reflux for 4 hours. The reaction was cooled to room temperature, neutralized by saturated aqueous sodium bicarbonate and extracted by dichloromethane. The combined organic layer was washed with brine, dried over anhydrous sodium sulfate and concentrated in vacuo. The crude product was purified by flash chromatography on silica gel to afford the desired product 411 mg (84%). Rf 0.6 (hexane:ethyl acetate=1:1); ¹H NMR (CDCl₃): δ 7.67 (d, J=3.9 Hz, 1H), 7.03 (d, J=3.9 Hz, 1H), 4.55 (s, 2H), 3.89 (s, 2H), 3.76 (s, 3H); ¹³C NMR (CDCl₃): δ 184.1, 169.7, 145.8, 140.3, 133.3, 128.5, 52.7, 45.4, 35.9; ESIMS (m/z) 233 (M+1)

Step 2

Synthesis of [5-(2-Amino-thiazol-4-yl)-thiophen-2-yl]-acetic acid methyl ester

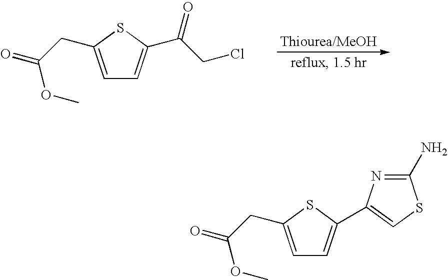

To a solution of [5-(2-Chloro-acetyl)-thiophene-2-yl]-acetic acid methyl ester (56.5 mg) in MeOH (1 mL) was added thiourea (22 mg) at room temperature. The reaction was heated to reflux for 1.5 hour. The reaction was cooled to room temperature and methanol was removed in vacuo to afford the desired product (56 mg (92%). The product was used directly for further reaction without purification. Rf 0.5 (hexane:ethyl acetate=1:1); ESIMS (m/z) 255 (M+1). ¹H NMR (DMSO-d₆): δ 7.33 (d, J=3.6 Hz, 1H), 6.93 (d, J=3.7 Hz, 1H), 6.89 (s, 1H), 3.94 (s, 2H), 3.64 (s, 3H); ¹³C NMR (DMSO-d₆): δ 170.5, 168.7, 141.5, 136.3, 134.9, 127.7, 123.3, 100.0, 51.9, 34.6.

Step 3

Synthesis of [5-(2-Phenylacetylamino-thiazol-4-yl)-thiophen-2-yl]-acetic acid methyl ester

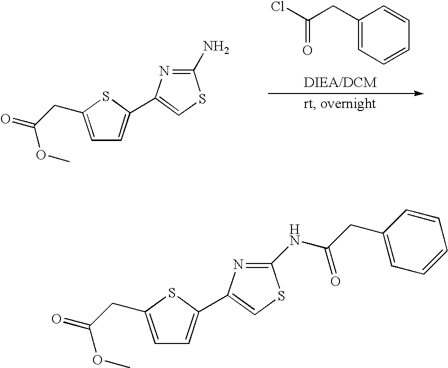

To a solution of [5-(2-Amino-thiazol-4-yl)-thiophen-2-yl]-acetic acid methyl ester (136 mg) in DCM (2 mL) was added phenylacetyl chloride (80 μL) and diisopropylethyl amine (170 μL) at room temperature. The reaction was stirred at room temperature for overnight. The reaction was quenched by water, extracted by DCM, washed by 1M HCl, saturated aqueous sodium bicarbonated and brine. The combined organic layer was dried over anhydrous sodium sulfate and concentrated in vacuo to afford the crude product (130 mg, 70%). The crude product was used directly for further reaction without purification; ESIMS (m/z) 373 (M+1)

Step 4

Synthesis of N-Hydroxy-2-[5-(2-phenylacetylamino-thiazol-4-yl)-thiophen-2-yl]-acetamide

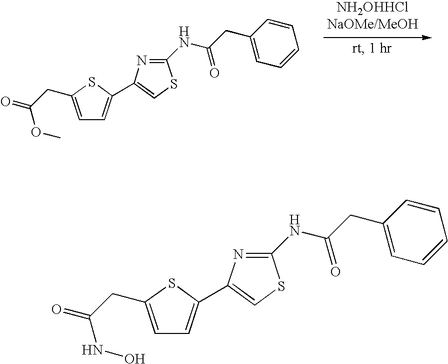

To a mixture of [5-(2-Phenylacetylamino-thiazol-4-yl)-thiophen-2-yl]-acetic acid methyl ester (50 mg) in MeOH (0.5 mL) was added hydroxylamine hydrochloride (13 mg) and NaOMe (30% in methanol, 74 μL) at room temperature. The reaction was stirred at room temperature for 1 hour. 1N HCl was added dropwise to the reaction until clear solution obtained. The desired product was obtained through reverse phase prep-HPLC (21 mg, 42%). ESIMS (m/z) 374 (M+1); ¹H NMR (CD₃OD): δ 7.29-7.22 (m, 5H), 7.21 (d, J=3.6 Hz, 1H), 7.09 (s, 1H), 6.83 (d, J=3.6 Hz), 3.73 (s, 2H), 3.54 (s, 2H); ¹³C NMR (CD₃OD): δ 169.8, 167.6, 157.4, 143.9, 137.4, 135.4, 133.7, 128.3, 127.8, 126.4, 126.3, 122.5, 105.2, 41.3, 33.1.

### EXAMPLE 2

### Preparation of 2-[5-(2-Amino-thiazol-4-yl)-thioiphen-2-yl]-N-hydroxy-acetamide

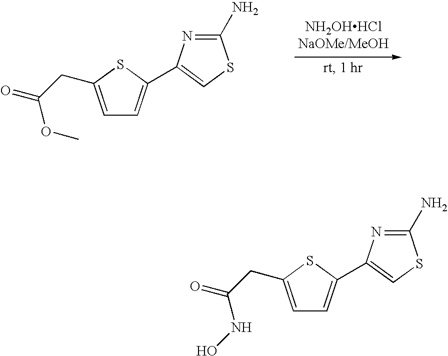

Proceeding as described in Example 1 above but using appropriate starting materials, the titled compound was prepared. ¹H NMR (DMSO-d₆): δ 7.20 (s, 1H), 6.84 (s, 1H), 6.80 (d, J=2.6 Hz, 1H), 3.49 (s, 2H); ESIMS (m/z) 256 (M+1)

### EXAMPLE 3

### Pretaration of 2-[5-(2-Benzylamino-thiazol-4-yl)-thiophen-2-yl]-N-hydroxy-acetamide

Step 1

Synthesis of [5-(2-Benzylamino-thiazol-4-yl)-thiophen-2-yl]-acetic acid methyl ester

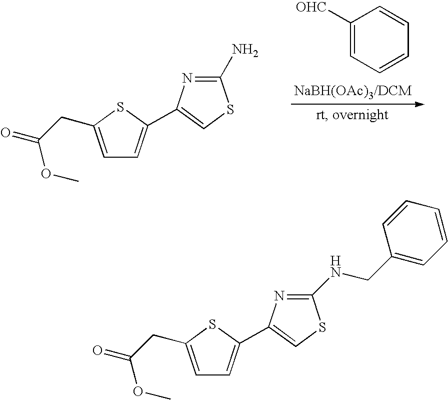

A mixture of [5-(2-Amino-thiazol-4-yl)-thiophen-2-yl]-acetic acid methyl ester (76.2 mg, refer to Example 1) in DCM (1 mL) was treated by NaBH(OAc)₃ at room temperature. The reaction was stirred at room temperature for overnight. The reaction was quenched by cold water and purified by reverse phase prep-HPLC to afford the desired product (6.9 mg, 7%). ¹H NMR (CDCl₃): δ 7.50 (d, J=3.7 Hz, 1H), 7.45-7.43 (m, 5H), 6.98 (d, J=3.7 Hz, 1H), 6.43 (s, 1H), 4.57 (s, 2H), 3.90 (s, 2H), 3.81 (s, 3H); 13C NMR (CDCl₃): δ 170.8, 170.5, 138.5, 136.6, 134.9, 129.1, 128.5, 128.4, 127.9, 127,8, 126.6, 97.9, 52.6, 50.6, 35.5; ESIMS (m/z) 345 (M+1)

Step 2

2-[5-(2-Benzylamino-thiazol-4-yl)-thiophen-2-yl]-N-hydroxy-acetamide

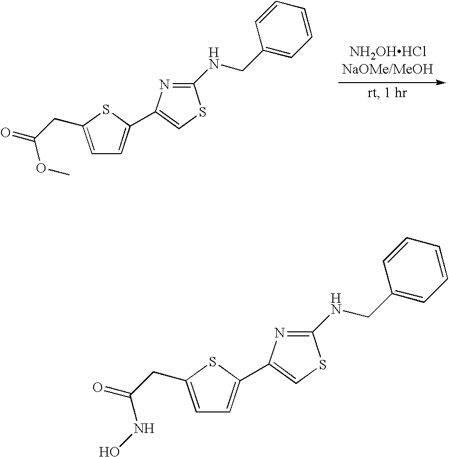

Proceeding as described in Example 1 above but using appropriate starting materials, the titled compound was prepared. ¹H NMR (DMSO-d₆): δ 10.67 (s, 1H), 8.24 (s, 1H), 7.40-5 7.18 (m, 8H), 6.81 (s, 2H), 4.46 (s, 2H); ¹³C NMR (DMSO-d₆): δ 168.3, 165.9, 144.5, 138.9, 136.3, 128.3, 127.6, 167.0, 126.6, 122.4, 99.5, 99.0, 47.8, 33.9; ESIMS (m/z) 346 (M+1)

### EXAMPLE 4

### Preparation of 2-[5-(2-Acetylamino-thiazol-4-yl)-thiorhen-2-yl]-N-hydroxy-acetamide

Step 1

Synthesis of [5-(2-Acetylamino-thiazol-4-yl)-thiophen-2-yl]-acetic acid methyl ester

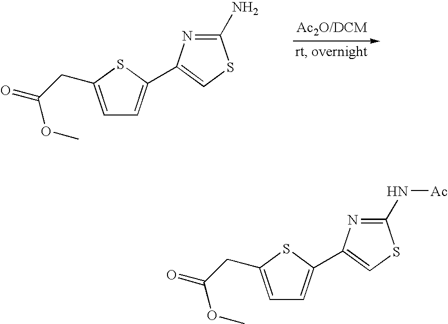

A mixture of [5-(2-Amino-thiazol-4-yl)-thiophen-2-yl]-acetic acid methyl ester (52 mg) in DCM (0.5 mL) was treated by acetic anhydride (94 μL) at room temperature. The reaction was stirred at room temperature for overnight. The reaction was quenched by aqueous sodium bicarbonate, extracted by DCM. The combined organic layer was dried over anhydrous sodium sulphate and concentrated in vacuo to afford the crude product 42 mg which was used directly for further reaction. ESIMS (m/z) 297 (M+1)

Step 2

Synthesis of 2-[5-(2-Acetylamino-thiazol-4-yl)-thiophen-2-yl]-N-hydroxy-acetamide

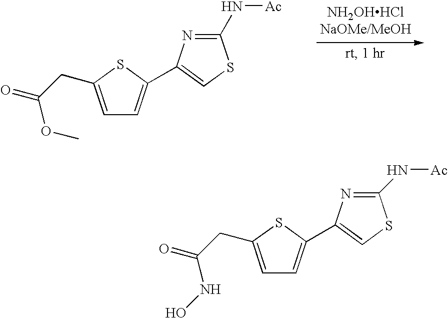

Proceeding as described in Example 1 above but using appropriate starting materials, the titled compound was prepared. ¹H NMR (DMSO-d₆): δ 12.26 (s, 1H), 10.68 (s, 1H), 7.35 (s, 1H), 7.30 (d, J=3.6 Hz, 2H), 6.86 (d, J=3.6 Hz, 2H), 3.52 (s, 2H), 2,15 (s, 3H); ESIMS (m/z) 298 (M+1)

### EXAMPLE 5

### Preparation of {5-[4-(5-Hydroxycarbamoylmethyl-thiophen-2-yl)-thiazol-2-ylcarbamoyl]-benzofuran-2-ylmethyl}carbamic acid tert-butyl ester

Step 1

Synthesis of [5-(2-{[2-(tert-Butoxycarbonylamino-methyl)-benzofuran-5-carbonyl]-amino}-thiazol-4-yl)-thiophen-2-yl]-acetic acid methyl ester

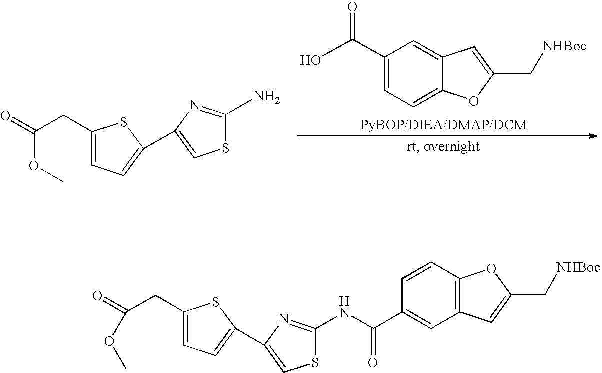

A mixture of [5-(2-Amino-thiazol4-yl)-thiophen-2-yl]-acetic acid methyl ester (20 mg) and (Benzotriazol-1-yloxy)tripyrrolidinophosphonium Hexafluorophosphate (PyBOP, 70 mg) and 2-(tert-Butoxycarbonylamino-methyl)-benzofuran-5-carboxylic acid (31 mg) in DCM (1 mL) was treated by DMAP (2 mg) and DIEA (50 μL) at room temperature. The solution was stirred at room temperature for overnight. The reaction was subjected to reverse phase prep-HPLC for purification. A total of 11 mg (26%) desired product was obtained. Rf 0.76 (hexane:ethyl acetate=1:1); ¹H NMR (DMSO-d₆): δ 8.35 (d, J=1.7 Hz, 1H), 8.03 (dd, J=1.9 Hz, 8.7 Hz, 1H), 7.58 (d, J=8.7 Hz, 1H), 7.44 (d, J=3.7 Hz, 1H), 7.01 (s, 1H), 6.96 (d, J=3.7. Hz, 1H), 6.74 (s, 1H), 5.03 (br, 1H), 4.51 (d, J=5.3 Hz, 2H), 3.88 (s, 2H), 3.78 (s, 3H), 1.50 (s, 9H)

Step 2

Synthesis of {5-[4-(5-Hydroxycarbamoylmethyl-thiophen-2-yl)-thiazol-2-ylcarbamoyl]-benzofuran-2-ylmethyl}carbamic acid tert-butyl ester

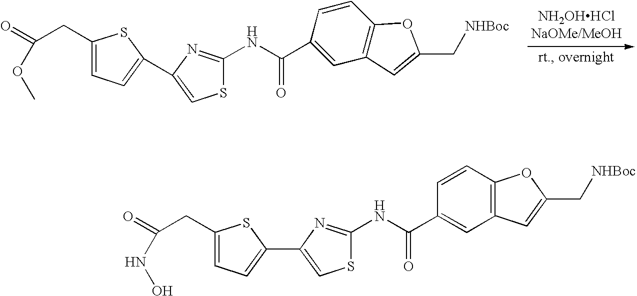

Proceeding as described in Example 1 above but using appropriate starting materials, the titled compound was prepared. ESIMS (m/z) 529 (M+1)

The following compounds are prepared by methods analogous to those disclosed in Examples 5

TABLE 1
Representative examples made by method analogous to Example 5

| Example | Structures | m/z [MH]⁺ |
|---|---|---|
| 6 | 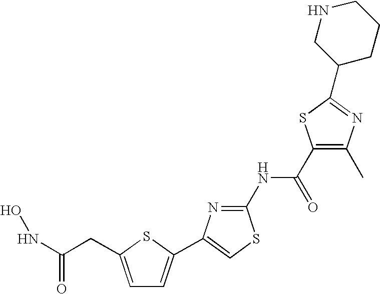 | 463 |
| 7 | 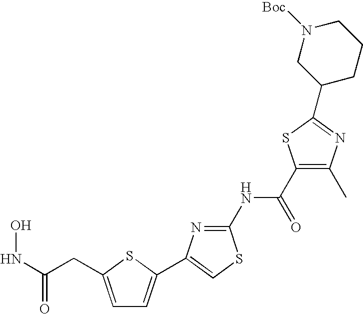 | 564 |
| 8 | 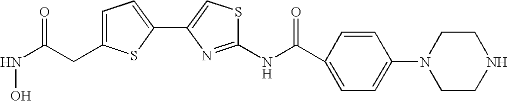 | 444 |
| 9 | 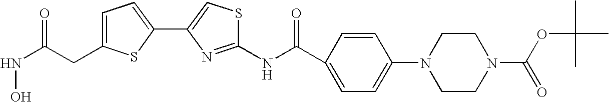 | 544 |
| 10 | 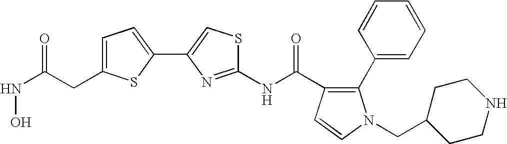 | 522 |
| 11 | 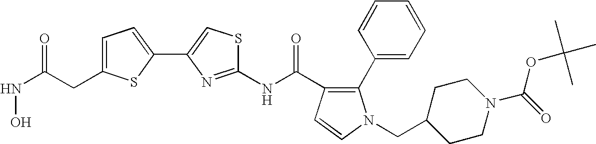 | 622 |
| 12 | 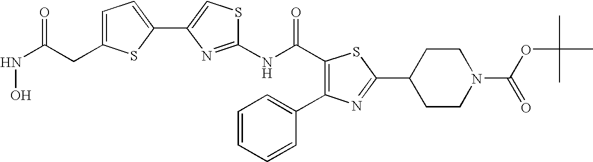 | 626 |

### EXAMPLE 13

### Preparation of 5-(2-Benzoylamino-thiazol-4-yl)-thiophene-2-carboxylic acid hydroxyamide

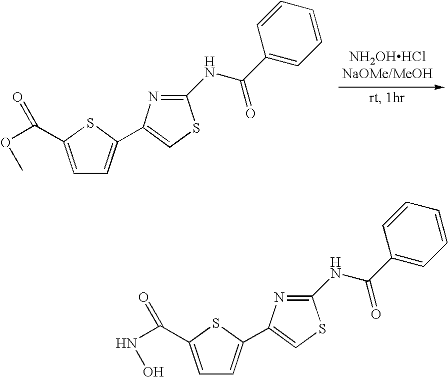

Proceeding as described in Example 1 above but using appropriate starting materials, the titled compound was prepared. ¹H NMR (DMSO-d₆): δ 12.84 (s, 1H), 11.23 (br, IH), 8.13-8.11 (m, 2H), 7.69-7.54 (m, 6H); ¹³C NMR (DMSO-d₆): δ 171.3, 165.4, 158.9, 143.3, 141.7, 135.7, 132.7, 131.8, 128.6, 128.2, 124.2, 109.1; ESIMS (m/z) 346 (M+1)

### EXAMPLE 14

### Preparation of 5-(2-Phenylacetylamino-thiazol-4-yl)-thiophene-2-carboxylic acid hydroxyamide

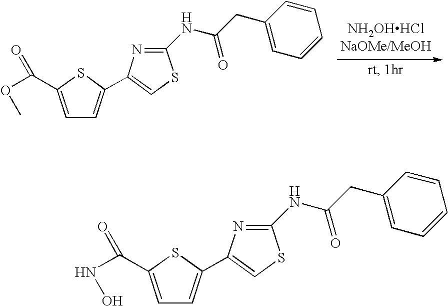

Proceeding as described in Example 1 above but using appropriate starting materials, the titled compound was prepared. ¹H NMR (DMSO-d₆): δ 12.60 (s, 1H), 11.22 (br, 1H), 7.60-7.49 (m, 3H), 7.35-7.26 (m, 5H), 3.79 (s, 2H); ¹³C NMR (DMSO-d₆): δ 169.6, 159.6, 158.2, 143.0, 141.8, 135.8, 134.8, 129.2, 128.4, 126.8, 124.1, 108.5, 41.6; ESIMS (m/z) 360 (M+1)

### EXAMPLE 15

### Preparation of 5-(2-Benzenesulfonylamino-thiazol-4-yl)-thiophene-2-carboxylic acid hydroxyamide

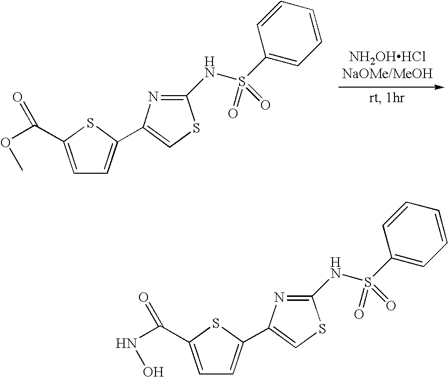

Proceeding as described in Example 1 above but using appropriate starting materials, the titled compound was prepared. ¹H NMR (DMSO-d₆): δ 11.34 (s, 1H), 9.24 (br, 1H), 7.87-7.85 (m, 2H), 7.63-7.55 (m, 4H, Ar—H), 7.45 (d, J=3.9 Hz), 7.21 (s, 1H); ¹³C NMR (DMSO-d₆): δ 167.4, 158.8, 141.6, 137.5, 135.2, 132.4, 129.1, 127.8, 126.1, 125.9, 105.0; ESIMS (m/z) 382 (M+1)

### EXAMPLE 16

### Preparation of 5-[2-(2-Phenoxy-acetamino)-thiazol-4-yl]-thiophene-2-carboxylic acid hydroxyamide

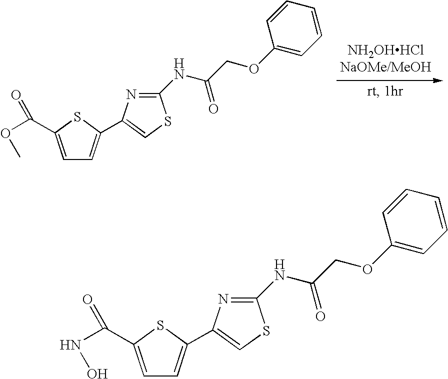

Proceeding as described in Example 1 above but using appropriate starting materials, the titled compound was prepared. ¹H NMR (DMSO-d₆): o 12.63 (s, 1H), 11.24 (s, 1H), 7.65-7.51 (m, 3H), 7.34-7.30 (m, 2H), 7.00-6.96 (m, 3H), 4.88 (s, 2H); ¹³C NMR (DMSO-d₆):δ 167.2, 159.5, 157.7, 143.1, 141.7, 135.9, 129.5, 128.4, 124.2, 121.2, 114.5, 108.8, 65.9; ESIMS (m/z) 376 (M+1)

### EXAMPLE 17

### Preparation of 5-(2-Phenylacetylamino-thiazol-4-yl)-isoxazole-3-carboxylic acid hydroxyamide

Step 1

Synthesis of 5-(2-Amino-thiazol-4-yl)-isoxazole-3-carboxylic acid ethyl ester

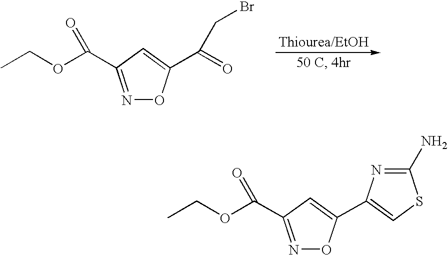

Proceeding as described in Example 1 above but using appropriate starting materials, the titled compound was prepared. Rf 0.7 (Hexane:Ethyl Acetate=1:1) ESIMS (m/z) 240 (M+1)

Step 2

Synthesis of 5-(2-Phenylacetylamino-thiazol-4-yl)-isoxazole-3-carboxylic acid ethyl ester

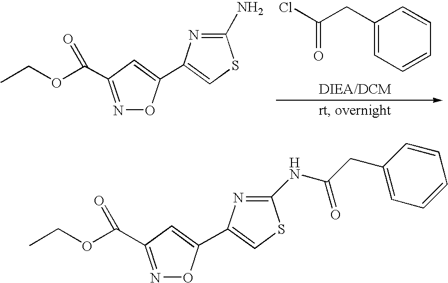

Proceeding as described in Example 1 above but using appropriate starting materials, the titled compound was prepared. ¹H NMR (DMSO-d₆): δ 8.01 (s, 1H), 7.34-7.25 (m, 5H), 7.12 (s, 1H), 4.39 (q, J=7.1 Hz, 2H), 3.81 (s, 2H), 1.34 (t, J=7.1 Hz, 3H); ¹³C NMR (DMSO-d₆): δ 169.9, 166.8, 159.1, 156.6, 136.5, 134.6, 129.2, 128.4, 126.9, 115.2, 100.8, 99.5, 62.0, 41.6, 13.9; ESIMS (m/z) 358 (M+1)

Step 3

Synthesis of 5-(2-Phenylacetylamino-thiazol-4-yl)-isoxazole-3-carboxylic acid hydroxyamide

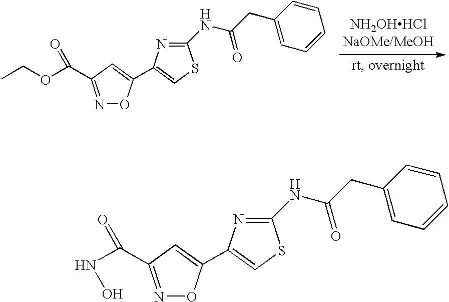

Proceeding as described in Example 1 above but using appropriate starting materials, the titled compound was prepared. ¹H NMR (DMSO-d₆): δ 12.76 (s, 1H), 11.62 (s, 11H), 9.47 (s, 1H), 7.94 (s, 1H), 7.37-7.24 (m, 5H), 6.99 (s, 1H), 3.81 (s, 2H); ¹³C NMR (DMSO-d₆):δ 169.9, 165.9, 159.1, 157.9, 155.8, 136.7, 134.6, 129.2, 128.4, 126.9, 114.8, 99.7, 41.6; ESIMS (m/z) 345 (M+1)

### EXAMPLE 18

### Preparation of 5-(3-Benzoylamino-ihenyl)-1H-pyrazole-3-carboxylic acid hydroxyamide

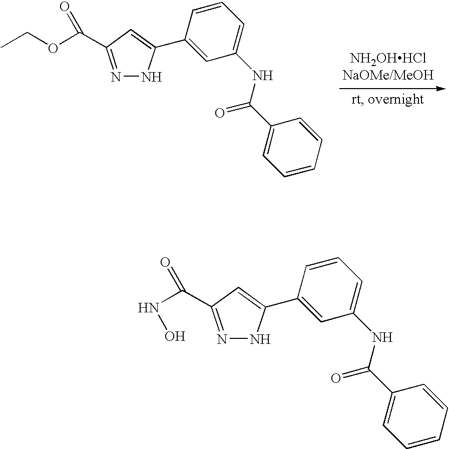

Proceeding as described in Example 1 above but using appropriate starting materials, the titled compound was prepared. ¹H NMR (CD₃OD): δ 8.18 (s, 1H), 8.05-8.02 (m, 2H), 7.77-7.51 (m, 6H), 7.10 (s, 1H); ESIMS (m/z) 367 (M+1)

### EXAMPLE 19

### Preparation of 5-[3-(2-Phenoxy-acetylamino)-phenyl]-1H-pyrazole-3-carboxylic acid hydroxyamide

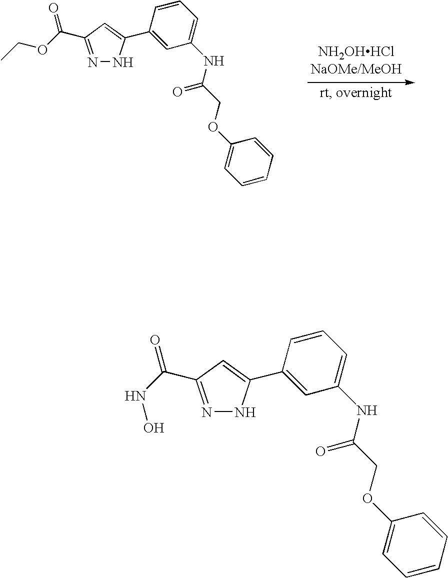

Proceeding as described in Example 1 above but using appropriate starting materials, the titled compound was prepared. ¹H NMR (DMSO-d₆): δ 11.17 (s, 1H), 10.17 (s, 1H), 8.12 (s, 1H), 7.60-7.30 (m, 5H), 7.04-6.97 (m, 4H), 4.73 (s, 2H); ESIMS (m/z) 353 (M+1)

### EXAMPLE 20

### Preparation of 5-(3-Benzenesulfonylamino-phenyl)-1H-pyrazole-3-carboxylic acid hydroxyamide

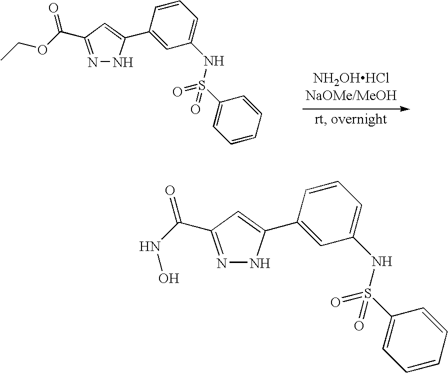

Proceeding as described in Example 1 above but using appropriate starting materials, the titled compound was prepared. ¹H NMR (DMSO-d₆): δ 11.17 (s, 1H), 10.30 (s, 1H), 8.06 (s, 1H), 7.53-7.23 (m, 8H), 6.99 (m, 1H); ESIMS (m/z) 359 (M+1)

### EXAMPLE 21

### Preparation of 5-(3-Phenylmethanesulfonylamino-phenyl)-1H-pyrazole-3-carboxylic acid hydroxyamide

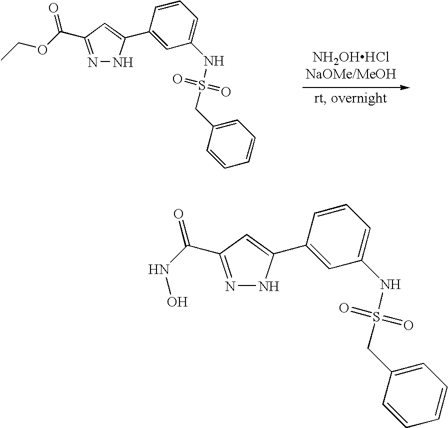

Proceeding as described in Example 1 above but using appropriate starting materials, the titled compound was prepared. ¹H NMR (DMSO-d₆): δ 11.19 (s, 1H), 9.95 (s, 1H), 7.60 (s, 1H), 7.46-7.14 (m, 8H), 7.01 (s, 1H), 4.51 (s, 2H); ESIMS (m/z) 387 (M+1)

### EXAMPLE 22

### Preparation of 5-[2-(3-Phenyl-pronylamino)-thiazol-4-yl]-thiophene-2-carboxylic acid hydroxyamide

Step 1

Synthesis of 5-[2-(3-Phenyl-propylamino)-thiazol4-yl]-thiophene-2carboxylic acid methyl ester

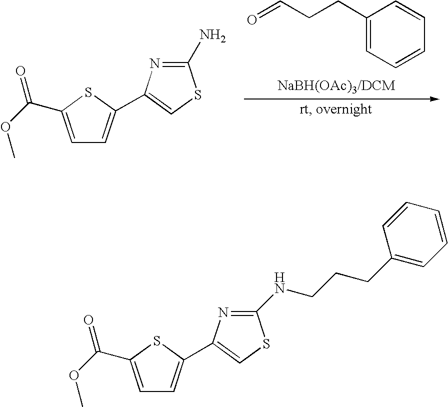

A mixture of 5-(2-Amino-thiazol-4-yl)-thiophene-2-carboxylic acid methyl ester (120 mg) and 3-Phenyl-propionaldehyde (79 μL) in DCM (4 mL) and AcOH (0.5 mL) was treated by NaBH(OAc)₃ (211.9 mg) at room temperature. The reaction was stirred at room temperature for overnight. The reaction was quenched by cold water and extracted by DCM. The organic layer was washed by sat. aq. sodium bicarbonate and brine and dried in anhydrous sodium sulfate. The organic layer was concentrated in vacuo. to afford the crude product which was used directly without purification. ESIMS (m/z) 359 (M+1)

Step 2

Synthesis of 5-[2-(3-Phenyl-propylamino)-thiazol-4-yl]-thiophene-2-carboxylic acid hydroxyamide

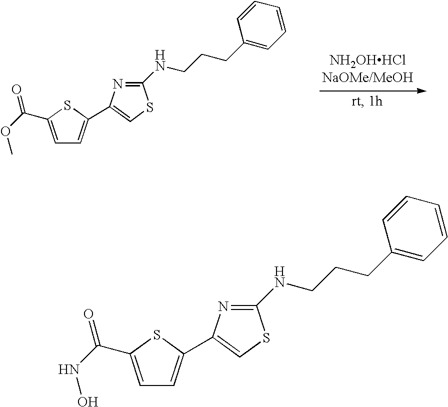

Proceeding as described in Example 1 above but using appropriate starting materials, the titled compound was prepared. ¹H NMR (CD₃OD): δ 7.32-6.87 (m, 8H), 3.31 (t, J=7.0 Hz, 2H), 2.68 (t, J=7.6 Hz, 2H), 1.95 (q, J=7.3 Hz, 2H); ESIMS (m/z) 360 (M+1)

Scheme II illustrates the procedure used for preparing compounds of Formula (I), wherein Z is a double bond. Compounds of Formula (I) can be prepared by analogous procedure, for example, by the choice of appropriate starting material through either Heck reaction or Wittig reaction to construct the double bond. For example, in the case of A is phenyl ring in Formula (I), such compound(s) can be synthesized by analogous method of Heck reaction illustrated in Scheme II starting with appropriate phenyl bromide, appropriate acrylate component (e.g. ethyl acrylate) and appropriate hydroxylamine or N-alkyl hydroxylamine (NHR₁OH where R, is defined as above); In case of A is furan ring and B is phenyl ring in Formula (I), such compounds can be synthesized by analogous method of Wittig reaction of appropriate aldehyde illustrated in Scheme II.

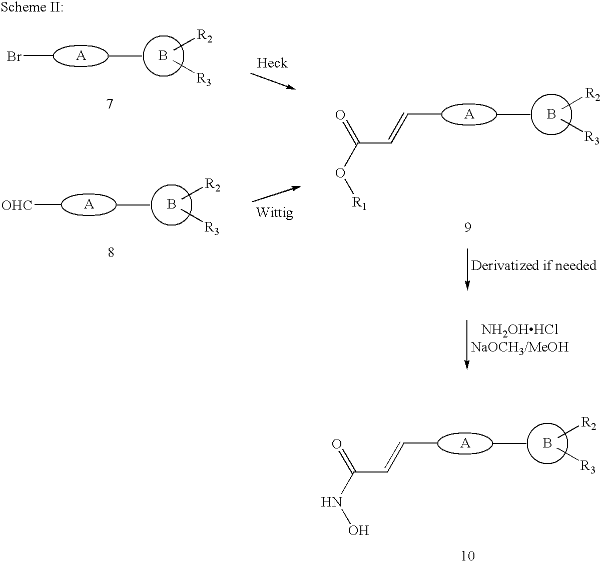

Specifically, the hydroxamate compounds Formula (I) (Z=—CH═CH—) of the present invention can be synthesized by the synthetic route shown in Scheme II. The double bond was introduced either through Heck reaction of aromatic bromide with appropriate acrylate or Wittig reaction of aldehyde with appropriate Wittig reagent. The resulting α,β-unsaturated ester was further derivatized accordingly if needed. Eventually the hydroxamate compounds were obtained by a known synthesis method (J. Med. Chem., 2002, 45, 753-757).

The biaryl bromide (7) could be prepared as exemplified by Scheme III. By using the analogous reaction of Scheme II, the haloketone (11) was converted to either aminothiazole (13) or oxazole (15). Both compounds are ready for heck reaction.

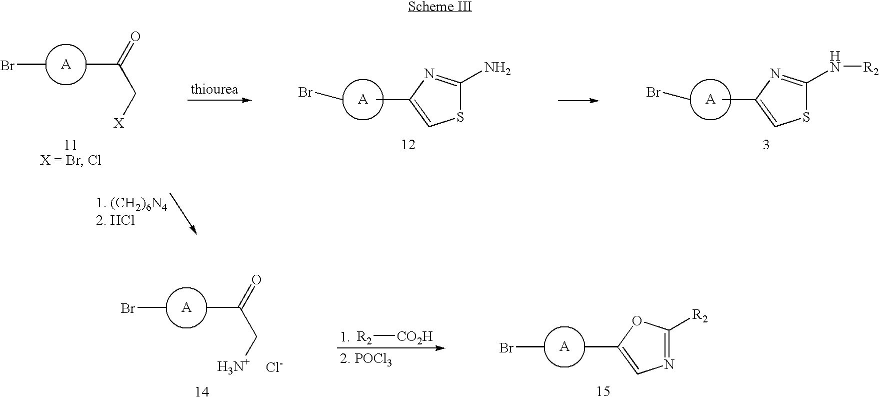

The following preparation and examples are given to enable those skilled in the art to more clearly understand and to practice the present invention. They should not be considered as limiting the scope of the invention, but merely as being illustrative and representative thereof.

### EXAMPLE 23

### Preparation of 3-[4-(2-Amino-thiazol-4-yl)-phenyl]-N-hydroxy-acrylamide

Step 1

Preparation of 4-(4-Bromo-phenyl)-thiazol-2-ylamine hydrobromide

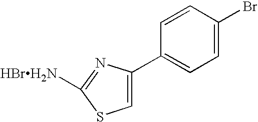

To a 100 mL round-bottomed flask, 4-bromophenacyl bromide (2.772 9, 9.97 mmol), thiourea (0.762 9, 10.0 mmol) and absolute ethanol (40 mL) were added. The mixture was stirred and heated in an oil batch at 80° C. for 140 min, then evaporated to dryness and white solids were obtained (3.347 g, 99.8%).

LC-MS (ESI, positive mode): m/z=255/257 [(M-Br)]⁺¹H NMR (DMSO-d₆) δ 7.72 (4H, s), 7.33 (1H, s), 3.8-4.3 (3H, bs, NH₃+); ¹³C NMR (DMSO-d₆) δ 170.0, 139.7, 131.9 (CH×2), 128.9, 127.8 (CH×2), 122.2, 103.6 (CH).

Step 2

Preparation of 3-[4-(2-Amino-thiazol-4-yl)-phenyl]-acrylic acid ethyl ester

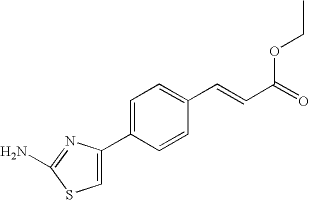

To a 50 mL round-bottomed flask, 4-(4-Bromo-phenyl)-thiazol-2-ylamine hydrobromide (0.522 g, 1.55 mmol), triphenylphosphine (0.072 g, 0.277 mmol), tetrakis(triphenylphosphine)palladium (0) (0.069 g, 0.059 mmol), DMF (5 mL), i-Pr₂NEt (0.80 mL, 4.59 mmol) and ethyl acrylate (0.35 mL, 3.22 mmol) were added. The above mixture was heated in an oil bath at 100° C. for 50.5 h under N₂. The mixture was diluted with EtOAc and aqueous NaHCO₃, then extracted with EtOAc twice. The extract was dried (Na₂SO₄) and concentrated to give an oil which was purified by flash chromatography (silica, 50% EtOAc in hexanes). 3-[4-(2-Amino-thiazol-4-yl)-phenyl]-acrylic acid ethyl ester was obtained as white yellow solid (0.132 g, 31%)

LC-MS (ESI, positive mode): 275 [(M+H)]⁺¹H NMR (CDCl₃) δ 7.76 (2H, d, J=8.4 Hz), 7.67 (1H, d, J=16.0 Hz), 7.51 (2H, d, J=8.3 Hz), 6.77 (1H, s), 6.43 (1H, d, J=16.0 Hz), 5.40 (2H, s), 4.26 (2H, q, J=7.1 Hz), 1.34 (3H, t, J=7.1 Hz); ¹³C NMR (CDCl₃) δ 167.2 (SC(═N)NH₂), 166.6 (CO₂), 150.0, 143.6, 135.9, 133.3, 127.9, 125.9, 117.5, 103.5, 60.0, 13.8 (CH₃).

Step 3

Preparation of 3-[4-(2-Amino-thiazol-4-yl)-phenyl]-N-hydroxy-acrylamide

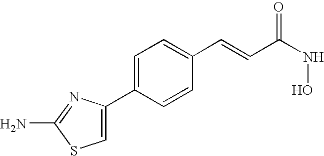

To a 50 mL round-bottomed flask, 3-[4-(2-Amino-thiazol-4-yl)-phenyl]-acrylic acid ethyl ester (17.6 mg, 0.0642 mmol) and hydroxylamine hydrochloride (46.3 mg, 0.616 mmol) were added. Anhydrous methanol (0.5 mL) was added into the flask via syringe under N₂ and then followed by sodium methoxide solution (5.38 M, 0.16 mL, 0.86 mmol). The above mixture was stirred at room temperature for 4 h and quenched by addition of 1N HCl and EtOAc. The solution was extracted with EtOAc twice (mainly acid by LC-MS) and the aqueous phase (mainly the product by LC-MS) was subjected to reverse-phase preparative HPLC (C18, 20×180 mm, 20 mL/min, 5 to 45% acetonitrile+0.1% TFA in 20 min). 3-[4-(2-Amino-thiazol-4-yl)-phenyl]-N-hydroxy-acrylamide was obtained as pale yellow power (6.0 mg as TFA salt, 25%).

LC-MS (ESI, positive mode): 262 [(M+H)]⁺¹H NMR (DMSO-d₆) δ 10.76 (s, residual H after exchanging with water), 7.81 (2H, d, J=8.3 Hz), 7.59 (2H, d, J=8.3 Hz), 7.45 (1H, d, J=15.8 Hz), 7.16 (1H, s), 6.48 (1H, d, J=15.8 Hz); ¹³C NMR (DMSO-d₆) δ 168.8, 162.6 (CONHOH), 146.3, 137.7, 136.9, 134.1, 127.8, 126.0, 119.0, 102.9.

### EXAMPLE 23A

### Preparation of 3-[4-(2-Amino-thiazol-4-yl)-phenyl]-N-hydroxy-acrylamide hydrochloride salt The 3-[4-(2-Amino-thiazol-4-yl)-phenyl]-N-hydroxy-acrylamide TFA salt was dissolved in MeOH/DCM and basified with 1N KOH to form precipitates. The precipitates were washed with water twice, then dissolved in MeOH/DCM by adding 6N HCl to pH˜1. The solution was evaporated to dryness to give the titled compound. ¹H NMR (DMSO-d₆) δ: 10.80 (s, b, residual H after exchanging with water), 7.80 (2H, d, J=8.3 Hz), 7.60 (2H, d, J=8.0 Hz), 7.46 (1H, d, J=15.8 Hz), 7.16 (1H, s), 6.49 (1H, d, J=15.9 Hz).

### EXAMPLE 24

### Preparation of 3-[4-(2-Amino-thiazol4-yl)-phenyl]-N-hydroxy-acrylamide

step 1

Preparation of 3-[4-(2-Acetylamino-thiazol-4-yl)-phenyl]-acrylic acid ethyl ester

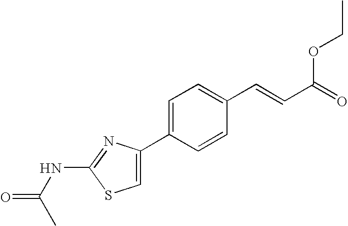

To a 50 mL round-bottomed flask, 3-[4-(2-Amino-thiazol-4-yl)-phenyl]-acrylic acid ethyl ester (21.8 mg, 0.080 mmol) was added and then followed by dichloromethane (1.2 mL), acetic anhydride (0.0375 mL, 0.40 mmol) and triethylamine (0.10 mL, 0.72 mmol) under N₂. The solution was stirred at room temperature for 4 days and then diluted by addition of dichloromethane. The resultant solution was and filtered through a pad of silica. The silica was washed with 33% EtOAc in hexanes and pure EtOAc respectively. 3-[4-(2-Acetylamino-thiazol-4-yl)-phenyl]-acrylic acid ethyl ester was obtained as yellow solid (22.8 mg, 91%).

LC-MS (ESI, positive mode): 317 [(M+H)]⁺¹H NMR (CDCl₃) δ 10.91 (1H, bs, NH), 7.81 (2H, d, J 8.4 Hz), 7.70 (1H, d, J=16.0 Hz), 7.56 (2H, d, J=8.3 Hz), 7.20 (1H, s), 6.49 (1H, d, J=16.0 Hz), 4.28 (2H, q, J=7.1 Hz), 1.99 (3H, s, Ac), 1.35 (3H, t, J=7.1 Hz, CH₃); ¹³C NMR (CDCl₃) δ 167.8, 166.5, 158.7, 148.2, 143.4, 135.3, 133.7, 128.1, 126.1, 17.9, 108.5, 60.1, 22.4, 13.8 (CH₃).

Step 2

Preparation of 3-[4-(2-Acetylamino-thiazol-4-yl)-phenyl]-N-hydroxy-acrylamide

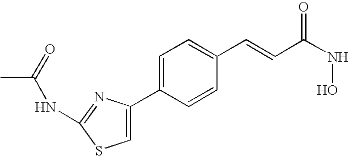

Proceeding as described in Example 22 above but using appropriate starting materials, the titled compound was prepared (2.4 mg obtained from 20 mg of 3-[4-(2-Amino-thiazol-4-yl)-phenyl]-acrylic acid ethyl ester). LC-MS (ESI, positive mode): 304 [(M+H)]⁺

### EXAMPLE 25

### Preparation of 3-[5-(3-Chloro-phenyl)-furan-2-yl]-N-hydroxy-acrylamide

Step 1

Synthesis of 3-[5-(3-Chloro-phenyl)-furan-2-yl]-acrylic acid methyl ester

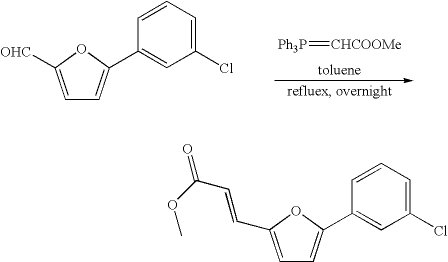

A solution of 213 μL 5-(3-Chloro-phenyl)-furan-2-carbaldehyde in 6 mL toluene was treated by 801.6 mg (Triphenyl-15-phosphanylidene)-acetic acid methyl ester at room temperature. The reaction was heated to reflux for overnight. The reaction was cooled to room temperature and concentrated in vacuo. The crude product was purified by flash chromatography on silica gel to afford the desired product 380.2 mg (94%).

LC-MS (ESI, positive mode): 263 [(M+H)]⁺¹H NMR (CDCl₃) δ 7.71-7.59 (2H, m), 7.47 (1H, d, J=15.7 Hz), 7.38-7.29 (3H, m), 6.76 (1H, d, J=3.6 Hz), 6.70 (1H, d, J=3.6 Hz), 6.45 (1H, d, J=15.7 Hz), 3.83 (3H, s); ¹³C NMR (CDCl₃) δ 166.9, 154.0, 150.3, 134.4, 131.0, 130.2, 129.6, 127.8, 123.8, 121.9, 116.6, 115.1, 108.2, 51.2.

Step 2

Preparation of 3-[5-(3-Chloro-phenyl)-furan-2-yl]-N-hydroxy-acrylamide

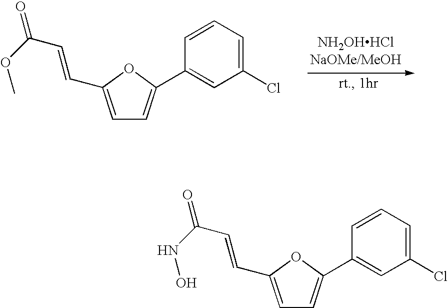

Proceeding as described in Example 1 above but using appropriate starting materials, the titled compound was prepared.

LC-MS (ESI, positive mode): 264 [(M+H)]⁺

Scheme IV illustrates the procedure used for preparing compounds of Formula (I), wherein RₐNHR_{b} (Rₐ, R_{b} are independently selected from R₆ or R₇ as defined above) is either an amine made in-house (by reductive amination or alkylation) or a commercial available product. Compounds of Formula (I) can be prepared by analogous procedure, for example, by the choice of appropriate starting material. For example, in the case of A is phenyl and B is thiophene in Formula (I), such compound(s) (18) can be synthesized by analogous method illustrated in Scheme IV starting with 5-(4-Formyl-phenyl)-thiophene-2-carboxylic acid methyl ester, and appropriate amine component, and appropriate hydroxylamine or N-alkyl hydroxylamine (NHR₁OH where R₁ is defined as above). The 2^{nd} amine (17, R_{b}=H) could be converted to a tertiary amine hydroxamates (19) by a 2^{nd} reductive amination with aldehyde R_{c}CHO (R_{c} is selected from R₆ or R₇ as defined above) or to 20 by alkylation. Biaryl aldehyde (16) could be prepared by a Suzuki coupling reaction between a suitable bromide (ring A) and boronic acid (ring B). Such a reaction was exemplified by the preparation of INTERMEDAITE 1.

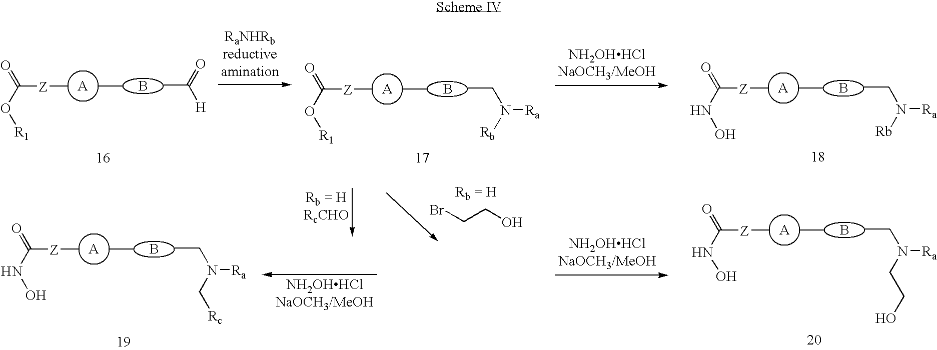

Specifically, the hydroxamate compounds in Examples 26 to 46 of the present invention can be synthesized by the synthetic route shown in Scheme IV.

The following preparation and examples are given to enable those skilled in the art to more clearly understand and to practice the present invention. They should not be considered as limiting the scope of the invention, but merely as being illustrative and representative thereof.

### INTERMEDIATE 1

### Preraration of 4-(4-Formyl-phenyl)-thiophene-2-carboxylic acid methyl ester

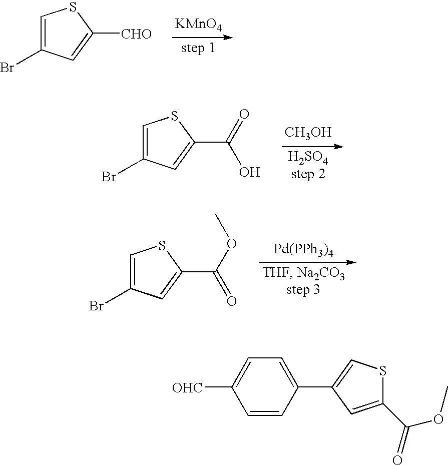 Step 1

The mixture of 4-bromothiophene-2-carboxaldehyde (15 g), KMnO₄ (13.5 g), H₂O (500 Ml) and NaOH (5 g) was stirred overnight, then filtered. The filtrate was acidified with aq. HCl and extracted with EtOAc. The organic layer was washed with water and brine, then dried over Na₂SO₄, and concentrated to give 4-Bromo-thiophene-2-carboxylic acid (14.2 g).

Step 2

4-Bromo-thiophene-2-carboxylic acid (12.85 g), CH₃OH (360 mL) and H₂SO₄ (95˜98%, 6 mL) were refluxed overnight. The solution was basified and evaporated to remove the organic solvent. The residue was extracted with EtOAc. The organic layer was washed with water and brine, then dried over Na₂SO₄, evaporated to give the product the solvent gave the product 4-Bromo-thiophene-2-carboxylic acid methyl ester (13 g).

Step 3

4-Bromo-thiophene-2-carboxylic acid methyl ester (8.67 g), 4-Formylphenylboronic acid (13 g), Pd(PPh₃)₄ (2.08 g), THF (100 mL), Na₂CO₃ aqueous solution (100 mL, 2M) were refluxed overnight (90˜100° C.), then extracted the reaction mixture with EtOAc, and washed the organic layer by 5% NaOH solution, followed by water and brine, then dried over Na₂SO₄, After evaporation, the residue was washed with Et₂O and afforded 4-(4-Formyl-phenyl)-thiophene-2-carboxylic acid methyl ester (7 g).

### EXAMPLE 26

### Preparation of 5-[4-(Phenethylamino-methyl)-phenyl]-thiorhene-2-carboxylic acid hydroxyamide

Step 1

Synthesis of 5-[4-(Phenethylamino-methyl)-phenyl]-thiophene-2-carboxylic acid methyl ester

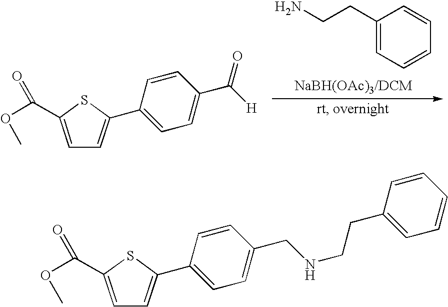

A mixture of 5-(4-Formyl-phenyl)-thiophene-2-carboxylic acid methyl ester (271 mg) and 2-Phenylethylamine (126 μL) in DCM (4 mL) was treated by NaBH(OAc)₃ (318 mg) at room temperature. The reaction was stirred at room temperature for overnight. The reaction was quenched by cold water and extracted by DCM. The organic layer was washed by sat. aq. sodium bicarbonate and brine and dried in anhydrous sodium sulfate.

The organic layer was concentrated in vacuo to afford the crude product which was used directly without purification. ESIMS (m/z) 352 (M+1)

Step 2

Preparation of 5-[4-(Phenethylamino-methyl)-phenyl]-thiophene-2-carboxylic acid hydroxyamide.

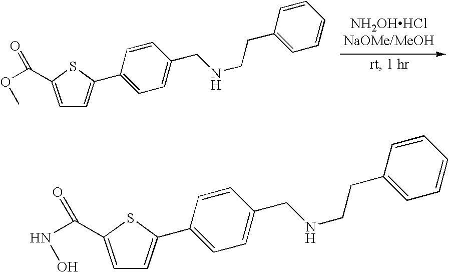

Proceeding as described in Example 1 above but using appropriate starting materials, the titled compound was prepared as TFA salt. ESIMS (m/z) 353 (M+1). ¹H NMR (CD₃OD): δ 7.72 (d, J=8.2 Hz, 2H), 7.49 (d, J=8.3 Hz), 7.49-7.19 (m, 7H), 4.20 (s, 2H), 3.25 (m, 2H), 2.97 (m, 2H).

### EXAMPLE 26A

### Freebase of Example 26

The preparative HPLC fractions (aqueous acetonitrile with 0.1% TFA) containing 5-[4-(Phenethylamino-methyl)-phenyl]-thiophene-2-carboxylic acid hydroxyamide were combined and basified with 1M NaOH to PH 9˜10, and the solid was filtered and washed with water to give the freebase of Example 26 as yellow solid. HPLC purity (at 254 nm)=99.2%. ¹H NMR (DMSO-d₆) δ 7.64 (d, 2H,J=8.1 Hz), 7.59 (br s-like, 1H), 7.48 (d, 2H, J=3.9 Hz), 7.39 (d, 2H, J=8.2 Hz), 7.27 (t, 2H, J=7.3 Hz), 7.21-7.15 (m, 3H), 3.78 (s, 2H), 2.76 (s, 4H).

### EXAMPLE 26B

### Mesylate of Example 26.

Example 26A (1.4 g, 3.98 mmol) was suspended in a mixed solvent (MeOH:DCM=2:1, 375 mL). The resulting solution was added methanesulfonic acid (0.46 g, 4.79 mmoL, 1.2 eq). The above solution was sonicated for 2-3 min then stirred at room temperature for 1 hour. After being concentrated to about 50 mL under reduced pressure, the white solid formed was filtered, washed with EtOAc and methanol to remove the excess methanesulfonic acid. The ttle compound was obtained as white solids (1.7 g, 96%). HPLC purity (at 254 nm)=99.7%. The proton NMR indicated that the ratio of Example 26: methanesulfonic acid is 1:1. ¹H NMR (DMSO-d₆) δ 11.28 (br s, 0.9 H), 9.18 (br s, 1H), 8.89 (br s) and 9.2-8.8 (very br, total 1.6H), 7.79 (d, 2H, J=8.2 Hz), 7.63 (br s, 1H), 7.58 (d, 1H, J=3.8 Hz), 7.57 (d, 2H, J=8.4 Hz), 7.35 (t, 2H, J=7.1 Hz), 7.30-7.20 (m, 3H), 4.22 (s, 2H), 3.18 (dd or m, 2H), 2.97 (dd or m, 2H), 235 (s, 3H, Me); ¹³C NMR (DMSO-d₆) δ 159.2, 146.1, 137.1, 136.8, 133.6, 132.1, 130.8, 128.7, 128.6, 18.5 (br, confirmed by ¹H-¹³C HSQC), 126.8, 125.8, 124.9, 49.7, 47.7, 39.7 (Me), 31.6.

The following compounds are prepared by methods analogous to those disclosed in Examples 26

TABLE 2
Representative Examples made by methods described in Scheme IV.

| | | m/z | |
|---|---|---|---|
| Example | Structures | [MH]⁺ | NMR |
| 27 | 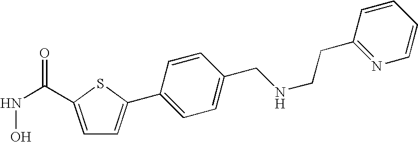 | 354 | ¹H NMR (CD₃OD): δ 8.56 (m, 1H, —Ar—H), 8.0 (m, 1H), 7.70 (d, J=8.3 Hz, 2H), 7.53-7.46 (m, 5H), 7.38 (d, J=3.9 Hz, 1H), 4.27 (s, 2H), 3.46 (t, J=7.3 Hz, 2H), 3.28 (t, J=7.1 Hz, 2H); ¹³C NMR (CD₃OD) δ155.2, 146.4, 139.4, 134.1, 130.6, 129.9, 128.6, 125.7, 124.0, 123.7, 122.7, 117.3, 114.4, 49.8, 45.2, 30.8. |
| 28 | 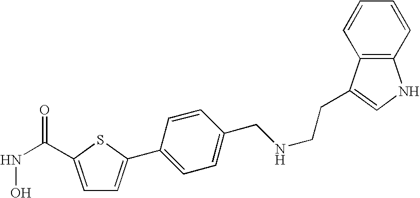 | 392 | ¹H NMR (CD3OD): δ 7.68 (d, J=8.2 Hz, 2H), 7.51-7.39 (m, 5H), 7.31 (d, J=8.1 Hz, 1H), 7.12 (s, 1H), 7.06 (m, 1H), 6.98 (m, 1H), 4.19 (s, 2H), 3.31 (t, J=7.3 Hz, 2H), 3.14 (t, J=7.4 Hz, 2H) |
| 29 | 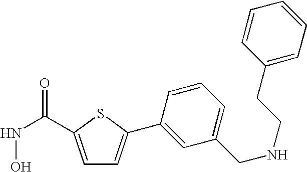 | 353 | ¹H NMR (CD3OD): δ 7.78 (br, 1H), 7.70 (d, J=7.6 Hz, 1H), 7.49-7.19 (m, 9H), 4.23 (s, 2H), 3.25 (m, 2H), 2.98 (t, J=8.7 Hz, 2H). |
| 30 | 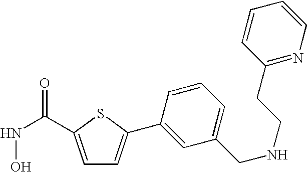 | 354 | ¹H NMR (CD3OD): δ 8.57 (m, 1H), 8.06 (m, 1H), 7.79 (s, 1H), 7.67-7.36 (m, 7H), 4.30 (s, 2H), 3.50 (m, 2H), 3.25 (m, 2H). |
| 31 | 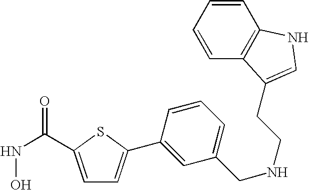 | 392 | ¹H NMR (CD₃OD): δ 7.29 (s, 1H), 7.67 (d, J=7.7 Hz, 1H), 7.49-7.29 (m, 6H), 7.12 (s, 1H, —Ar—H), 7.05 (m, 1H), 6.96 (m, 1H), 4.21 (s, 2H), 3.32 (t, J=7.3 Hz, 2H), 3.14 (t, J=7.4 Hz, 2H). |
| 32 | 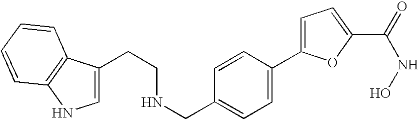 | 376 | HPLC purity at 254 nm: 100%; LC- MS (ESI, positive mode) m/z 379 ([M + H]⁺); ¹H NMR (CD₃OD) δ7.86-6.92 (m, 11H, Ar—H), 4.19 (s, 2H), 3.32-3.28 (t, 2H), 3.16-3.12 (t, 2H); ¹³C NMR (CD₃OD) δ 157.3, 154.5, 144.6, 136.3, 130.7, 130.1, 126.1, 108.1, 129.6, 124.3, 122.3, 120.9, 118.2, 116.9, 115.5, 110.6, 107.0, 49.9, 21.3. |
| 33 | 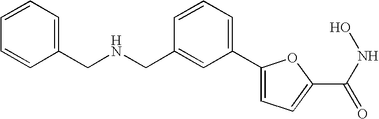 | 322 | HPLC purity at 254 nm: 100%; LC- MS (ESI, positive mode) m/z 323 ([M + H]⁺); ¹H NMR (CD₃OD) δ7.88-7.84 (m, 2H, Ar—H), 7.46-7.37 (m, 8H, Ar—H), 7.09 (d, 1H), 6.91-6.90 (d, 1H), 4.20-4.19 (d, 4H); ¹³C NMR (CD₃OD) δ 154.4, 144.7, 131.5, 130.5, 129.9, 128.9 (Ar—C), 129.2, 129.1, 128.9, 125.1, 124.7, 106.9, 50.3, 50.0. |
| 34 | 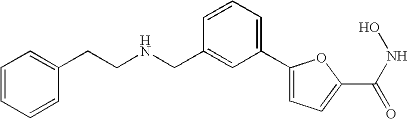 | 337 | HPLC purity at 254 nm: 99%; LC- MS (ESI, positive mode) m/z 337 ([M + H]⁺); ¹H NMR (CD₃OD) δ7.87-7.23 (m, 9H, Ar—H), 7.19-7.17 (d, 1H), 6.90-6.89 (d, 1H), 4.20 (s, 2H), 2.96-2.92 (t, 2H); ¹³C NMR (CD₃OD) δ 154.0, 144.0, 135.6, 131.4, 130.0 (Ar—C), 129.1, 128.1, 127.7, 126.4, 125.0, 124.8, 106.8, 50.2, 31.3. |
| 35 | 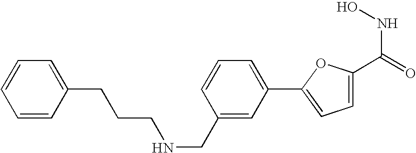 | 351 | HPLC purity at 254 nm: 100%; LC- MS (ESI, positive mode) m/z 351 ([M + H]⁺); ¹H NMR (CD₃OD) δ7.84-7.08(m, 10H, Ar—H), 6.88-6.87 (d, 1H, furan-H), 4.15-4.10 (d, 2H), 3.00-2.96 (t, 2H), 2.65-2.61 (t, 2H), 1.99-1.91 (m, 2H); ¹³C NMR (CD₃OD) δ 154.4, 144.7, 131.5, 130.5, 129.9, 128.9, 129.2, 129.1, 128.9, 125.1, 124.7, 106.9, 50.3, 50.0 |
| 36 | 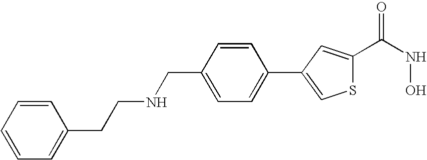 | 353 | HPLC purity at 254 nm: 100%; LC- MS (ESI, positive mode) m/z 353 ([M + H]⁺); ¹H NMR (CD₃OD) δ7.91-7.14 (m, 11H, Ar—H), 4.16 (s, 2H), 2.94-2.91 (t, 2H); ¹³C NMR (CD₃OD) δ 154.5, 139.5, 131.4, 130.0, 99.5, 129.0, 128.9, 127.7, 127.4, 125.5, 124.9, 124.7, 115.2 106.8, 50.1, 31.6, 26.9. |
| 37 | 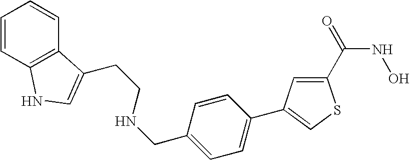 | 392 | HPLC purity at 254 nm: 95%; LC- MS (ESI, positive mode) m/z 392 ([M + H]⁺); ¹H NMR (CD₃OD) δ7.83-6.91 (m, 11H, Ar—H), 4.15 (s, 2H), 3.27 (t, 2H), 3.09 (t, 2H); ¹³C NMR (CD₃OD) δ 140.9, 136.4, 135.6, 108.1, 99.5, 129.7, 126.7, 125.9, 124.9, 122.2, 120.8, 118.1, 116.9, 110.6, 49.9, 21.3. |
| 38 | 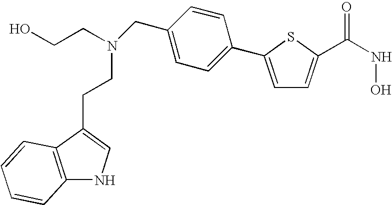 | 436 | ¹H NMR (CD₃OD): δ 7.63 (d, J=8.1 Hz, 2H), 7.5 (s, 1H), 7.4 (m, 3H), 7.30 (d, J=8.0 Hz, 1H), 7.26 (d, J=8.1 Hz, 2H), 7.11 (s, 1H), 7.00 (t, J=7.2 Hz, 1H), 6.87 (t, J=7.1 Hz, 1H), 4.11 (s, 2H), 3.90 (m, 2H), 3.41 (m, 4H), 3.20 (m, 2H). |
| 39 | 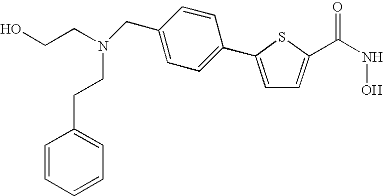 | 397 | ¹H NMR (CD₃OD): δ 7.76 (m, 3H), 7.56-7.43 (m, 3H), 7.28-7.18 (m, 5H), 4.48 (s, 2H), 3.87 (br, 2H), 3.39-3.29 (m, 4H), 3.07 (m, 2H) |
| 40 | 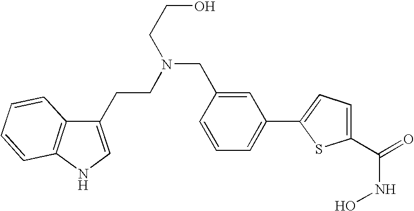 | 436 | ¹H NMR (CD₃OD): δ 7.69 (br, 1H), 7.64-7.60 (m, 1H), 7.50-7.48 (m, 1H), 7.38-7.23 (m, 6H), 7.23 (s, 1H), 7.09-7.01 (m, 1H), 6.98-6.89 (m, 1H), 4.52-4.41 (br, 2H), 3.90 (br, 2H), 3.48-3.37 (m, 4H), 3.20 (m, 2H). |
| 41 | 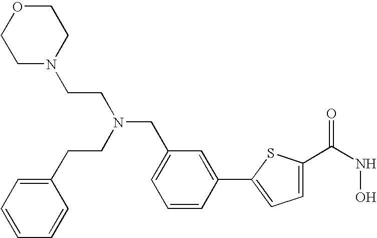 | 466 | ¹H NMR (CD₃OD): δ 7.82 (s, 1H, -Ph-H), 7.68 (d, J=6.2 Hz), 7.50-7.44 (m, 3H), 7.37 (d, J=3.8 Hz, 1H), 7.27-7.15 (m, 5H), 4.32 (s, 2H), 3.69 (br, 4H), 3.43-3.34 (m, 4H), 3.28-3.24 (m, 2H), 3.05-3.00 (m, 6H) |
| 42 | 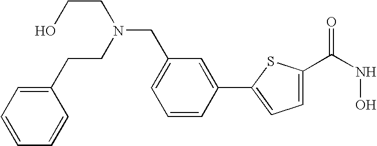 | 397 | ¹H NMR (CD₃OD): δ 7.85 (s, 1H), 7.76 (d, J=6.8 Hz), 7.53-7.42 (m, 3H), 7.41 (d, J=3.8 Hz, 1H), 7.27-7.15 (m, 5H), 4.53 (s, 2H), 3.89 (br, 2H), 3.39-3.29 (m, 4H), 3.08 (m, 2H) |
| 43 | 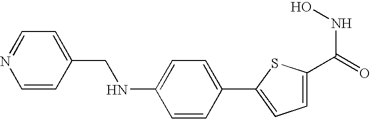 | 326 | ¹H NMR (CD₃OD): δ 8.66 (d, J=6.7 Hz, 2H), 7.97 (d, J=6.5 Hz, 2H), 7.45 (s, 1H), 7.21 (d, J=3.9 Hz, 1H), 7.00 (t, J=7.9 Hz, 1H), 6.91 (d, J=7.6 Hz, 1H), 6.79 (s, 1H), 6.52-6.50 (m, 1H), 4.66 (s, 2H) |
| 44 | 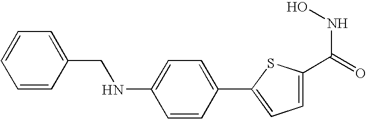 | 325 | ¹H NMR (CD₃OD): δ 7.47 (br, 1H), 7.35-7.09 (m, 7H), 7.05 (s, 1H), 6.78-6.76 (m, 1H), 4.38 (s, 2H) |
| 45 | 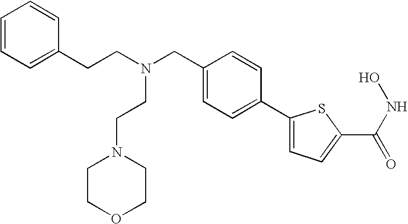 | 466 | ¹H NMR (CD₃OD): δ 7.67 (d, J=67.4 Hz, 2H), 7.61-7.41 (m, 3H), 7.36 (d, J=3.6 Hz, 1H), 7.31-7.15 (m, 5H), 4.34 (s, 2H), 3.76 (br, 4H), 3.47-3.43 (m, 4H), 3.28-3.24 (m, 2H), 3.08 (br, 6H) |
| 46 | 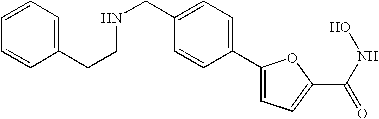 | 337 | HPLC purity at 254 nm: 100%; ¹H NMR (CD₃OD) δ 7.83 (d, 2H, J=8.4 Hz), 7.47 (d, 2H, J=8.4 Hz), 7.24 (t, 2H, J=6.2 Hz), 7.19-7.16 (m, 3H), 7.07 (br d, 1H, J=3.2 Hz), 6.89 (d, 1H, J=3.6 Hz), 4.17 (s, 2H), 3.20 (m, 2H), 2.93 (dd, 2H, J=8.6, 2.8 Hz); ¹³C NMR (CD₃OD) δ 157.19, 154.5, 144.6, 135.7, 130.7, 130.2, 129.6, 128.1, 127.7, 126.4, 124.3, 115.4, |
| # 107.0, 50.1, 31.3; ¹H NMR (DMSO-d₆) δ11.22 (s, 1H), 9.10 (s, 1H), 8.88 (s, 1H), 7.90 (d, 2H, J=8.3 Hz), 7.51 (d, 2H, J=8.4 Hz), 7.28 (tt- like, 2H, J=6.9, 1.0 Hz), 7.20 (td like, 2H, J=7.5, 1.2 Hz), 7.19 (d, 2H, J=8.3 Hz), 7.09 and 7.07 (each d, 1H, AB system, J=3.6 Hz), 4.16 (s, 2H), 3.12 (m, 2H), 2.88 (m, 2H). | | | |

### Synthesis of Biaryl Linked Hydroxamates by Parallel Synthesis

The synthetic method of Scheme IV could also be used for parallel synthesis. Instead of using methyl ester, a protected hydroxamate (21) was used for reductive amination with amine R₆NHR₇ (Scheme V). After TFA cleavage, the final products (23) were purified by reverse phase HPLC.

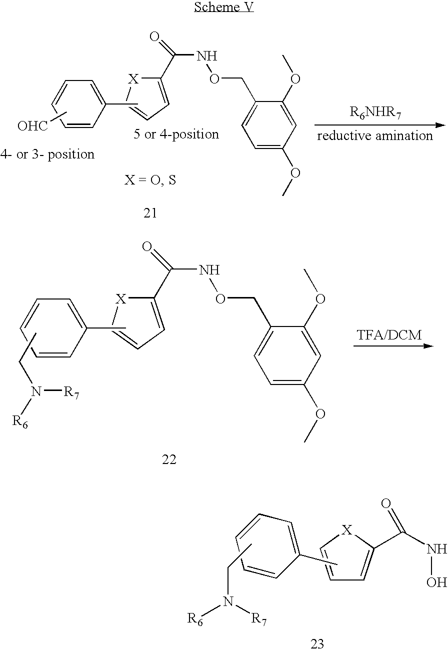

The protected hydroxamates (21) could be synthesized by the methods described in INTERMEDIATE 2 and INTERMEDIATE 3.

### INTERMEDIATE 2

### Preparation of 5-(4-Formyl-phenyl)-furan-2-carboxylic acid (2,4-dimethoxy-benzyloxy)-amide

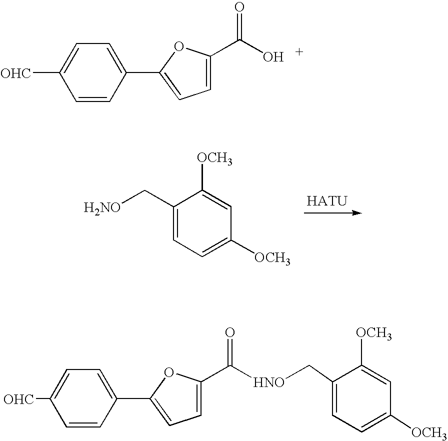

O-(7-Azabenzotriazol-1-yl)-N,N,N′,N′-tetramethyluronium hexafluorophosphate (HATU, 6.6 g) and 5-(4-Formyl-phenyl)-furan-2-carboxylic acid (3.14 g, was made by method analogous to INTERMEDIATE 1, but suing appropriate starting material and the methyl ester was hydrolysed to the acid) were added to the solution of O-(2,4-Dimethoxy-benzyl)-hydroxylamine (2.64 g) and DIEA (6.26 mL) in DMF (60 mL) at 0° C., and stirred at the same temperature for about 1 h. After the TLC showing the substances disappeared, saturated sodium bicarbonate was added to the reaction mixture, and stirred for additional 1 h, worked up to give a yellow oil. The oil was dissolved in small amount of THF, then diluted with water (the oil appeared again), under vigorous stirring ether was added, the oil solidified soon. The solid was filtered and washed with water and ether. The solid was recrystallized from methanol/ether to give 3.8 g of 5-(4-Formyl-phenyl)-furan-2-carboxylic acid (2,4-dimethoxy-benzyloxy)-amide.

### INTERMEDIATE 3

### Preparation of 5-(3-Formyl-phenyl)-furan-2-carboxylic acid (2,4-dimethoxy-benzyloxy)-amide

The title compound was made by method analogous to INTERMEDIATE 2.

Alternatively, it was also made by the following method. The acid (25) which was made by method analogous to INTERMEDIATE 1, was reacted with protected hydroxylamine (24) by using N, N′-Dicyclohexylcarbodiimide (DCC) as coupling reagent. The resulting bromide (26) was used for Suzuki coupling to give the title compound (28).

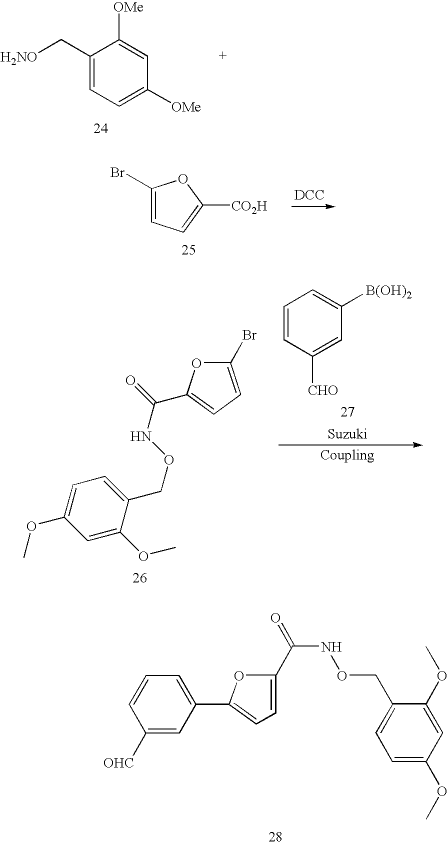

Parallel synthesis of 5-(4-(substituted aminomethyl-ohenyl)-furan-2-carboxylic acid hydroxyamide
Scheme VI

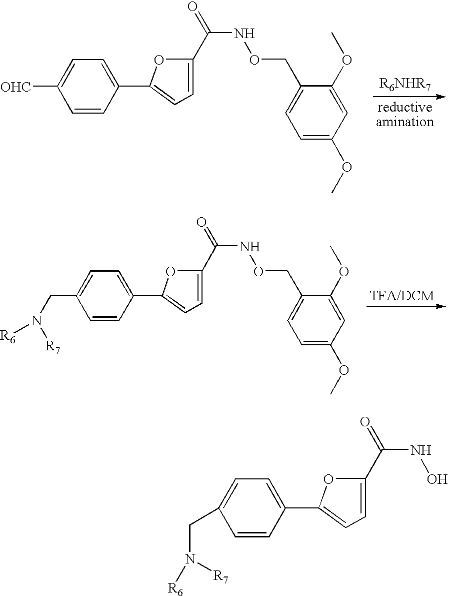

5-(4-Formyl-phenyl)-furan-2-carboxylic acid (2,4-dimethoxy-benzyloxy)-amide was reacted individually with 48 different amines (R₆NHR₇, 2 eq.) in DCM:MeOH (1:1), NaBH₃CN (1.5 eq.) and AcOH (1 eq.) overnight by using a 96-well plate. The organic solvent was removed by blowing the vial with nitrogen gas. The vials contained residue were added 95% TFA in DCM for cleavage (rt, 1 h). The solutions were dried and the residues were purified by high-throughput mass-dependent (reverse-phase HPLC) purification system (HTP). 45 compounds were collected.

TABLE 3
Examples made by parallel synthesis

| | | LC-MS | |
|---|---|---|---|
| Compound | Structure | (M + H) | Chemical Name |
| L01 | 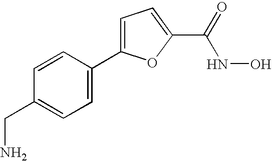 | 233 | 5-(4-Aminomethyl-phenyl)- furan-2-carboxylic acid hydroxyamide |
| L02 | 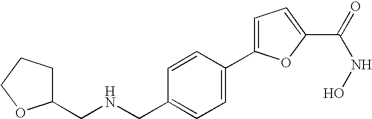 | 317 | 5-(4-{[(Tetrahydro-furan-2- ylmethyl)-amino]-methyl}- phenyl)-furan-2-carboxylic acid hydroxyamide |
| L03 | 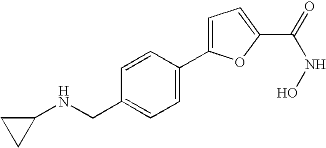 | 273 | 5-(4- Cyclopropylaminomethyl- phenyl)-furan-2-carboxylic acid hydroxyamide |
| L04 | 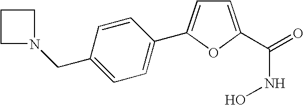 | 273 | 5-(4-Azetidin-1-ylmethyl- phenyl)-furan-2-carboxylic acid hydroxyamide |
| L05 | 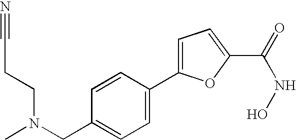 | 300 | 5-(4-{[(2-Cyano-ethyl)- methyl-amino]-methyl}- phenyl)-furan-2-carboxylic acid hydroxyamide |
| L06 | 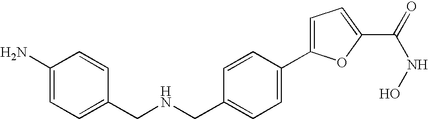 | 338 | 5-{4-[(4-Amino- benzylamino)-methyl]- phenyl}-furan-2-carboxylic acid hydroxyamide |
| L07 | 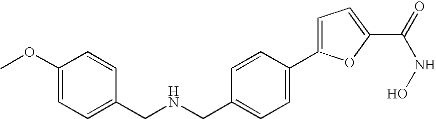 | 353 | 5-{4-[(4-Methoxy- benzylamino)-methyl]- phenyl}-furan-2-carboxylic acid hydroxyamide |
| L08 | 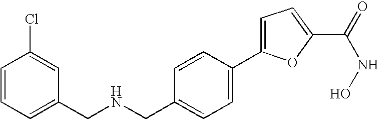 | 357 | 5-{4-[(3-Chloro- benzylamino)-methyl]- phenyl}-furan-2-carboxylic acid hydroxyamide |
| L09 | 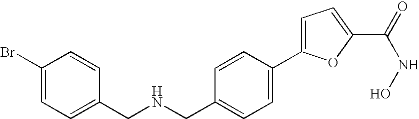 | 402 | 5-{4-[(4-Bromo- benzylamino)-methyl]- phenyl}-furan-2-carboxylic acid hydroxyamide |
| L10 | 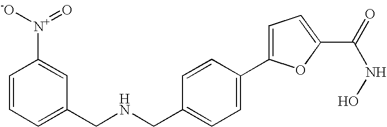 | 368 | 5-{4-[(3-Nitro-benzylamino)- methyl]-phenyl}-furan-2- carboxylic acid hydroxyamide |
| L11 | 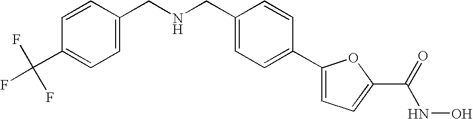 | 391 | 5-{4-[(4-Trifluoromethyl- benzylamino)-methyl]- phenyl}-furan-2-carboxylic acid hydroxyamide |
| L12 | 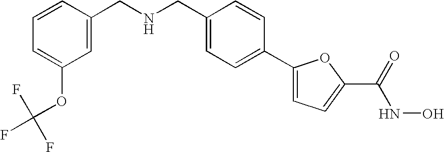 | 407 | 5-{4-[(3-Trifluoromethoxy- benzylamino)-methyl]- phenyl}-furan-2-carboxylic acid hydroxyamide |
| L13 | 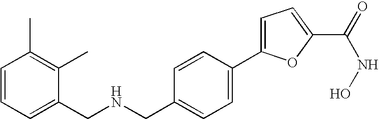 | 351 | 5-{4-[(2,3-Dimethyl- benzylamino)-methyl]- phenyl}-furan-2-carboxylic acid hydroxyamide |
| L14 | 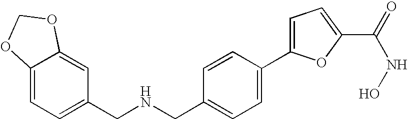 | 367 | 5-(4-{[(Benzo[1,3]dioxol-5- ylmethyl)-amino]-methyl}- phenyl)-furan-2-carboxylic acid hydroxyamide |
| L15 | 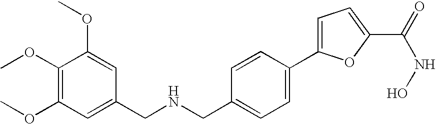 | 413 | 5-{4-[(3,4,5-Trimethoxy- benzylamino)-methyl]- phenyl}-furan-2-carboxylic acid hydroxyamide |
| L16 | 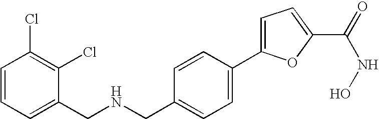 | 392 | 5-{4-[(2,3-Dichloro- benzylamino)-methyl]- phenyl}-furan-2-carboxylic acid hydroxyamide |
| L17 | 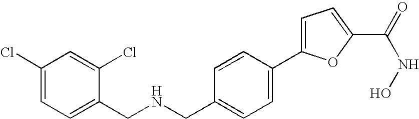 | 392 | 5-{4-[(2,4-Dichloro- benzylamino)-methyl]- phenyl}-furan-2-carboxylic acid hydroxyamide |
| L18 | 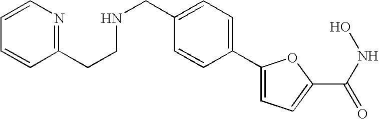 | 338 | 5-{4-[(2-Pyridin-2-yl- ethylamino)-methyl]- phenyl}-furan-2-carboxylic acid hydroxyamide |
| L19 | 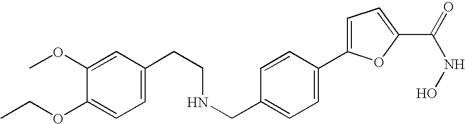 | 411 | 5-(4-{[2-(4-Ethoxy-3- methoxy-phenyl)- ethylamino]-methyl}- phenyl)-furan-2-carboxylic acid hydroxyamide |
| L20 | 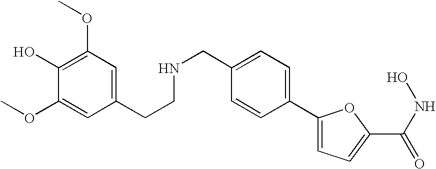 | 413 | 5-(4-{[2-(4-Hydroxy-3,5- dimethoxy-phenyl)- ethylamino]-methyl}- phenyl)-furan-2-carboxylic acid hydroxyamide |
| L21 | 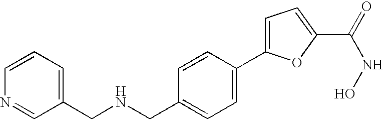 | 324 | 5-(4-{[(Pyridin-3-ylmethyl)- amino]-methyl}-phenyl)- furan-2-carboxylic acid hydroxyamide |
| L22 | 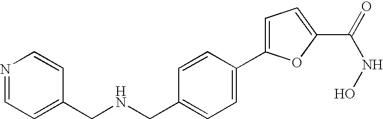 | 324 | 5-(4-{[(Pyridin-4-ylmethyl)- amino]-methyl}-phenyl)- furan-2-carboxylic acid hydroxyamide |
| L23 | 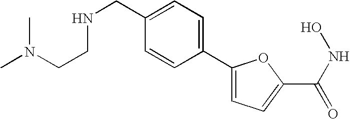 | 304 | 5-{4-[(2-Dimethylamino- ethylamino)-methyl]- phenyl}-furan-2-carboxylic acid hydroxyamide |
| L24 | 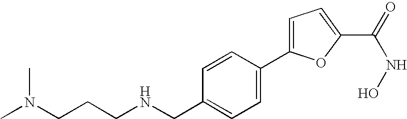 | 318 | 5-{4-[(3-Dimethylamino- propylamino)-methyl]- phenyl}-furan-2-carboxylic acid hydroxyamide |
| L25 | 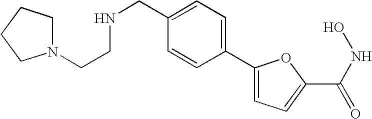 | 330 | 5-{4-[(2-Pyrrolidin-1-yl- ethylamino)-methyl]- phenyl}-furan-2-carboxylic acid hydroxyamide |
| L26 | 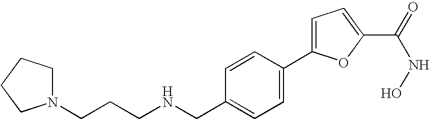 | 344 | 5-{4-[(3-Pyrrolidin-1-yl- propylamino)-methyl]- phenyl}-furan-2-carboxylic acid hydroxyamide |
| L27 | 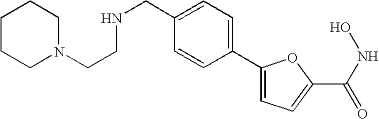 | 344 | 5-{4-[(2-Piperidin-1-yl- ethylamino)-methyl]- phenyl}-furan-2-carboxylic acid hydroxyamide |
| L28 | 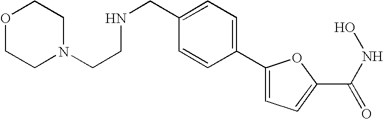 | 346 | 5-{4-[(2-Morpholin-4-yl- ethylamino)-methyl]- phenyl}-furan-2-carboxylic acid hydroxyamide |
| L29 | 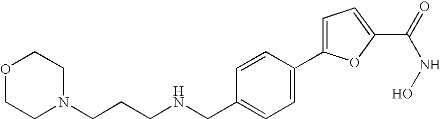 | 360 | 5-{4-[(3-Morpholin-4-yl- propylamino)-methyl]- phenyl}-furan-2-carboxylic acid hydroxyamide |
| L30 | 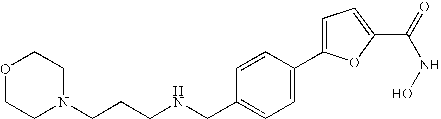 | 318 | 5-{4-[(3-Morpholin-4-yl- propylamino)-methyl]- phenyl}-furan-2-carboxylic acid hydroxyamide |
| L31 | 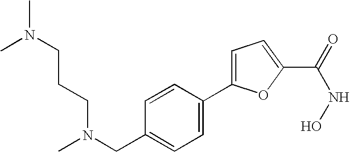 | 332 | 5-(4-{[(3-Dimethylamino- propyl)-methyl-amino]- methyl}-phenyl)-furan-2- carboxylic acid hydroxyamide |
| L32 | 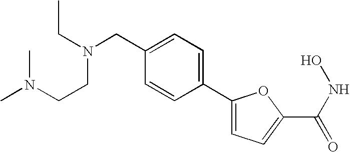 | 332 | 5-(4-{[(2-Dimethylamino- ethyl)-ethyl-amino]-methyl}- phenyl)-furan-2-carboxylic acid hydroxyamide |
| L33 | 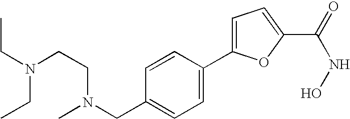 | 346 | 5-(4-{[(2-Diethylamino- ethyl)-methyl-amino]- methyl}-phenyl)-furan-2- carboxylic acid hydroxyamide |
| L34 | 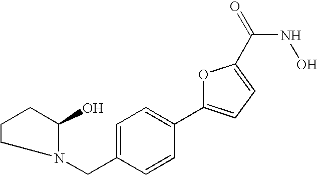 | 317 | 5-[4-(3-Hydroxy-piperidin-1- ylmethyl)-phenyl]-furan-2- carboxylic acid hydroxyamide |
| L35 | 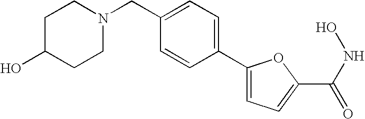 | 317 | 5-[4-(4-Hydroxy-piperidin-1- ylmethyl)-phenyl]-furan-2- carboxylic acid hydroxyamide |
| L36 | 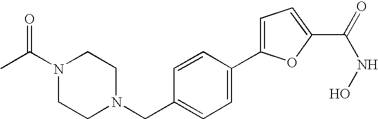 | 344 | 5-[4-(4-Acetyl-piperazin-1- ylmethyl)-phenyl]-furan-2- carboxylic acid hydroxyamide |
| L37 | 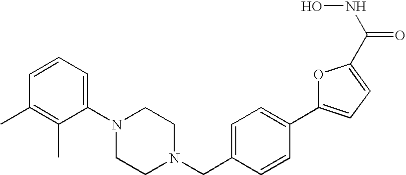 | 406 | 5-{4-[4-(2,3-Dimethyl- phenyl)-piperazin-1- ylmethyl]-phenyl}-furan-2- carboxylic acid hydroxyamide |
| L38 | 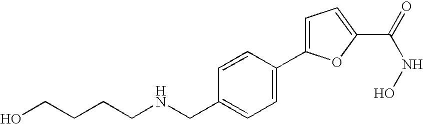 | 305 | 5-{4-[(4-Hydroxy- butylamino)-methyl]- phenyl}-furan-2-carboxylic acid hydroxyamide |
| L39 | 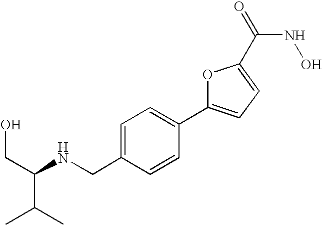 | 319 | (S)-5-{4-[(1-Hydroxymethyl- 2-methyl-propylamino)- methyl]-phenyl}-furan-2- carboxylic acid hydroxyamide |
| L40 | 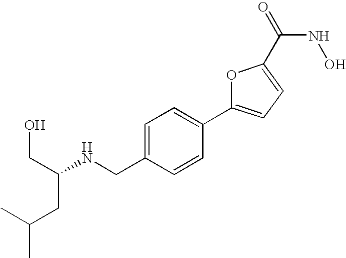 | 333 | (R)-5-{4-[(1-Hydroxymethyl- 3-methyl-butylamino)- methyl]-phenyl}-furan-2- carboxylic acid hydroxyamide |
| L41 | 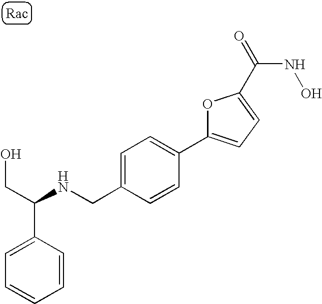 | 353 | 5-{4-[(2-Hydroxy-1-phenyl- ethylamino)-methyl]- phenyl}-furan-2-carboxylic acid hydroxyamide |
| L42 | 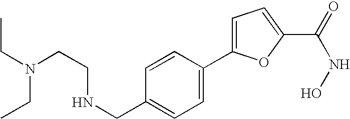 | 332 | 5-{4-[(2-Diethylamino- ethylamino)-methyl]- phenyl}-furan-2-carboxylic acid hydroxyamide |
| L43 | 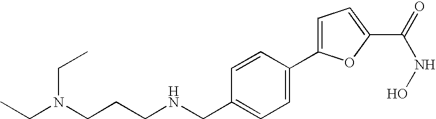 | 346 | 5-{4-[(3-Diethylamino- propylamino)-methyl]- phenyl}-furan-2-carboxylic acid hydroxyamide |
| L44 | 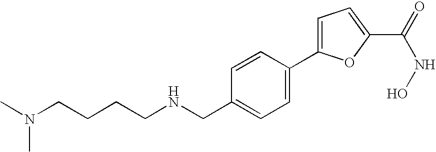 | 332 | 5-{4-[(4-Dimethylamino- butylamino)-methyl]- phenyl}-furan-2-carboxylic acid hydroxyamide |
| L45 | 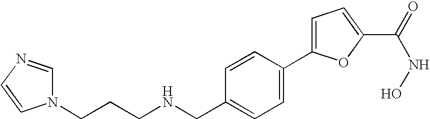 | 341 | 5-{4-[(3-Imidazol-1-yl- propylamino)-methyl]- phenyl}-furan-2-carboxylic acid hydroxyamide |

By methods analogous to those disclosed above [as described in Schemes (I to VI) and examples (1 to 46)] and by varying the starting materials used in the synthesis, a wide variety of compounds of Formula (I) could be prepared, including, but not limited to, those in Table 4.

TABLE 4

| No. | Structure |
|---|---|
| V1 | 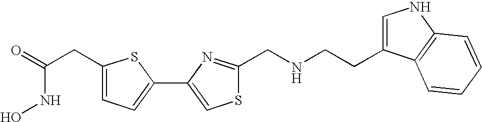 |
| V2 | 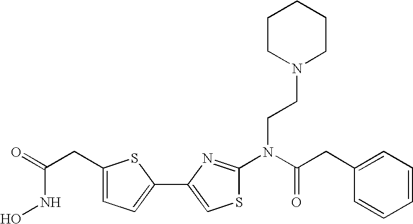 |
| V3 | 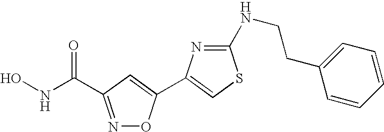 |
| V4 | 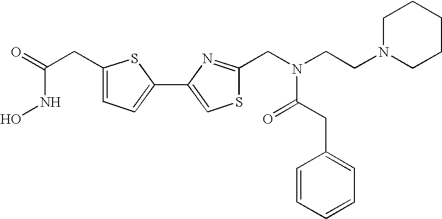 |
| V5 | 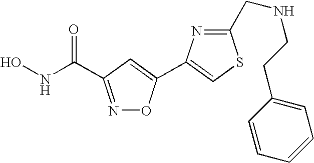 |
| V6 | 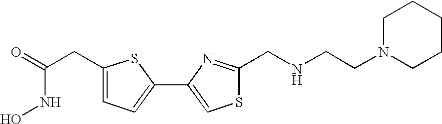 |
| V7 | 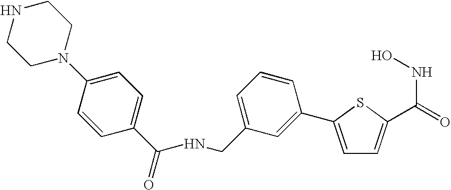 |
| V8 | 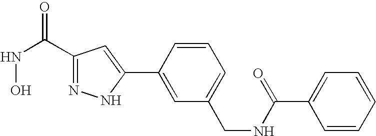 |
| V9 | 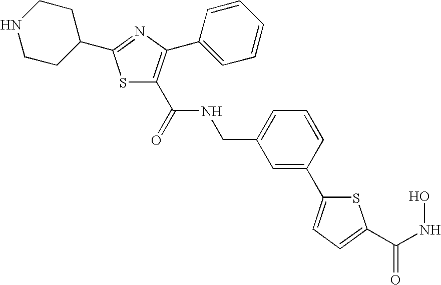 |
| V10 | 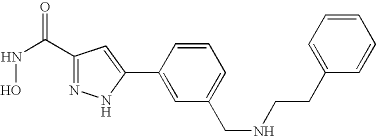 |
| V11 | 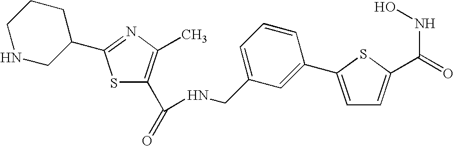 |
| V12 | 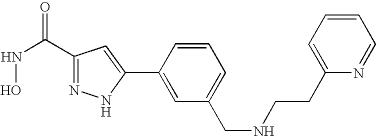 |
| V13 | 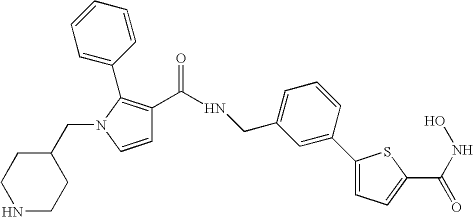 |
| V14 | 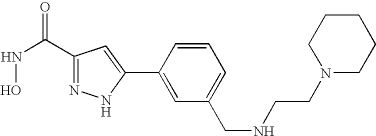 |
| V15 | 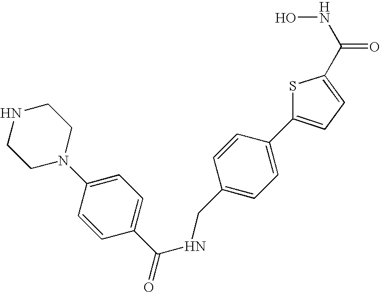 |
| V16 | 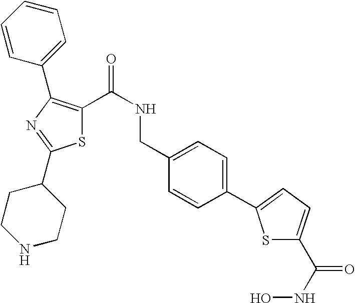 |
| V17 | 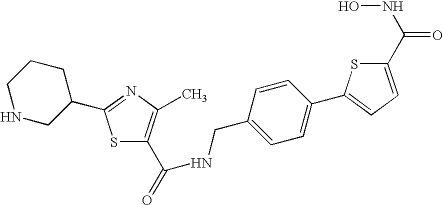 |
| V18 | 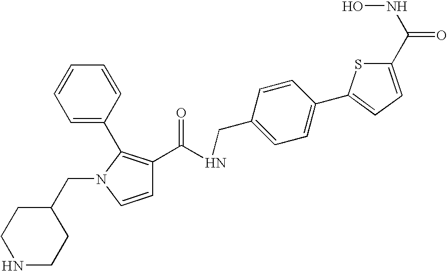 |
| V19 | 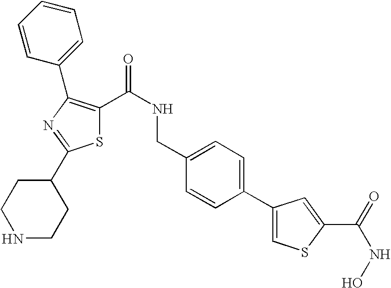 |
| V20 | 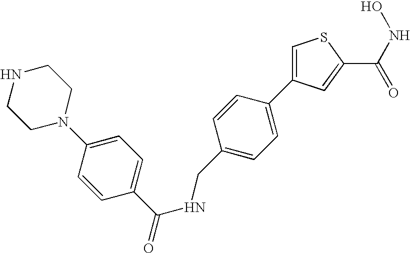 |
| V21 | 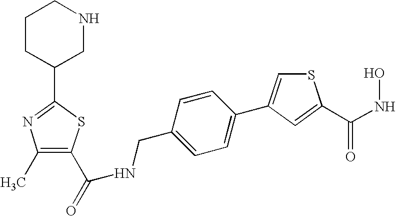 |
| V22 | 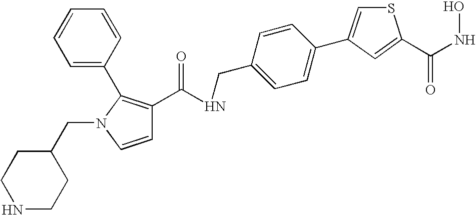 |
| V23 | 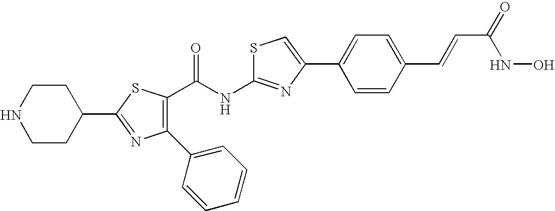 |
| V24 | 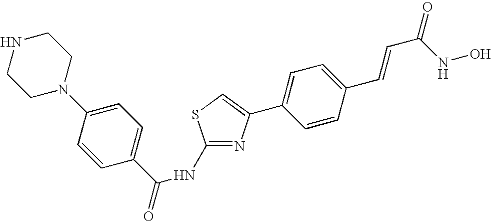 |
| V25 | 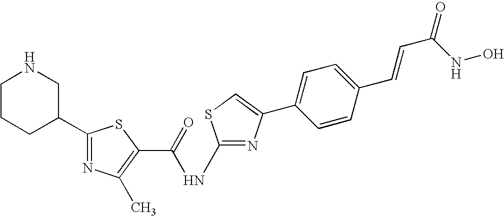 |
| V26 | 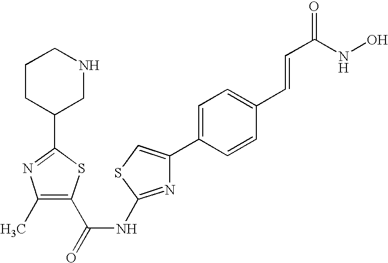 |
| V27 | 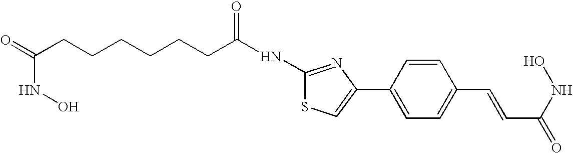 |
| V28 | 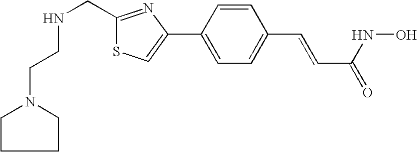 |
| V29 | 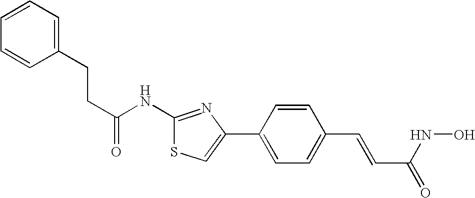 |
| V30 | 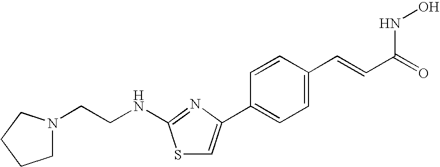 |
| V31 | 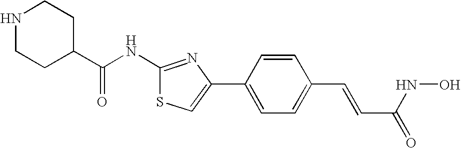 |
| V32 | 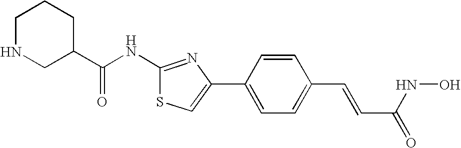 |
| V33 | 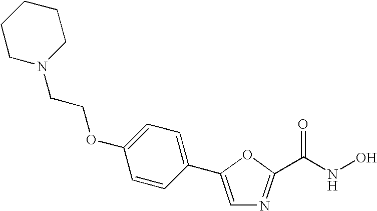 |
| V34 | 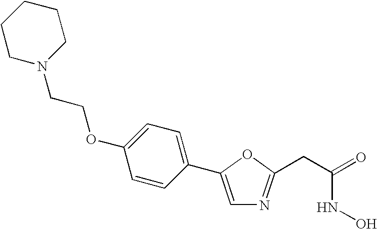 |
| V35 | 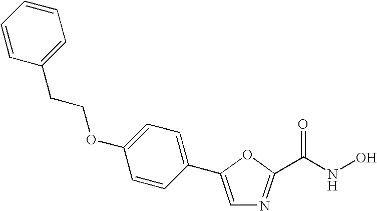 |
| V36 | 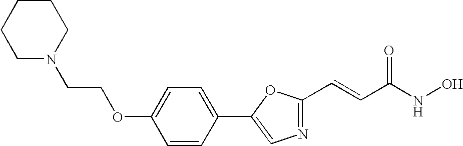 |
| V37 | 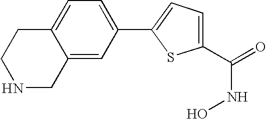 |
| V38 | 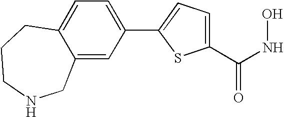 |
| V39 | 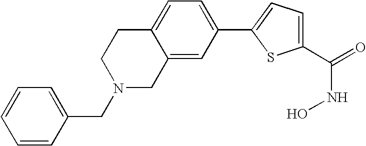 |
| V40 | 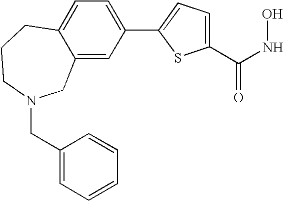 |
| V41 | 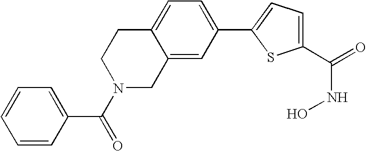 |
| V42 | 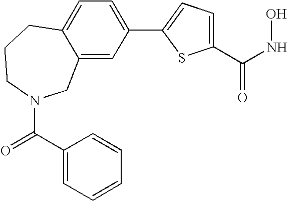 |
| V43 | 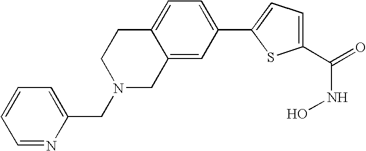 |
| V44 | 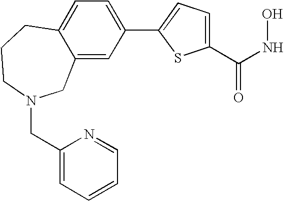 |
| V45 | 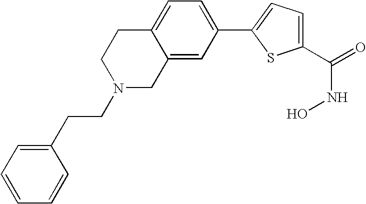 |
| V46 | 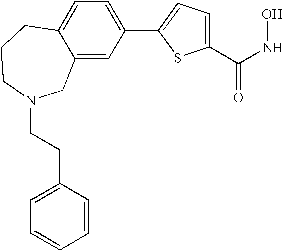 |
| V47 | 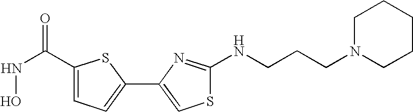 |
| V48 | 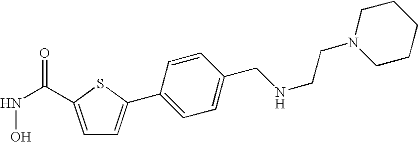 |
| V49 | 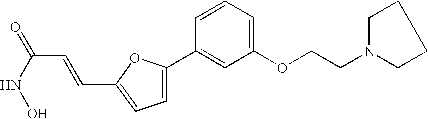 |
| V50 | 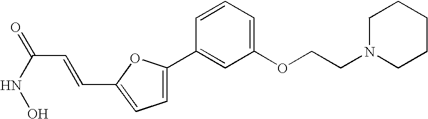 |
| V51 | 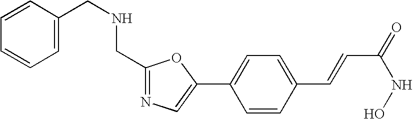 |
| V52 | 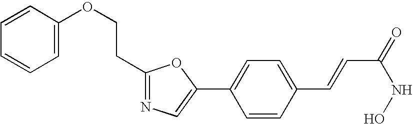 |
| V53 | 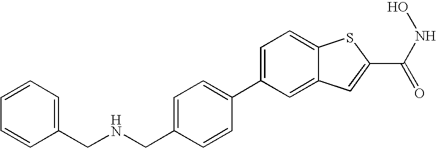 |
| V54 | 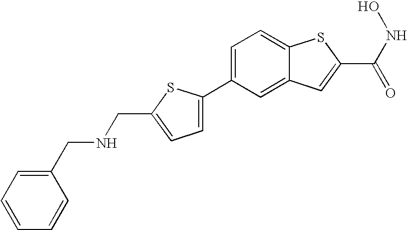 |
| V55 | 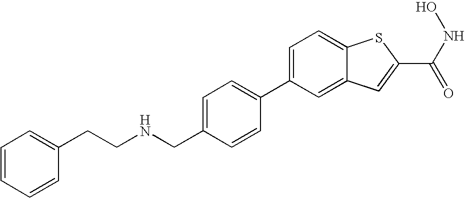 |
| V56 | 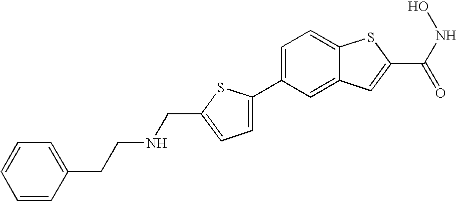 |
| V57 | 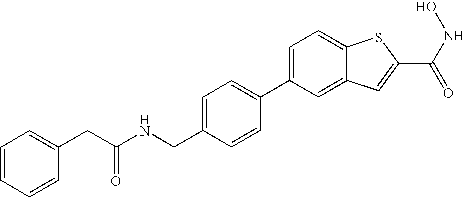 |
| V58 | 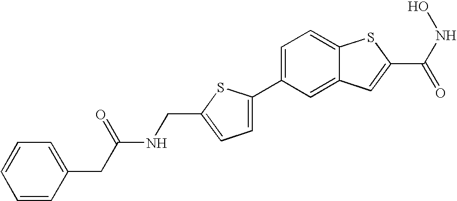 |
| V59 | 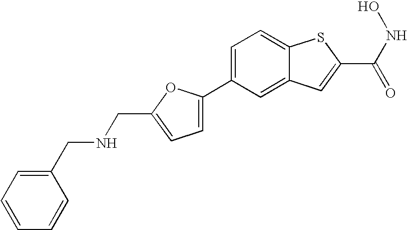 |
| V60 | 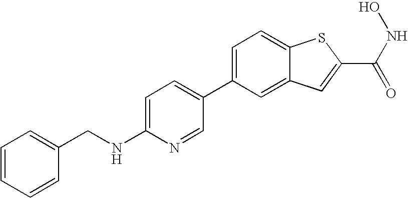 |
| V61 | 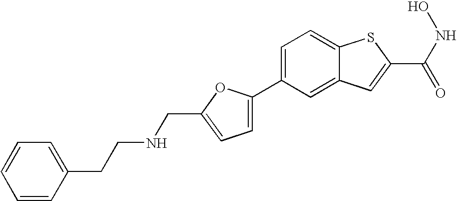 |
| V62 | 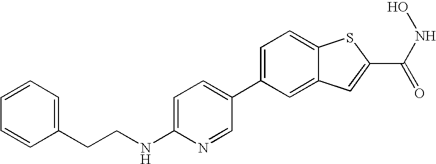 |
| V63 | 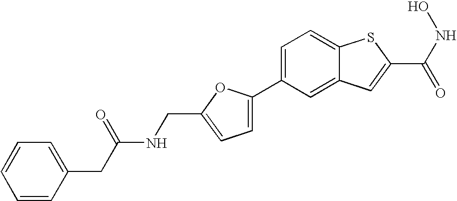 |
| V64 | 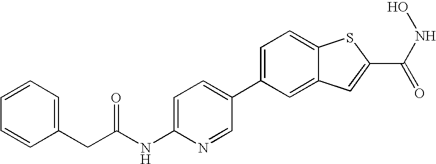 |
| V65 | 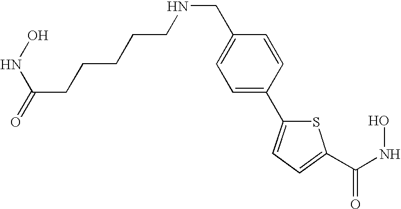 |
| V66 | 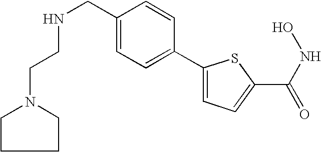 |
| V67 | 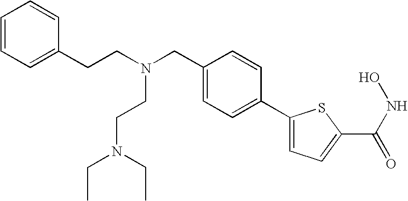 |
| V68 | 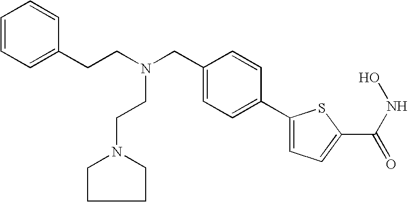 |
| V69 | 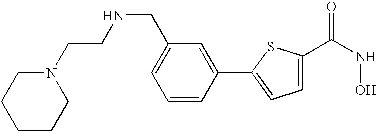 |
| V70 | 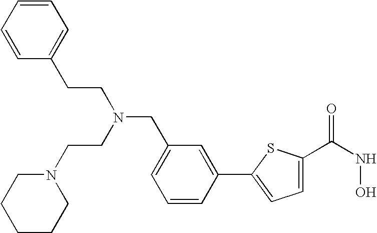 |
| V71 | 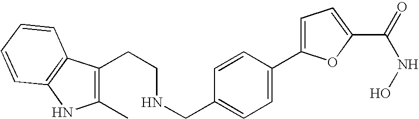 |
| V72 | 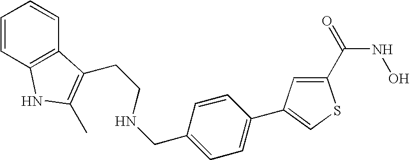 |
| V73 | 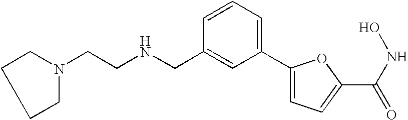 |
| V74 | 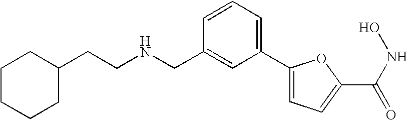 |
| V75 | 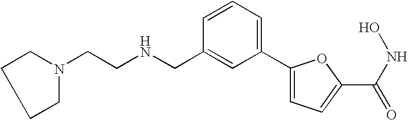 |
| V76 | 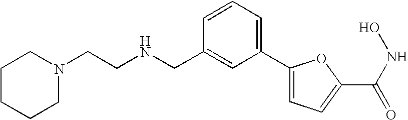 |
| V77 | 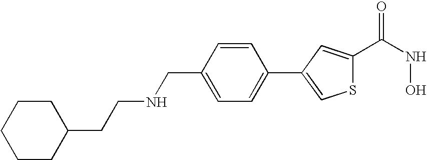 |
| V78 | 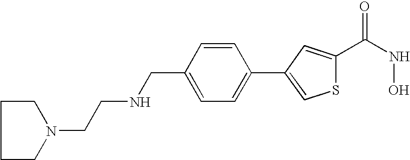 |

### Biological Testing and Enzyme Assays

Recombinant GST-HDAC1 and GST-HDAC-8 Protein expression and purification

Human cDNA library was prepared using cultured SW620 cells. Amplification of human HDAC1 and HDAC8 coding region from this cDNA library was cloned separately into the baculovirus expression pDEST20 vector and pFASTBAC vector respectively (GATEWAY Cloning Technology, Invitrogen Pte Ltd). The pDEST20-HDAC1 and pFASTBAC-HTGST-HDAC8 constructs were confirmed by DNA sequencing. Recombinant baculovirus was prepared using the Bac-To-Bac method following the manufacturer's instruction (Invitrogen Pte Ltd). Baculovirus titer was determined by plaque assay to be about 10⁸ PFU/ml.

Expression of GST-HDAC1 or HTGST-HDAC8 was done by infecting SF9 cells (Invitrogen Pte Ltd) with pDEST20-HDAC1 or pFASTBAC-GST-HDAC8 baculovirus at MOI=1 for 48 h. Soluble cell lysate was incubated with pre-equilibrated Glutathione Sepharose 4B beads (Amersham) at 4° C. for 2 h. The beads were washed with PBS buffer for 3 times. The GST-HDAC1 protein or GST-HDAC8 protein was eluted by elution buffer containing 50 mM Tris, pH8.0, 150 mM NaCl, 1% Triton X-100 and 10 mM or 20 mM reduced Glutathione. The purified GST-HDAC1 protein or purified GST-HDAC8 protein was dialyzed with HDAC storage buffer containing 10 mM Tris, pH7.5, 100 mM NaCl and 3 mM MgCl₂. 20% Glycerol was added to purified GST-HDAC1 protein or purified GST-HDAC8 before storage at −80° C.

In vitro HDAC assay for determination of IC₅₀ values

The assay has been carried out in 96 well format and the BIOMOL fluorescent-based HDAC activity assay has been applied. The reaction composed of assay buffer, containing 25 mM Tris pH 7.5, 137 mM NaCl, 2.7 mM KCl, 1 mM MgCl₂, 1 mg/ml BSA, tested compounds, 500 nM HDAC8 enzyme or 600 nM HDAC1 enzyme, 200 μM Flur de lys p53 peptide substrate for HDAC8 enzyme or 500 μM Flur de lys generic substrate for HDAC1 enzyme and subsequently was incubated at room temperature for 2 h. Flur de lys Developer was added and the reaction was incubated for 10 min. Briefly, deacetylation of the substrate sensitizes it to the developer, which then generates a fluorophore The fluorophore is excited with 360 nm light and the emitted light (460 nm) is detected on a fluorometric plate reader (Tecan Ultra Microplate detection system, Tecan Group Ltd.).

The analytical software, Prism 3.0® (GraphPad Software Inc) has been used to generate IC₅₀ from a series of data. The HDAC enzyme inhibition results of representative compounds are shown in Table 5.

TABLE 5
HDAC enzyme inhibition activities of representative examples

| | HDAC1 Enzyme | HDAC8 Enzyme |
|---|---|---|
| Compound | Activity, IC₅₀ (μM) | Activity, IC₅₀ (μM) |
| Example 1 | >100 | 0.041 |
| Example 6 | 2.78 | 0.040 |
| Example 8 | 2.76 | 0.089 |
| Example 10 | >100 | 0.14 |
| Example 15 | 1.13 | 0.29 |
| Example 16 | 0.70 | 0.038 |
| Example 17 | 1.40 | 0.34 |
| Example 18 | >100 | 0.35 |
| Example 23 | 0.51 | 0.10 |
| Example 26 | 0.066 | 0.016 |
| Example 27 | 0.20 | 0.12 |
| Example 28 | 0.015 | 0.014 |
| Example 29 | 0.087 | 0.026 |
| Example 30 | 0.22 | 0.050 |
| Example 31 | 0.017 | 0.008 |
| Example 44 | 1.42 | 0.11 |

Cell-based Proliferation assay for determination of GI₅₀ values

Three different cancer cell lines were obtained from ATCC: Human colon cancer cell line (Colo205), human breast cancer cell lines (MDA-MB435), and human lung cancer cell line (NCl-H522). Colo205 cells and NCl-H522 were cultivated in RPMI 1640 containing 2 mM

L-Glutamine, 5% FBS, 1.0 mM Na Pyruvate. MDA-MB435 cells were cultivated in DMEM containing 2 mM L-Glutamine, 5% FBS. Colo205 cells were seeded in 96-wells plate at 2000 and 5000 cells per well respectively. MDA-MB435 and NCl-H522 cells were seeded in 96-wells plate at 6000 cells per well. The plates were incubated at 37° C., 5% CO₂, for 24 h. Cells were treated with compounds at various concentrations for 96 h. Cell growth was then monitored using cyquant cell proliferation assay (Invitrogen Pte Ltd). Dose response curves were plotted to determine GI₅₀ values for the compounds using XL-fit (ID Business Solution, Emeryville, Calif.).

The cellular or growth inhibition activity results of representative compounds are shown in Table 6. These data indicate that compounds in this invention are active in inhibition of tumor cell growth. In addition, representative compounds have also demonstrated their ability to inhibit growth in other types of cancer cell lines including lung cancer cell lines (e.g. A549), prostate cancer cell line (e.g. PC3), leukemia cell line (e.g. HL-60), lymphoma cell line (e.g. Ramos) and pancreatic cancer cell line (MIAPaCA2) (data not shown).

TABLE 6
Cellular activities (GI₅₀, μM) of representative examples

| Compound | NCI H552 | MDA-MB435 | Colo205 |
|---|---|---|---|
| Example 1 | 2.36 | | 12.07 |
| Example 9 | 3.01 | | 5.47 |
| Example 23 | 13.30 | 4.46 | 7.72 |
| Example 26 | | 2.66 | 1.86 |
| Example 27 | | 1.49 | 2.39 |

Histone H3, H4 and H2A acetylation assay

A hallmark of histone deacetylase (HDAC) inhibition is the increase in the acetylation level of histones. Histone acetylation, including H3, H4 and H2A can be detected by immuno-blotting (western-blot). Colo205 cells, approximately 1.5×10⁶ cells/10 cm dish, were seeded in the previously described medium, cultivated for 24 h and subsequently treated with HDAC inhibitory agents at 0.1, 1, 5 and 10 μM final concentration. After 24 h, cells were harvested and lysed according to the instruction from Sigma Mammalian Cell Lysis Kit. The protein concentration was quantified using BCA method (Sigma Pte Ltd). The protein lysate was separated using 4-12% bis-tris SDS-PAGE gel (Invitrogen Pte Ltd) and was transferred onto PVDF membrane (BioRad Pte Ltd). The membrane was probed separately using primary antibody specific for acetylated H3, acetylated H4 or acetylated H2A (Upstate Pte Ltd). The detection antibody, goat anti rabbit antibody conjugated with horse radish peroxidase (HRP) was used according to the manufacturing instruction (Pierce Pte Ltd). After removing the detection antibody from the membrane, an enhanced chemiluminescent substrate for detection of HRP (Pierce Pte Ltd) was added onto the membrane. After removing the substrate, the membrane was exposed to an X-ray film (Kodak) for 1 sec-20 mins. The X-ray film was developed using the X-ray film processor. The density of each band observed on the developed film could be analysed using UVP Bioimaging software (UVP, Inc, Upland, Calif.). The values were then normalized against the density of actin in the corresponding samples to obtain the expression of the protein.

The results of histone deacetylase assay are shown in Table 7.

TABLE 7
Effect of representative examples on accumulation of acetylated histone

| Compound | Histone 3 acetylation | Histone 4 acetylation |
|---|---|---|
| Example 1 | Active (after 48 hrs) | |
| Example 23 | Active | Active |
| Example 26 | Active | Active |
| Example 27 | Active | Active |
| Example 30 | Active | Active |

“Active” means accumulation of acetylated histone was observed when compared with control (without compound).

These data demonstrate that compounds in this invention inhibit histone deacetylases, thereby resulting in accumulation of acetylated histones.

Apoptosis Assays

In various therapies such as for proliferative disorders like cancer, the selective induction of apoptosis in proliferating cells such as tumor cells is one of the desirable approaches, and can be mediated by treatment with various anti-proliferative compounds [Blagosklonny MV, Oncogene, 23(16): 2967 (2004); Kaufmann and Eamshaw, Exp Cell Res. 256(1): 42-9 (2000)]. Programmed cell death or apoptosis is the cellular response to stress factors such as DNA damage introduced during conventional anti-cancer treatment. The concerted sequence of events during apoptosis, clearly differentiate this pathway from a non-coordinated form of cell death called necrosis. During the course of apoptosis, characteristic phenotypical cellular changes occur, which include the condensation of chromatin, the shrinkage of cells and finally the fragmentation of chromosomal DNA. One of the very early change caused by apoptotic events occurs in the phospholipids bilayer of the plasma membrane. The phospholipid phosphatidylserine is translocated from the inner to the outer side of the plasma-membrane and, as a result, is exposed to the extracellular space. One way of detecting early apoptotic cells is to determine the amount of phosphatidyl-serine at the extracellular side of the plasma-membrane which is accomplished by the standard flow cytometric method of Annexin V staining. The phospholipids recognizing protein Annexin V binds with high affinity to these inverted and exposed phosphatidyl-serines.

The ability of the compounds in this invention to induce apoptosis was tested in Ramos Burkitt-lymphoma cells. This cell line is one of the gold standard cell lines commonly used as a tissue culture model for B cell lymphoma. Representative compounds as indicated below were added to 80,000 cells per 500 μl growth medium (RPMI1640 medium supplemented with 2 mM L-Glutamine, 10% heat-inactivated FBS, 1 mM Na-Pyruvate and 10 mM HEPES) in 24 well format at various concentrations. Two days after the start of treatment, cells were collected and subjected to the Annexin V staining protocol following the instructions of the manufacturer (BD Biosciences). By using propidium iodide (PI) as a viability control, cells that stain positive for Annexin V, but negative for PI, are undergoing apoptosis. The percentage of cells in late apoptosis after treatment as shown in Table 8 was derived from a standard flow cytometry (FACS) analysis [Steensma et al, Methods Mol Med 85:323-32 (2003]. Table 8 below shows the percentage of late apoptotic cells 48 h after treatment with 10 μM of the representative compounds of this invention.

TABLE 8
Apoptosis induced in a cancer cell line by representative examples

| | % cells in late |
|---|---|
| Compound | apoptosis (Ramos) |
| Example 1 | 76 |
| Example 8 | 74 |
| Example 16 | 90 |
| Example 44 | 82 |
| 3′-(Phenylacetylamino-methyl)-biphenyl-4-carboxylic acid | 89 |
| hydroxyamide | |
| 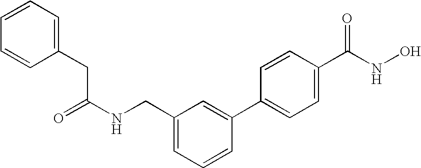 | |

In addition, selected compounds are tested for their ability to induce apoptosis in HL-60 cells which is an acute promyelocytic leukemia cell line (data not shown). As the results shown above indicate, the compounds disclosed in this invention can be used to treat cancers including hematologic malignancies (e.g. lymphoma and leukemia).

In vivo Xenograft Tumor Study

In data not shown, selected compounds were tested for maximal tolerated dose in normal mice and were found to be well tolerated by the mice with no obvious signs of toxicity or side effects in the dose range applied (which can be >100 mg/kg/day).

The efficacy of the compounds of the invention can then be determined using in vivo animal xenograft studies. The animal xenograft model is one of the most commonly used in vivo cancer rnodels.

In these studies Female athymic nude mice (Harlan), 12-14 weeks of age would be implanted subcutaneously in the flank with 5×10⁶ cells of HCT116 or with 1×10⁶ cells of Colo205 human colon carcinoma suspended in 50% Matrigel. When the tumor reaches the size 100 mm³, the xenograft nude mice would be paired-match into various treatment groups. The selected HDAC inhibitors would be dissolved in appropriate vehicles, such as 10% DMA/10% Cremophore/80% water and administered to xenograft nude mice intraperitoneally by daily for 14 days. The dosing volume will be 0.2-ml/20 g mouse. Paclitaxol, used as positive control, will be prepared for intravenous administration in 10% Ethanol/10% Cremophore/80% water. The dosing volume for Paclitaxol will be 0.015-ml/g mouse. Tumor volume will be calculated every second day of post injection using the formula: Tumor volume (mm³)=(w²×l)/2, where w=width and l=length in mm of an HCT116 or Colo205 carcinoma [Beverly AT, in Tumor Models in Cancer Research, published by Humana Press, New Jersey, 593-612, 2002]. Compounds in this invention that are tested would show significant reduction in tumor volume relative to controls treated with vehicle only. The activity of histone deacetylase when measured shall be reduced and results in accumulation of acetylated histone relative to vehicle treated control group. The result will therefore indicate that compounds in this invention are efficacious in treating a proliferative disorder such as cancer.

The details of specific embodiments described in this inventlon are not to be construed as limitations. Various equivalents and modifications may be made without departing from the essence and scope of this invention, and it is understood that such equivalent embodiments are part of this invention.

1. A compound of the Formula (I) 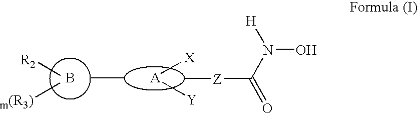
wherein
Z is a single bond or a C₁-C₄ hydrocarbon chain containing no more than 1 double or triple bond, optionally substituted with one or more substituents independently selected from the group consisting of C₁-C₄ alkyl;
A is an aromatic ring selected from the group consisting of optionally substituted arylene and optionally substituted heteroarylene, wherein A is not benzimidazole and when Z is a single bond then A is not selected from the group consisting of phenylene and six-membered heteroarylene containing 3 or less than 3 nitrogens;
B is an aromatic ring selected from the group consisting of aryl and heteroaryl and wherein A and B can not both be phenylene and wherein when Z is a single bond then B is not a bicyclic aryl or bicyclic heteroaryl;
wherein A and B are connected via a carbon-carbon bond;
R₂ is selected from the group consisting of halogen, alkyl, alkenyl, alkynyl, haloalkyl, haloalkenyl, heteroalkyl, cycloalkyl, cycloalkenyl, heterocycloalkyl, heterocycloalkenyl, aryl, heteroaryl, cycloalkylalkyl, heterocycloalkylalkyl, arylalkyl, heteroarylalkyl, arylalkenyl, cycloalkylheteroalkyl, heterocycloalkylheteroalkyl, heteroarylheteroalkyl, arylheteroalkyl, hydroxy, hydroxyalkyl, alkoxy, alkoxyalkyl, alkoxyaryl, alkenyloxy, alkynyloxy, cycloalkylkoxy, heterocycloalkyloxy, aryloxy, heteroaryloxy, arylalkyloxy, amino, alkylamino, aminoalkyl, acylamino, arylamino, phenoxy, benzyloxy, COOH, COOR₄, SH, CONHR₄, NHR₄, —(CH₂)ₙNHCOR₄, NHCOR₄, NHCOOR₄ NHCONHR₄, C(═NOH)R₄, NHSOR₄ NHSO₂R₄, —(CH₂)_{n—NR}₆R₇, alkoxycarbonyl, alkylaminocarbonyl, sulfonyl, alkylsulfonyl, alkylsulfinyl, aryisulfonyl, arylsulfinyl, aminosulfonyl, aminosulfinyl, SR₄ and acyl each of which may optionally be substituted, provided that R₂ does not contain the moiety NHCONHCO or NHCONHSO₂;
R₃ is selected from the group consisting of H, halogen, alkyl, alkenyl, alkynyl, haloalkyl, haloalkenyl, heteroalkyl, cycloalkyl, cycloalkenyl, heterocycloalkyl, heterocycloalkenyl, aryl, heteroaryl, cycloalkylalkyl, heterocycloalkylalkyl, arylalkyl, heteroarylalkyl, arylalkenyl, cycloalkylheteroalkyl, heterocycloalkylheteroalkyl, heteroarylheteroalkyl, arylheteroalkyl, hydroxy, hydroxyalkyl, alkoxy, alkoxyalkyl, alkoxyaryl, alkenyloxy, alkynyloxy, cycloalkylkoxy, heterocycloalkyloxy, aryloxy, heteroaryloxy, arylalkyloxy, amino, alkylamino, aminoalkyl, acylamino, arylamino, phenoxy, benzyloxy, COOH, COOR₄, SH, CONHR₄, NHR₄, —(CH₂)ₙNHCOR₄, NHCOR₄, NHCOOR₄ NHCONHR₄, C(═NOH)R₄, NHSOR₄ NHSO₂R₄, —(CH₂)ₙ—NR₆R₇, alkoxycarbonyl, alkylaminocarbonyl, sulfonyl, alkylsulfonyl, alkylsulfinyl, arylsulfonyl, arylsulfinyl, aminosulfonyl, aminosulfinyl, SR₄ and acyl; each of which may optionally be substituted provided that R₃ does not contain the moiety NHCONHCO or NHCONHSO₂;
or R₂ and R₃ together with portion of ring B may form a non-aromatic ring fused to B;
X and Y are the same or different and are independently selected from the group consisting of H, halogen, —CN, —NO₂, —CF₃, —OCF₃, alkyl, alkenyl, alkynyl, haloalkyl, haloalkenyl, haloalkynyl, heteroalkyl, cycloalkyl, cycloalkenyl, heterocycloalkyl, heterocycloalkenyl, heteroaryl, hydroxy, hydroxyalkyl, alkoxy, alkoxyalkyl, alkoxyaryl, alkoxyheteroaryl, alkenyloxy, alkynyloxy, cycloalkyloxy, cycloalkenyloxy, heterocycloalkyloxy, heterocycloalkenyloxy, aryloxy, heteroaryloxy, arylalkyl, heteroarylalkyl, arylalkyloxy, amino, alkylamino, acylamino, aminoalkyl, arylamino, sulfonylamino, sulfinylamino, sulfonyl, alkylsulfonyl, arylsulfonyl, aminosulfonyl, aminoalkyl, alkoxyalky, —COOH, —C(O)OR₄, —COR₄, —SH, —SR₄, —OR₄, acyl and —NR₈R₉ each of which may be optionally substituted;
each R₄ is independently selected from the group consisting of H, alkyl, alkenyl, alkynyl, haloalkyl, heteroalkyl, cycloalkyl, heterocycloalkyl, aryl, heteroaryl, cycloalkylalkyl, heterocycloalkylalkyl, arylalkyl, heteroarylalkyl and acyl each of which may be optionally substituted;
each R₆ and R₇ is independently selected from the group consisting of H, alkyl, alkenyl, alkynyl, haloalkyl, heteroalkyl, cycloalkyl, heterocycloalkyl, aryl, heteroaryl, cycloalkylalkyl, heterocycloalkylalkyl, arylalkyl, heteroarylalkyl and acyl; each of which may be optionally substituted;
each R₈ and R₉ is independently selected from the group consisting of H, alkyl, alkenyl, alkynyl, haloalkyl, heteroalkyl, cycloalkyl, heterocycloalkyl, aryl, heteroaryl, cycloalkylalkyl, heterocycloalkylalkyl, arylalkyl, heteroarylalkyl and acyl; each of which may be optionally substituted;
n is an integer from 0 to 6,
m is an integer from 0 to 4;
or a pharmaceutically acceptable salt or prodrug thereof,
wherein when A is 2,5-oxazolene and Z is single bond, R₂=R₃=H, then B is not a phenyl, 4-Cl-phenyl, 4-CH₃—O-phenyl or 4-NO₂-phenyl.

2. A compound according to claim 1 having the Formula (Ia) 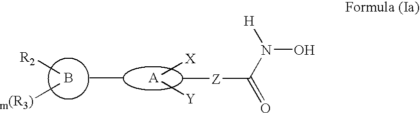
wherein
Z is a single bond or a C₁-C₄ hydrocarbon chain which may contain 0 to 1 double or triple bonds, unsubstituted or substituted with one or more substituents independently selected from the group consisting of C₁-C₄ alkyl;
A is an aromatic ring selected from the group consisting of optionally substituted arylene and optionally substituted heteroarylene, wherein A is not benzimidazole and when Z is a single bond then A is not selected from the group consisting of phenylene and six-membered heteroarylene containing 3 or less than 3 nitrogens;
B is an aromatic ring selected from the group consisting of aryl and heteroaryl and wherein A and B can not both be phenylene and wherein when Z is a single bond then B is not a bicyclic aryl or bicyclic heteroaryl;
wherein A and B are connected via a carbon-carbon bond;
R₂ is selected from C₁-C₁₀ alkyl, alkenyl, heteroalkyl, haloalkyl, alkynyl, aryl, cycloalkyl, heterocycloalkyl, heteroaryl, C₄-C₉ heterocycloalkylalkyl, cycloalkylalkyl (e.g., cyclopropylmethyl), arylalkyl (e.g. benzyl), heteroarylalkyl (e.g. pyridylmethyl), hydroxyl, hydroxyalkyl, alkoxy, amino, alkylamino, aminoalkyl, acylamino, phenoxy, alkoxyalkyl, benzyloxy, alkylsulfonyl, arylsulfonyl, aminosulfonyl, —C(O)OR₄, —C(O)OH, —SH, —CONHR₄, —NHCONHR₄, C(═NOH)R₄, —C(O)C(O)OR₄, C(O)CONHR₄, CON(R₅)OR₄, COCON(R₄)OR₄, NHCOR₄, and acyl; each of the above is unsubstituted or optionally substituted with one or more substituents independently selected from the group consisting of: halogen; ═O; ═S; —CN; and —NO₂; and alkyl, alkenyl, heteroalkyl, haloalkyl, alkynyl, aryl, cycloalkyl, heterocycloalkyl, heteroaryl, hydroxyl, hydroxyalkyl, alkoxy, alkylamino, aminoalkyl, acylamino, phenoxy, alkoxyalkyl, benzyloxy, alkylsulfonyl, arylsulfonyl, aminosulfonyl, —C(O)OR₅, —C(O)OH, —SH, —C(O)C(O)OR₅, C(O)CONHR₅, CON(R₅)OR₅, COCON(R₅)OR₅, NHCOR₅, and acyl; wherein R₂ does not contain the moiety NHCONHCO or NHCONHSO₂;
R₃ is selected from H, C₁-C₁₀ alkyl, alkenyl, heteroalkyl, haloalkyl, alkynyl, aryl, cycloalkyl, heterocycloalkyl, heteroaryl, C₄-C₉ heterocycloalkylalkyl, cycloalkylalkyl (e.g., cyclopropylmethyl), arylalkyl (e.g. benzyl), heteroarylalkyl (e.g. pyridylmethyl), hydroxyl, hydroxyalkyl, alkoxy, amino, alkylamino, aminoalkyl, acylamino, phenoxy, alkoxyalkyl, benzyloxy, alkylsulfonyl, arylsulfonyl, aminosulfonyl, —C(O)OR₄, —C(O)OH, —SH, —CONHR₄, —NHCONHR₄, C(═NOH)R₄, —C(O)C(O)OR₄, C(O)CONHR₄, CON(R₅)OR₄, COCON(R₄)OR₄, NHCOR₄, and acyl; each of the above is unsubstituted or optionally substituted with one or more substituents independently selected from the group consisting of: halogen; ═O; ═S; —CN; and —NO₂; and alkyl, alkenyl, heteroalkyl, haloalkyl, alkynyl, aryl, cycloalkyl, heterocycloalkyl, heteroaryl, hydroxyl, hydroxyalkyl, alkoxy, alkylamino, aminoalkyl, acylamino, phenoxy, alkoxyalkyl, benzyloxy, alkylsulfonyl, arylsulfonyl, aminosulfonyl, —C(O)OR₅, —C(O)OH, —SH, —C(O)C(O)OR₅, C(O)CONHR₅, CON(R₅)OR₅, COCON(R₆)OR₅, NHCOR₅, and acyl; wherein R₃ does not contain the moiety NHCONHCO or NHCONHSO₂;
or R₂ and R₃ together with portion of ring B may form a non-aromatic ring fused to B;
X and Y are the same or different and independently selected from the group consisting of: H, halo, C₁-C₄ alkyl, such as CH₃ and CF₃, NO₂, OR₄, SR₄, C(O)R₅, CN, and NR₈ R₉;
R₄ is selected from H, C₁-C₄ alkyl, heteroalkyl, aryl, heteroaryl, acyl;
R₅ is selected from H, C₁-C₄ alkyl;
R₈ and R₉ are the same or different and independently selected from the group consisting of H, C₁-C₆ alkyl, C₄-C₉ cycloalkyl, C₄-C₉ heterocycloalkyl, aryl, heteroaryl, arylalkyl, and heteroarylalkyl;
m is an integer from 0 to 4;
or a pharmaceutically acceptable salt or prodrug thereof,
wherein when A is 2,5oxazolene and Z is single bond, R₂=R₃=H, then B is not a phenyl, 4-Cl-phenyl, 4-CH₃—O-phenyl or 4-NO₂₋phenyl.

3. A compound according to claim 1 having the Formula (Ib) 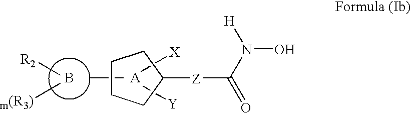
wherein
Z is a single bond or a C₁-C4 hydrocarbon chain which may contain 0 to 1 double bond or triple bond, unsubstituted or substituted with one or more substituents independently selected from the group consisting of C₁-C₄ alkyl;
A is an optionally substituted five-membered heteroarylene;
B is an aromatic ring which is selected from the group consisting of aryl and heteroaryl; wherein when Z is a single bond then B is not a bicyclic aryl or bicyclic heteroaryl;
wherein A and B are connected via a carbon-carbon bond;
R₂ is selected from the group consisting of halogen, alkyl, alkenyl, alkynyl, haloalkyl, haloalkenyl, heteroalkyl, cycloalkyl, cycloalkenyl, heterocycloalkyl, heterocycloalkenyl, aryl, heteroaryl, cycloalkylalkyl, heterocycloalkylalkyl, arylalkyl, heteroarylalkyl, arylalkenyl, cycloalkylheteroalkyl, heterooycloalkylheteroalkyl, heteroarylheteroalkyl, arylheteroalkyl, hydroxy, hydroxyalkyl, alkoxy, alkoxyalkyl, alkoxyaryl, alkenyloxy, alkynyloxy, cycloalkylkoxy, heterocycloalkyloxy, aryloxy, heteroaryloxy, arylalkyloxy, amino, alkylamino, aminoalkyl, acylamino, arylamino, phenoxy, benzyloxy, COOH, COOR₄, SH, CONHR₄, NHR₄, —(CH₂)ₙNHCOR₄, NHCOR₄, NHCOOR₄ NHCONHR₄, C(═NOH)R₄, NHSOR₄ NHSO₂R₄, —(CH₂)ₙ—NR₈R₇, alkoxycarbonyl, alkylaminocarbonyl, sulfonyl, alkylsulfonyl, alkylsulfinyl, arylsulfonyl, arylsulfinyl, aminosulfonyl, aminosulfinyl, SR₄ and acyl; each of which may optionally be substituted, wherein R₂ does not contain the moiety NHCONHCO or NHCONHSO₂;
R₃ is selected from the group consisting of H, halogen, alkyl, alkenyl, alkynyl, haloalkyl, haloalkenyl, heteroalkyl, cycloalkyl, cycloalkenyl, heterocycloalkyl, heterocycloalkenyl, aryl, heteroaryl, cycloalkylalkyl, heterocycloalkylalkyl, arylalkyl, heteroarylalkyl, arylalkenyl, cycloalkylheteroalkyl, heterocycloalkylheteroalkyl, heteroarylheteroalkyl, arylheteroalkyl, hydroxy, hydroxyalkyl, alkoxy, alkoxyalkyl, alkoxyaryl, alkenyloxy, alkynyloxy, cycloalkylkoxy, heterocycloalkyloxy, aryloxy, heteroaryloxy, arylalkyloxy, amino, alkylamino, aminoalkyl, acylamino, arylamino, phenoxy, benzyloxy, COOH, COOR₄, SH, CONHR₄, NHR₄, —(CH₂)ₙNHCOR₄, NHCOR₄, NHCOOR₄ NHCONHR₄, C(═NOH)R₄, NHSOR₄ NHSO₂R₄, —(CH₂)ₙNRNR₇, alkoxycarbonyl, alkylaminocarbonyl, sulfonyl, alkylsulfonyl, alkylsulfinyl, arylsulfonyl, arylsulfinyl, aminosulfonyl, aminosulfinyl, SR₄ and acyl; each of which may optionally be substituted wherein R₃ does not contain the moiety NHCONHCO or NHCONHSO₂;
or R₂ and R₃ together with portion of ring B may form a non-aromatic ring fused to B;
X and Y are the same or different and are independently selected from the group consisting of H, halo, C₁-C₄ alkyl, such as CH₃ and CF₃, NO₂, OR₄, SR₄, C(O)R₅, CN, and NR₈ R₉;
R₄ is selected from H, C₁-C₄ alkyl, heteroalkyl, aryl, heteroaryl, acyl;
R₅ is selected from H, C₁-C₄ alkyl;
each R₆ and R₇ is independently selected from the group consisting of H, alkyl, alkenyl, alkynyl, haloalkyl, heteroalkyl, cycloalkyl, heterocycloalkyl, aryl, heteroaryl, cycloalkylalkyl, heterocycloalkylalkyl, arylalkyl, heteroarylalkyl and acyl each of which may be optionally substituted;
R₈ and R₉ are the same or different and are independently selected from the group consisting of H, C₁-C₆ alkyl, C₄-C₉ cycloalkyl, C₄-C₉ heterocycloalkyl, aryl, heteroaryl, arylalkyl, heteroarylalkyl;
n is an integer from 0 to 6;
m is an integer from 0 to 4;
or a pharmaceutically acceptable salt or prodrug thereof,
wherein when A is 2,5-oxazolene and Z is single bond, R₂=R₃=H, then B is not a phenyl, 4-Cl-phenyl, 4-CH₃—O-phenyl or 4-NO₂-phenyl.

4. A compound according to claim 1 having the compound of Formula (Ic): 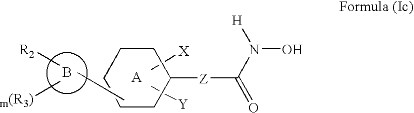
wherein
Z is a single bond or a C₁-C₄ hydrocarbon chain which may contain 0 to 1 double bond or triple bond, unsubstituted or substituted with one or more substituents independently selected from the group consisting of C₁-C₄ alkyl;
A is a six-membered aromatic ring which is selected from the group consisting of optionally substituted arylene or optionally substituted heteroarylene and when Z is a single bond then A is not selected from the group consisting of phenylene and six-membered heteroarylene containing 3 or less than 3 nitrogens;
B is an aromatic ring and is attached to the 3rd or 4^{th} position relative to Z of ring A selected from the group consisting of aryl, and heteroaryl and wherein A and B can not both be phenylene;
wherein A and B are connected via a carbon-carbon bond;
R₂ is selected from the group consisting of halogen, alkyl, alkenyl, alkynyl, haloalkyl, haloalkenyl, heteroalkyl, cycloalkyl, cycloalkenyl, heterocycloalkyl, heterocycloalkenyl, aryl, heteroaryl, cycloalkylalkyl, heterocycloalkylalkyl, arylalkyl, heteroarylalkyl, arylalkenyl, cycloalkylheteroalkyl, heterocycloalkylheteroalkyl, heteroarylheteroalkyl, arylheteroalkyl, hydroxy, hydroxyalkyl, alkoxy, alkoxyalkyl, alkoxyaryl, alkenyloxy, alkynyloxy, cycloalkylkoxy, heterocycloalkyloxy, aryloxy, heteroaryloxy, arylalkyloxy, amino, alkylamino, aminoalkyl, acylamino, arylamino, phenoxy, benzyloxy, COOH, COOR₄, SH, CONHR₄, NHR₄, —(CH₂)ₙNHCOR₄, NHCOR₄, NHCOOR₄ NHCONHR₄, C(═NOH)R₄, NHSOR₄ NHSO₂R₄, —(CH₂)ₙ—NR₆R₇, alkoxycarbonyl, alkylaminocarbonyl, sulfonyl, alkylsulfonyl, alkylsulfinyl, arylsulfonyl, arylsulfinyl, aminosulfonyl, aminosulfinyl, SR₄ and acyl; each of which may optionally be substituted, wherein R₂ does not contain the moiety NHCONHCO or NHCONHSO₂;
R₃ is selected from the group consisting of H, halogen, alkyl, alkenyl, alkynyl, haloalkyl, haloalkenyl, heteroalkyl, cycloalkyl, cycloalkenyl, heterocycloalkyl, heterocycloalkenyl, aryl, heteroaryl, cycloalkylalkyl, heterocycloalkylalkyl, arylalkyl, heteroarylalkyl, arylalkenyl, cycloalkylheteroalkyl, heterocycloalkylheteroalkyl, heteroarylheteroalkyl, arylheteroalkyl, hydroxy, hydroxyalkyl, alkoxy, alkoxyalkyl, alkoxyaryl, alkenyloxy, alkynyloxy, cycloalkylkoxy, heterocycloalkyloxy, aryloxy, heteroaryloxy, arylalkyloxy, amino, alkylamino, aminoalkyl, acylamino, arylamino, phenoxy, benzyloxy, COOH, COOR₄, SH, CONHR₄, NHR₄, —(CH₂)ₙNHCOR₄, NHCOR₄, NHCOOR₄ NHCONHR₄, C(═NOH)R₄, NHSOR₄ NHSO₂R₄, —(CH₂)ₙNR₆R₇, alkoxycarbonyl, alkylaminocarbonyl, sulfonyl, alkylsulfonyl, alkylsulfinyl, arylsulfonyl, arylsulfinyl, aminosulfonyl, aminosulfinyl, SR₄ and acyl; each of which may optionally be substituted wherein R₃ does not contain the moiety NHCONHCO or NHCONHSO₂;
X and Y are the same or different and independently selected from H, halo, C₁-C₄ alkyl, such as CH₃ and CF₃, NO₂, OR₄, SR₄, C(O)R₅, CN, and NR₈ R₉;
R₄ is selected from H, C₁-C₄ alkyl, heteroalkyl, aryl, heteroaryl, acyl;
R₅ is selected from H, C₁-C₄ alkyl;
each R₆ and R₇ is independently selected from the group consisting of H, alkyl, alkenyl, alkynyl, haloalkyl, heteroalkyl, cycloalkyl, heterocycloalkyl, aryl, heteroaryl, cycloalkylalkyl, heterocycloalkylalkyl, arylalkyl, heteroarylalkyl and acyl each of which may be optionally substituted;
R₈ and R₉ are the same or different and independently selected from H, C₁-C₆ alkyl, C₄-C₉ cycloalkyl, C₄-C₉ heterocycloalkyl, aryl, heteroaryl, arylalkyl, heteroarylalkyl;
n is an integer from 0 to 6;
m is an integer from 0 to 4;
or a pharmaceutically acceptable salt or prodrug thereof.

5. A compound according to claim 1 having the Formula (Id): 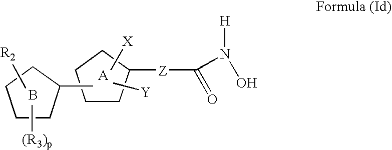
wherein 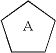
is selected from the group consisting of 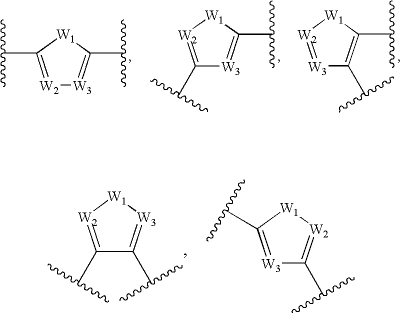
wherein W₁ is selected from the group consisting of O, S and NH;
W₂ and W₃ are independently selected from the group consisting of N, CX and CY;
p is an integer from 0 to 3,
B is a 5-membered heteroarylene,
wherein Z, X, Y, R₂ and R₃ are as described in claim 1, or a pharmaceutically acceptable salt or prodrug thereof.

6. A compound according to claim 1 having the Formula (Ie): 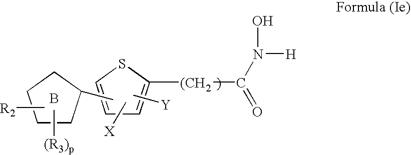
wherein B is a 5-membered heteroarylene, p is an integer from 0 to 3 and X, Y, R₂ and R₃ are the same as in claim 1.

7. A compound according to claim 1 having the Formula (If) 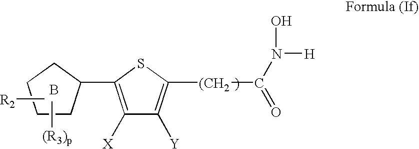
wherein B is a 5-membered heteroarylene, p is an integer from 0 to 3 and X, Y, R₂ and R₃ are the same as in claim 1.

8. A compound according to claim 1 of the Formula (Ig): 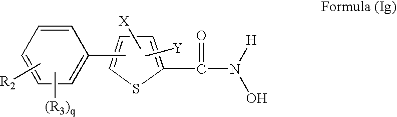
wherein q is an integer from 0 to 4 and X, Y, R₂ and R₃ are the same as in claim 1.

9. A compound according to claim 1 of the Formula (Ih): 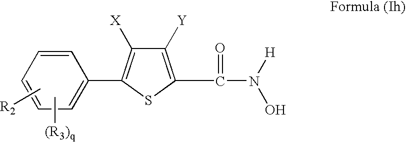
wherein q is an integer from 0 to 4 and X, Y, R₂ and R₃ are the same as in claim 1.

10. A compound according to claim 1 of the Formula (Ii): 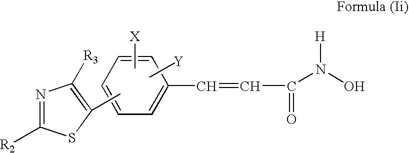
X, Y, R₂ and R₃ are the same as in claim 1.

11. A compound according to claim 1 of the Formula (Ij): 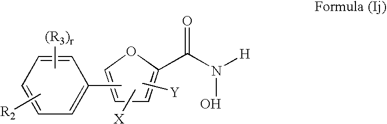
r is an integer from 0 to 4 and X, Y, R₂ and R₃ are the same as in claim 1.

12. A compound according to claim 1 of the Formula (Ik): 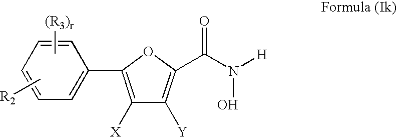
r is an integer from 0 to 4 and X, Y, R₂ and R₃ are the same as in claim 1.

13. A compound according to claim 1 wherein A is an optionally substituted 5-membered heteroarylene ring.

14. A compound according to claim 1 wherein A is an optionally substituted 5-membered heteroarylene ring selected from the group consisting of 2,5-furanylene; 2,4-furanylene; 2,3-furanylene; 3,4-furanylene; 2,5-thiophenylene; 2,4-thiophenylene, 2,3-thiophenylene; 3,4-thiophenylene; 1,2-pyrrolylene; 1,3-pyrrolylene; 1,4-pyrrolylene; 1,5-pyrrolylene; 2,3-pyrrolylene; 2,4pyrrolylene; 2,5-pyrrolylene; 3,4-pyrrolylene; 2,5-oxazolylene; 2,4-oxazolylene; 4,5-oxazolylene, 2,5-thiazolylene; 2,4-thiazolylene; 4,5-thiazolylene 1,2-imidazolylene; 1,4-imidazolylene; 1,5-imidazolylene; 2,4-imidazolylene; 2,5-imidazolylene; 4,5-imidazolylene 1,3-pyrazolylene; 1,4-pyrazolylene; 1,5-pyrazolylene; 3,4-pyrazolylene; 3,5-pyrazolylene; 4,5-pyrazolylene; 3,4-isoxazolylene; 3,5-isoxazolylene; 4,5-isoxazolylene; 3,4-isothiazolylene; 3,5-isothiazolylene; 4,5-isothiazolyIene; 4,5-(1,2,3-oxadiazoly)-ene; 3,5,-(1,2,4-oxadiazolyl)ene; 1,4-(1,2,3-triazolyl)ene; 1,5-(1,2,3-triazolyl)ene; 4,5-(1,2,3-triazolyl)ene; 1,3-(1,2,4-triazolyl)ene; 1,5-(1,2,4-triazolyl)ene; 3,5-(1,2,4-triazolyl)ene; 3,5-(1,2,4-thiadiazolyl)ene; 2,5-(1,3,4-thiadiazolyl)ene, and 1,5-tetrazolylene.

15. A compound according to claim 1 wherein A is an optionally substituted 5-membered heteroarylene selected from the group consisting of 2,5-thiophenylene; 3,5-isoxazolylene; 3,5-pyrazolylene; 2,5-oxazolylene; 3,5-pyrazolylene; 2,5-furanylene and 2,4-thiophenylene.

16. A compound according to claim 1 wherein B is attached to the 3^{rd} or 4^{th} position relative to Z of Ring A.

17. A compound according to claim 1 wherein A is an optionally substituted phenylene or an optionally substituted 6-membered heteroarylene.

18. A compound according to claim 1 wherein B is an optionally substituted 5-membered heteroarylene.

19. A compound according to claim 1 wherein B is an optionally substituted 5-membered heteroarylene ring selected from the group consisting of 2,5-furanylene; 2,4-furanylene; 2,3-furanylene; 3,4-furanylene; 2,5-thiophenylene; 2,4-thiophenylene, 2,3-thiophenylene; 3,4Ahiophenylene; 1,2-pyrrolylene; 1,3-pyrrolylene; 1,4-pyrrolylene; 1,5-pyrrolylene; 2,3-pyrrolylene; 2,4pyrrolylene; 2,5-pyrrolylene; 3,4-pyrrolylene; 2,5-oxazolylene; 2,4-oxazolylene; 4,5-oxazolylene, 2,5-thiazolylene; 2,4-thiazolylene; 4,5-thiazolylene 1,2-imidazolylene; 1,4-imidazolylene; 1,5-imidazolylene; 2,4-imidazolylene; 2,5- imidazolylene; 4,5-imidazolylene 1,3-pyrazolylene; 1,4-pyrazolylene; 1,5-pyrazolylene; 3,4-pyrazolylene; 3,5-pyrazolylene; 4,5-pyrazolylene; 3,4-isoxazolylene; 3,5-isoxazolylene; 4,5-isoxazolylene; 3,4-isothiazolylene; 3,5-isothiazolylene; 4,5-isothiazolylene; 4,5-(1,2,3-oxadiazoly)-ene; 3,5,-(1,2,4-oxadiazolyl)ene; 1,4-(1,2,3-triazolyl)ene; 1,5-(1,2,3-triazolyl)ene; 4,5-(1,2,3-triazolyl)ene; 1,3-(1,2,4-triazolyl)ene; 1,5-(1,2,4-triazolyl)ene; 3,5-(1,2,4-triazolyl)ene; 3,5-(1,2,4-thiadiazolyl)ene; 2,5-(1,3,4-thiadiazolyl)ene, and 1,5-tetrazolylene.

20. A compound according to claim 1 wherein B is an optionally substituted 5-membered heteroarylene selected from the group consisting of 2,4-thiazolylene; 4,2-thiazolylene; 1,3-phenylene; 2,5-thiophenylene and 1,4-phenylene.

21. A compound according to claim 1 Z is a single bond.

22. A compound according to claim 1 Z is CH₂.

23. A compound according to claim 1 Z is CH═CH.

24. A compound according to claim 1 wherein X and Y are both H.

25. A compound according to claim 1 wherein R₂ is independently iselected from the group consisting of: —NH₂, —(CH₂)ₙNHCOR₄, —NHSO₂R₄, —NR₄, —(CH₂)ₙNR₆R₇—, arylalkyl and heteroarylalkyl, each of which may be optionally substituted wherein n is an integer from 0 to 6, and R₄, R₆ and R₇ are as described in claim 1.

26. A compound according to claim 1 wherein R₂ is selected from the group consisting of R₆R₇N—(CH₂)ₙ— wherein n is an integer from 1 to 3.

27. A compound according to claim 26 wherein R₆ and R₇ are independently selected from the group consisting of:
H, cyclopropyl, 2-(4-Hydroxy-3,5-dimethoxy-phenyl)-ethyl, 3-Pyrrolidin-1-yl-propyl, 2-Morpholin-4-yl-ethyl, 3-Morpholin4-yl-propyl, 2-Dimethylamino-ethyl. 4-[4-(2,3-Dimethyl-phenyl)-piperazin-1-ylmethyl, 3-Imidazol-1-yl-propyl, 3-phenyl-propyl, (2-Hydroxy-ethyl)-phenethyl, 2-Hydroxy-ethyl-2-(1H-indol-3-yl)-ethyl, (2-Morpholin-4-yl-ethyl)-phenethyl, 2-(2-methyl-1H-indol-3-yl)-ethyl, 2-(1H-indol-3-yl)-ethyl, pyridin-3-ylmethyl, 3-hydroxy-propyl, 2-pyridin-2-yl-ethyl, 2-pyridin-3-yl-ethyl, pyridin-3-ylmethyl, 2-pyridin-4-yl-ethyl, benzyl, 3-phenyl-propyl, 2-phenoxy-ethyl, morpholin-4-yl, pyridin-2-yl, phenethyl, 2-(4-bromo-phenyl)-ethyl, 2-(4-fluoro-phenyl)-ethyl, 3-imidazol-1-yl-propyl, 2-(1H-imidazol-4-yl)-ethyl, 1H-Benzoimidazol-2-ylmethyl, 2-piperidin-1-yl-ethyl, 2-pyrrolidin-1-yl-ethyl, 2-cyclohex-1-enyl-ethyl, 2-ethyl-hexyl, 2-thiophen-2-yl-ethyl, 3,3-diphenyl-propyl, 2-biphenyl-4-yl-ethyl, -(4-phenoxy-phenyl, 2-(3-phenoxy-phenyl)-ethyl, 2-(2,3-dimethoxy-phenyl, 2-(2,4-dichloro-phenyl)-ethyl, cyclohexylmethyl, hexyl, isobutyl, 3-isopropoxy-propyl, 2-phenoxy-ethyl, 2-isopropoxy-ethyl, 3-methoxy-benzyl, 4-[1,2,3]thiadiazol4-yl-benzyl, 2,4-dichloro-benzyl, 2-(2-methoxy-phenyl)-ethyl, 2-(3-fluoro-phenyl)-ethyl, 2-(2-fluoro-phenyl)-ethyl, 2,2-diphenyl-ethyl, 2-(4-methoxy-phenyl)-ethyl, 2-(3-chloro-phenyl)-ethyl, 4-phenyl-butyl, 3-phenyl-propyl, 3,3-diphenyl-propyl, 3-(4-methyl-piperazin-1-yl, 3-morpholin-4-yl-propyl, 3-(2-oxo-pyrrolidin-1-yl)-propyl, 3-pyrrolidin-1-yl-propyl, tetrahydro-furan-2-ylmethyl, 2-diethylamino-ethyl, 2-dimethylamino-ethyl.

28. A compound according to claim 1 wherein the compound is selected from the group consisting of 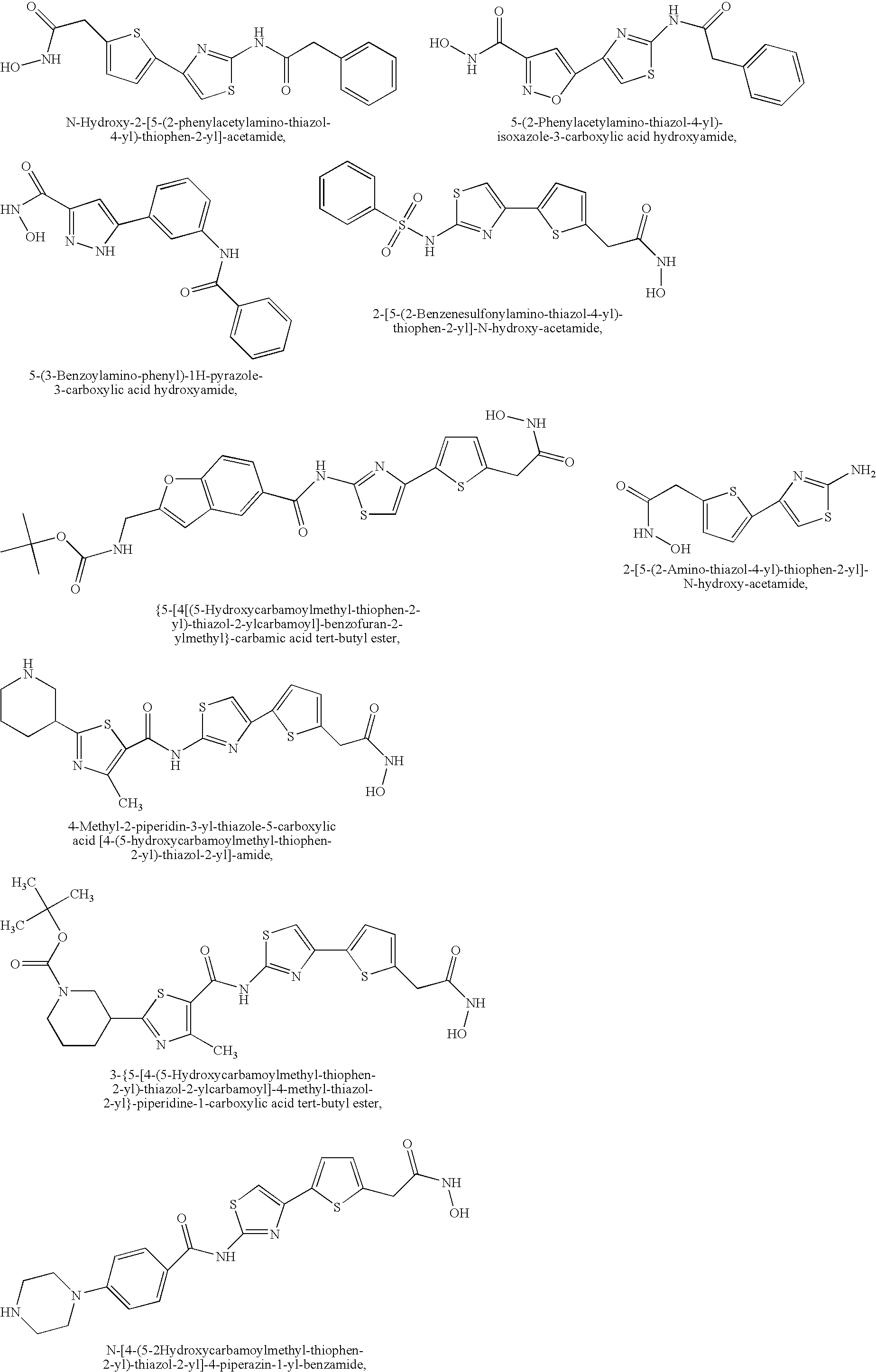 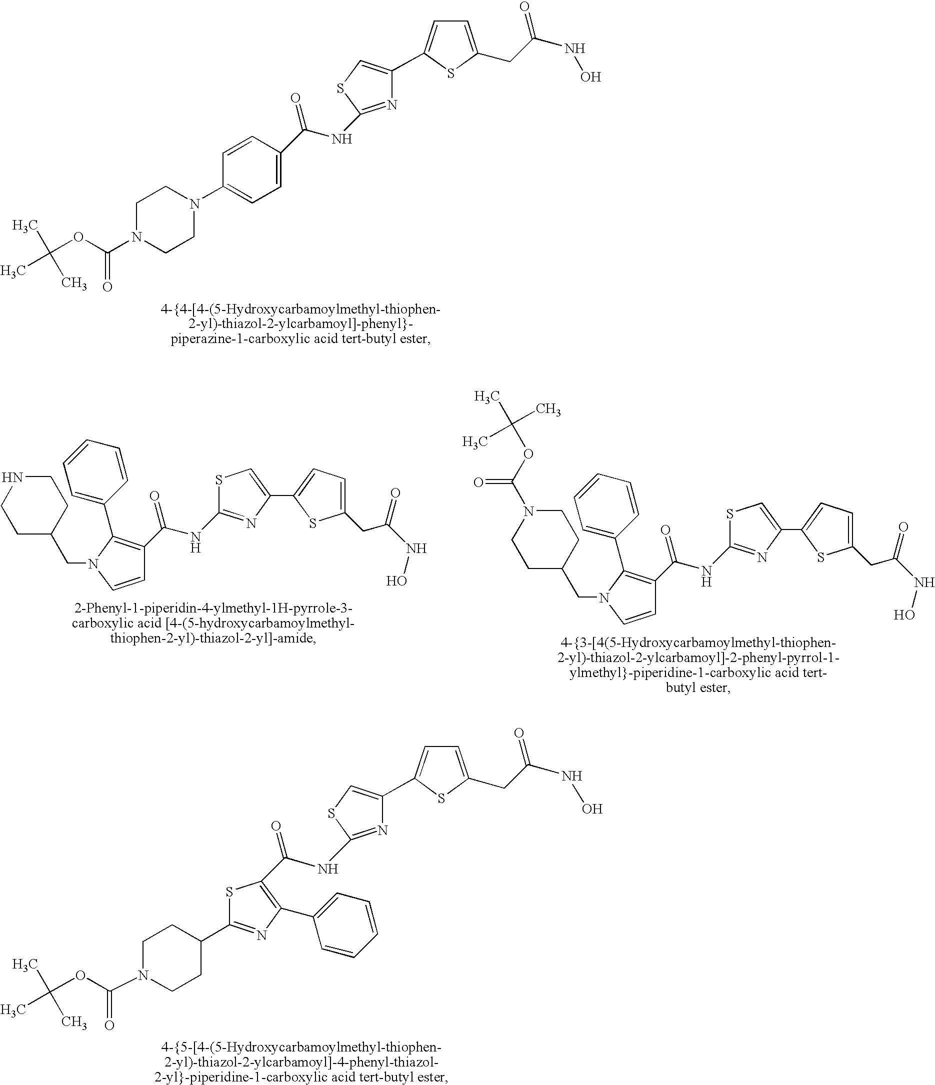 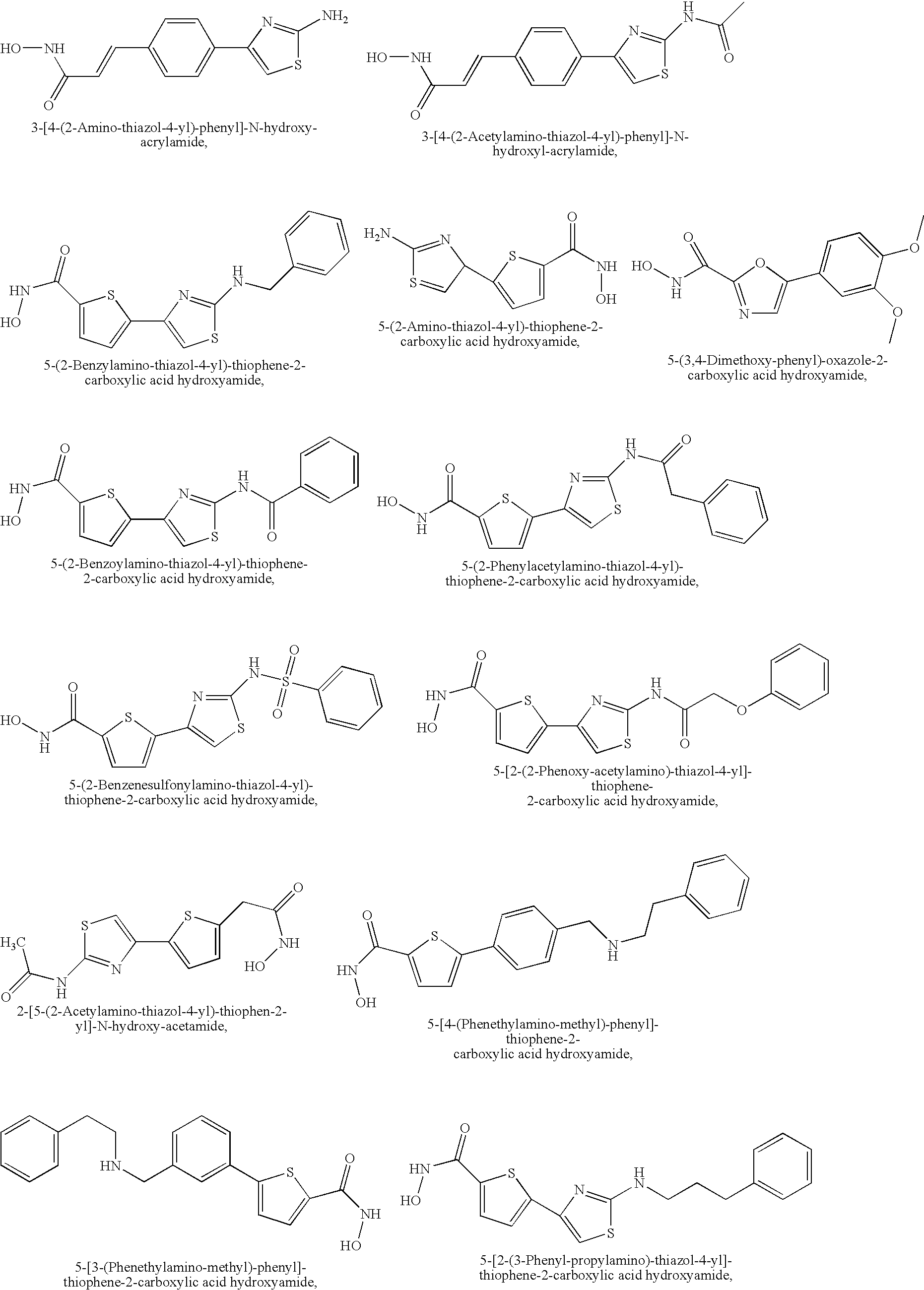 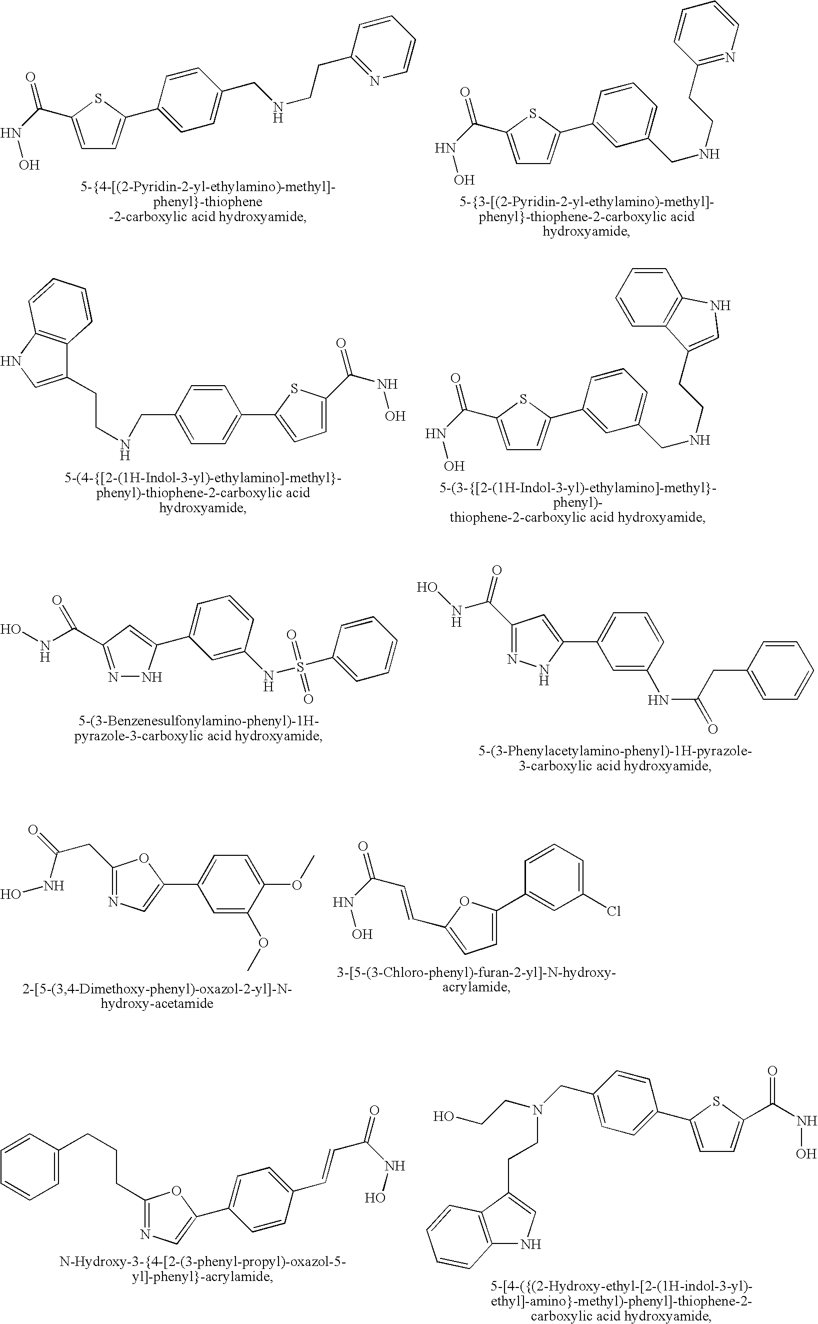 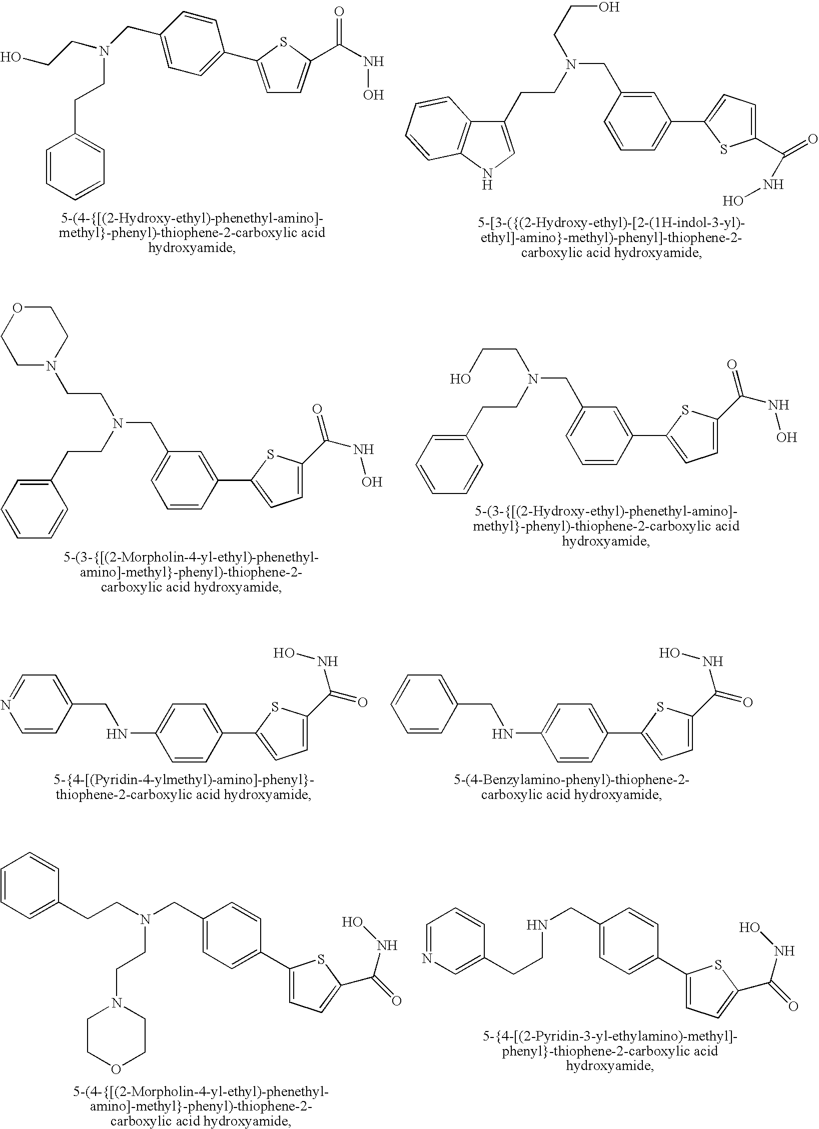 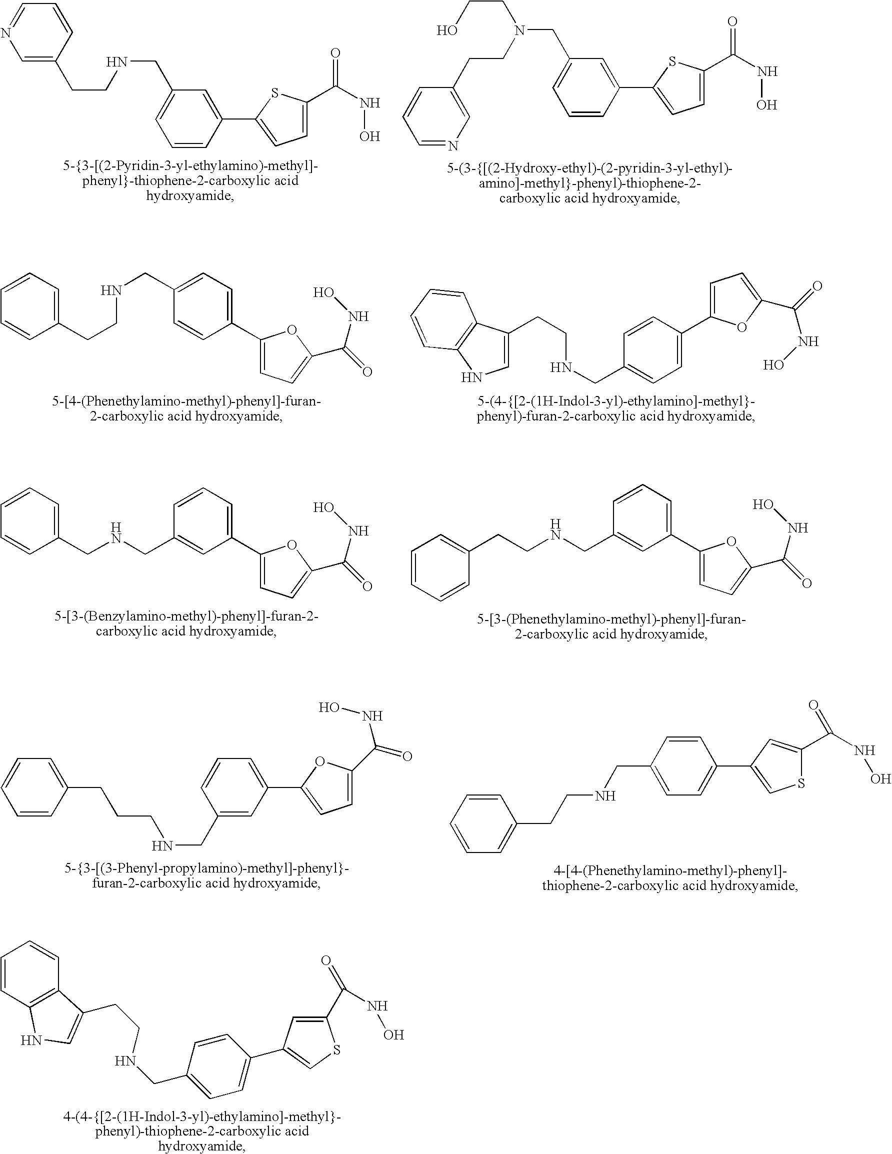
or a pharmaceutically acceptable salt or prodrug thereof.

29. A compound according to claim 1 wherein the compound is selected from the group consisting of 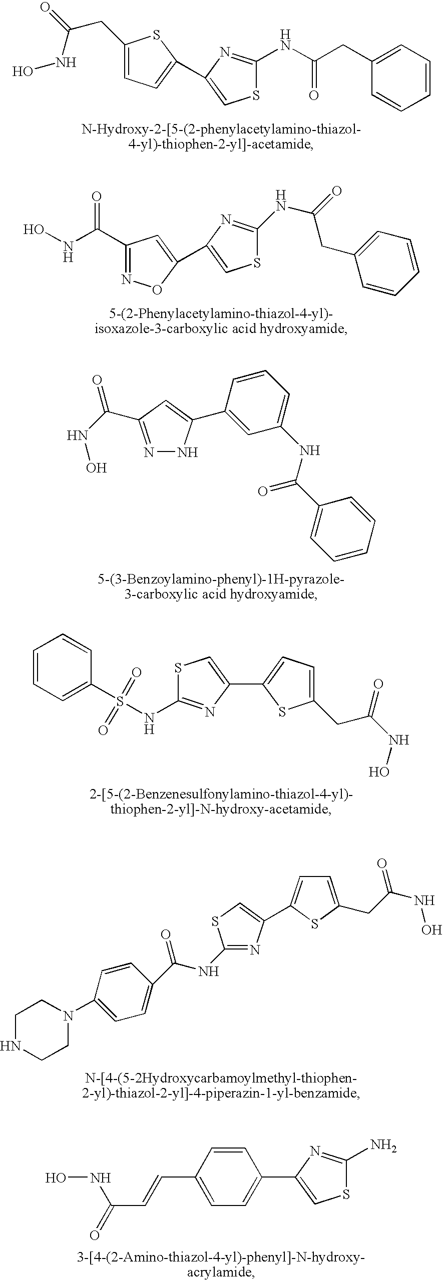 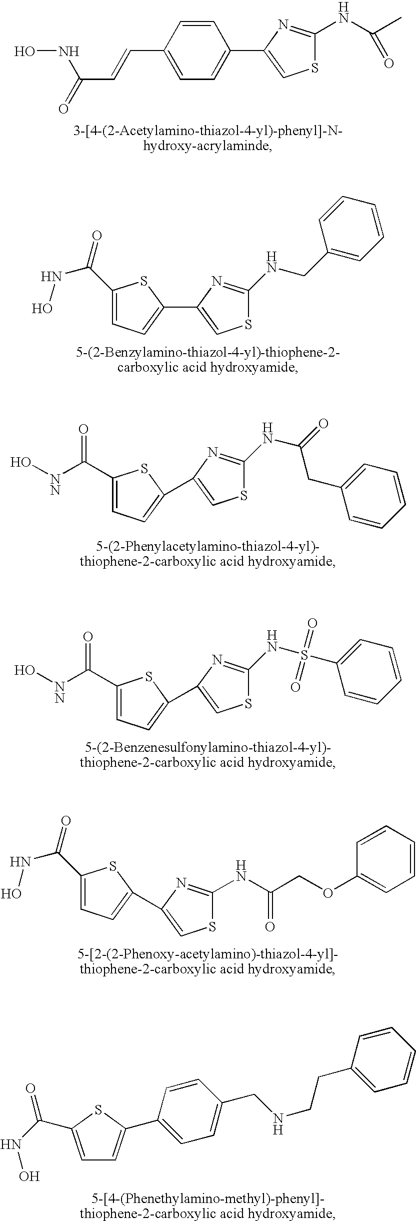 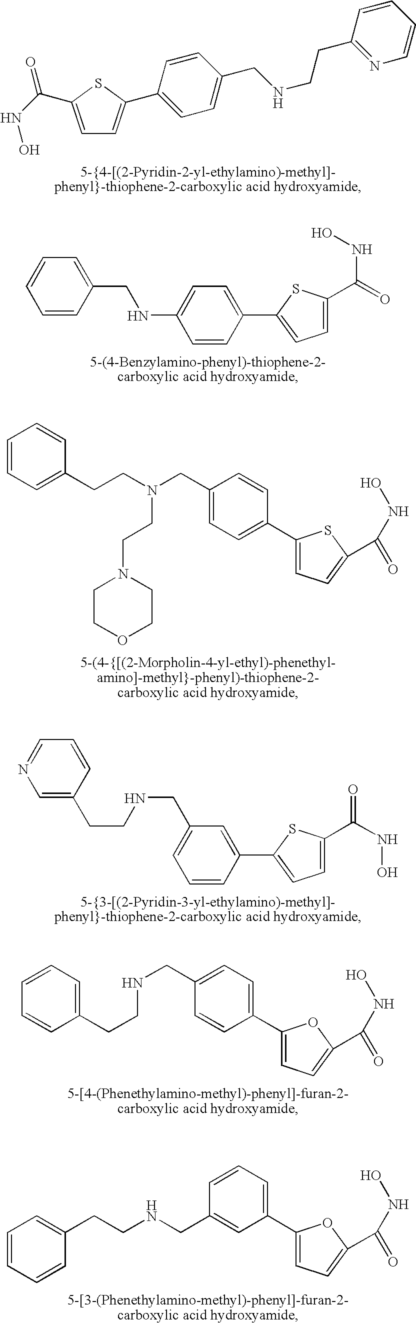 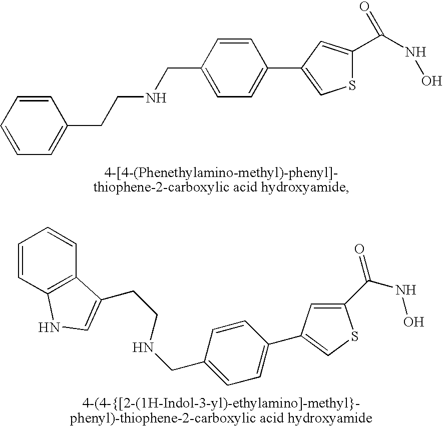
or a pharmaceutically acceptable salt or prodrug thereof.

30. A compound according to claim 1 wherein when Z is a single bond then A is not 2,5-thiophenylene.

31. A compound according to claim 1 wherein when A is phenylene then B is not a 5-membered heteroaryl or 5-membered heteroarylene.

32. A compound according to claim 1 wherein B is not a bicyclic heteroaryl or bicyclic heteroarylene having 9 ring atoms or a hetrocycloalkyl substituted heteroarylene.

33. A compound according to claim 1 wherein when A is a benzimidazole ring, B is not connected to the position 2 of benzimidazole ring.

34. A compound according to claim 1 wherein the optional substituents are independently selected from the group consisting of H, halogen, ═O, ═S, —CN, —NO₂, —CF₃, —OCF₃, alkyl, alkenyl, alkynyl, haloalkyl, haloalkenyl, haloalkynyl, heteroalkyl, cycloalkyl, cycloalkenyl, heterocycloalkyl, heterocycloalkenyl, aryl, heteroaryl, hydroxy, hydroxyalkyl, alkoxy, alkoxyalkyl, alkoxyaryl, alkoxyheteroaryl, alkenyloxy, alkynyloxy, cycloalkyloxy, cycloalkenyloxy, heterocycloalkyloxy, heterocycloalkenyloxy, aryloxy, heteroaryloxy, arylalkyl, heteroarylalkyl, cycloalkylalkyl, heterocycloalkylalkyl, arylalkyloxy, amino, alkylamino, acylamino, aminoalkyl, arylamino, sulfonylamino, sulfinylamino, sulfonyl, alkylsulfonyl, arylsulfonyl, aminosulfonyl, aminoalkyl, alkoxyalky, CH₂heterocycloalkylCOOR₁₀heterocycloalkylCOOR₁₀, —COOH, —COR₅, —C(O)OR₅, CONHR₅, —C(O)C(O)OR₅, C(O)CONHR₅, CON(R₅)OR₅, COCON(R₅)OR₅, NHCOR₅, CH₂NCOOR₁₀, NHCOOR₅, NHCONHR₅, C(═NOH)R₅, —SH, —SR₅, —OR₅ and acyl;
each R₅ is independently selected from the group consisting of H, alkyl, alkenyl, alkynyl, haloalkyl, heteroalkyl, cycloalkyl, heterocycloalkyl, aryl, heteroaryl, cycloalkylalkyl, heterocycloalkylalkyl, arylalkyl, heteroarylalkyl and acyl each of which may be optionally substituted;
R₁₀ is selected from H, alkyl, acyl and aryl.

35. A pharmaceutical composition including a compound according to claim 1 and a pharmaceutically acceptable diluent, excipient or carrier.

36-39. (canceled)

40. A method of treatment of a disorder caused by, associated with or accompanied by disruptions of cell proliferation and/or angiogenesis in a patient the method including administration of a therapeutically effective amount of a compound according to claim 1 to the patient.

41. A method according to claim 40 wherein the disorder is a proliferative disorder.

42. A method according to claim 40 wherein the disorder is cancer.

43. A method according to claim 42 wherein the cancer is selected from breast cancer, lung cancer, ovarian cancer, prostate cancer, head and neck cancer, renal cancer, gastric cancer, colon cancer, pancreatic cancer and brain cancer.

44-48. (canceled)

49. A method of modifying deacetylase activity including contacting the deacetylase with a compound according to claim 1.

50. A method according to claim 49 wherein the deacetylase activity is histone deacetylase activity.

51. A method according to claim 49 wherein the deacetylase activity is class I histone deacetylase activity.

52. A method according to claim 50 wherein the histone deacetylase is HDAC1.

53. A method according to claim 50 wherein the histone deacetylase is HDAC8.

54. A method of treatment of a disorder that can be treated by the inhibition of deacetylase activity in a patient including administration of a therapeutically effective amount of a compound according to claim 1 to the patient.

55. A method according to claim 54 wherein the deacetylase activity is histone deacetylase activity.

56. A method of treatment of a disorder that is mediated by histone deacetylase activity in a patient including administration of a therapeutically effective amount of a compound according to claim 1 to the patient.

57. A method according to claim 54 wherein the disorder is selected from the group consisting of proliferative disorders (e.g. cancer); Neurodegenerative diseases including Huntington's Disease, Polyglutamine diseases, Parkinson's Disease, Alzheimer's Disease, Seizures, Striatonigral degeneration, Progressive supranuclear palsy, Torsion dystonia, Spasmodic torticollis and dyskinesis, Familial tremor, Gilles de la Tourette syndrome, Diffuse Lewy body disease, Progressive supranuclear palsy, Pick's disease, Intracerebral haemorrhage, Primary lateral sclerosis, Spinal muscular atrophy, Amyotrophic lateral sclerosis, Hypertrophic interstitial polyneuropathy, Retinitis pigmentosa, Hereditary optic atrophy, Hereditary spastic paraplegia, Progressive ataxia and Shy-Drager syndrome; Metabolic diseases including Type 2 diabetes; Degenerative Diseases of the Eye including Glaucoma, Age-related macular degeneration, Rubeotic glaucoma, Intersitital keratitis, diabetic retinopathy; Inflammatory diseases and/or Immune system disorders including Rheumatoid Arthritis (RA), Osteoarthritis, Juvenile chronic arthritis, Graft versus Host disease, Psoriasis, Asthma, Spondyloarthropathy, Crohn's Disease, Inflammatory bowel disease, Colitis Ulcerosa, Alcoholic hepatitis, Diabetes, Sjoegrens's syndrome, Multiple Sclerosis, Ankylosing Membranous glomerulopathy, Discogenic pain, Systemic Lupus Erythematosus; Disease involving angiogenesis including cancer, psoriasis, rheumatoid arthritis; Psychological disorders including bipolar disease, schizophrenia, mania, depression and dementia; Cardiovascular Diseases including Heart failure, restenosis and arteriosclerosis; Fibrotic diseases including liver fibrosis, cystic fibrosis and angiofibroma; Infectious diseases including Fungal infections, such as Candida Albicans, Bacterial infections, Viral infections, such as Herpes Simplex, Protozoal infections, such as Malaria, Leishmania infection, Trypanosoma brucei infection, Toxoplasmosis and coccidiosis and Haematopoietic disorders including thalassemia, anemia and sickle cell anemia.

58. A method for inhibiting cell proliferation including administration of an effective amount of a compound according to claim 1.

59. A method of treatment of a neurodegenerative disorder in a patient including administration of a therapeutically effective amount of a compound according to claim 1 to the patient.

60. A method according to claim 59 wherein the neurodegenerative disorder is Huntington's Disease.

61. A method of treatment of an inflammatory disease and/or immune system disorder in a patient including administration of a therapeutically effective amount of a compound according to claim 1 to the patient.

62. A method according to claim 61 wherein the inflammatory disease and/or immune system disorder is rheumatoid arthritis.

63. A method according to claim 61 wherein the inflammatory disease and/or immune system disorder is systemic lupus erythematosus.

64. A method of treatment of a proliferative disorder in patient including administration of a therapeutically effective amount of a compound according to claim 1 to the patient.

65. A method of treatment of cancer in patient including administration of a therapeutically effective amount of a compound according to claim 1 to the patient.

66. A method according to claim 65 wherein the cancer is a hematologic malignancy.

67. A method according to claim 66 wherein the hematologic malignancy is selected from a group consisting of B-cell lymphoma, T-cell lymphoma and leukemia.

68. A method according to claim 65 wherein the cancer is a solid tumor.

69. A method according to claim 67 wherein the solid tumor is selected from a group consisting of breast cancer, lung cancer, ovarian cancer, prostate cancer, head and neck cancer, renal cancer, gastric cancer, colon cancer, pancreatic cancer and brain cancer.

70-75. (canceled)

76. A method for the induction of apoptosis of tumor cells including contacting the tumor cells with an effective amount of a compund according to claims 1.

77. A method according to claim 51 wherein the histone deacetylase is HDAC1.

78. A method according to claim 51 wherein the histone deacetylase is HDAC8.

79. A method according to claim 55 wherein the disorder is selected from the group consisting of proliferative disorders (e.g. cancer); Neurodegenerative diseases including Huntington's Disease, Polyglutamine diseases, Parkinson's Disease, Alzheimer's Disease, Seizures, Striatonigral degeneration, Progressive supranuclear palsy, Torsion dystonia, Spasmodic torticollis and dyskinesis, Familial tremor, Gilles de la Tourette syndrome, Diffuse Lewy body disease, Progressive supranuclear palsy, Pick's disease, Intracerebral haemorrhage, Primary lateral sclerosis, Spinal muscular atrophy, Amyotrophic lateral sclerosis, Hypertrophic interstitial polyneuropathy, Retinitis pigmentosa, Hereditary optic atrophy, Hereditary spastic paraplegia, Progressive ataxia and Shy-Drager syndrome; Metabolic diseases including Type 2 diabetes; Degenerative Diseases of the Eye including Glaucoma, Age-related macular degeneration, Rubeotic glaucoma, Intersitital keratitis, diabetic retinopathy; Inflammatory diseases and/or Immune system disorders including Rheumatoid Arthritis (RA), Osteoarthritis, Juvenile chronic arthritis, Graft versus Host disease, Psoriasis, Asthma, Spondyloarthropathy, Crohn's Disease, Inflammatory bowel disease, Colitis Ulcerosa, Alcoholic hepatitis, Diabetes, Sjoegrens's syndrome, Multiple Sclerosis, Ankylosing spondylitis, Membranous glomerulopathy, Discogenic pain, Systemic Lupus Erythematosus; Disease involving angiogenesis including cancer, psoriasis, rheumatoid arthritis; Psychological disorders including bipolar disease, schizophrenia, mania, depression and dementia; Cardiovascular Diseases including Heart failure, restenosis and arteriosclerosis; Fibrotic diseases including liver fibrosis, cystic fibrosis and angiofibroma; Infectious diseases including Fungal infections, such as Candida Albicans, Bacterial infections, Viral infections, such as Herpes Simplex, Protozoal infections, such as Malaria, Leishmania infection, Trypanosoma brucei infection, Toxoplasmosis and coccidiosis and Haematopoietic disorders including thalassemia, anemia and sickle cell anemia.

80. A method according to claim 56 wherein the disorder is selected from the group consisting of proliferative disorders (e.g. cancer); Neurodegenerative diseases including Huntington's Disease, Polyglutamine diseases, Parkinson's Disease, Alzheimer's Disease, Seizures, Striatonigral degeneration, Progressive supranuclear palsy, Torsion dystonia, Spasmodic torticollis and dyskinesis, Familial tremor, Gilles de la Tourette syndrome, Diffuse Lewy body disease, Progressive supranuclear palsy, Pick's disease, Intracerebral haemorrhage, Primary lateral sclerosis, Spinal muscular atrophy, Amyotrophic lateral sclerosis, Hypertrophic interstitial polyneuropathy, Retinitis pigmentosa, Hereditary optic atrophy, Hereditary spastic paraplegia, Progressive ataxia and Shy-Drager syndrome; Metabolic diseases including Type 2 diabetes; Degenerative Diseases of the Eye including Glaucoma, Age-related macular degeneration, Rubeotic glaucoma, Intersitital keratitis, diabetic retinopathy; Inflammatory diseases and/or Immune system disorders including Rheumatoid Arthritis (RA), Osteoarthritis, Juvenile chronic arthritis, Graft versus Host disease, Psoriasis, Asthma, Spondyloarthropathy, Crohn's Disease, Inflammatory bowel disease, Colitis Ulcerosa, Alcoholic hepatitis, Diabetes, Sjoegrens's syndrome, Multiple Sclerosis, Ankylosing spondylitis, Membranous glomerulopathy, Discogenic pain, Systemic Lupus Erythematosus; Disease involving angiogenesis including cancer, psoriasis, rheumatoid arthritis; Psychological disorders including bipolar disease, schizophrenia, mania, depression and dementia; Cardiovascular Diseases including Heart failure, restenosis and arteriosclerosis; Fibrotic diseases including liver fibrosis, cystic fibrosis and angiofibroma; Infectious diseases including Fungal infections, such as Candida Albicans, Bacterial infections, Viral infections, such as Herpes Simplex, Protozoal infections, such as Malaria, Leishmania infection, Trypanosoma brucei infection, Toxoplasmosis and coccidiosis and Haematopoietic disorders including thalassemia, anemia and sickle cell anemia.
